(19) United States
(12) Patent Application Publication
(10) Pub. No.: US 2010/0049180 A1
(43) Pub. Date: Feb. 25, 2010

(54) SYSTEM AND METHOD FOR CONDITIONING ANIMAL TISSUE USING LASER LIGHT

(75) Inventors: Jonathon D. Wells, Seattle, WA (US); E. Duco Jansen, Nashville, TN (US); Gerald J. Wilmink, San Antonio, TN (US); Jeffrey M. Davidson, Nashville, TN (US); Charles A. Lemaire, Apple Valley, MN (US)

Correspondence Address: LEMAIRE PATENT LAW FIRM, P.L.L.C., P.O. BOX 1818, BURNSVILLE, MN, 55337, US

(73) Assignees: LOCKHEED MARTIN CORPORATION, Bethesda, MD (US); VANDERBILT UNIVERSITY, Nashville, TN (US)

(21) Appl. No.: 12/254,832

(22) Filed: Oct. 20, 2008

Related U.S. Application Data
(60) Provisional application No. 60/981,405, filed on Oct. 19, 2007.

(51) Int. Cl. A61B 18/20

(52) U.S. Cl. 606/12; 606/11

(57) ABSTRACT

Systems and methods for prophylactic measures aimed at improving wound repair. In some embodiments, laser-mediated preconditioning would enhance surgical wound healing that was correlated with hsp70 expression. Using a pulsed laser (λ=1850 nm, Tp=2 ms, 50 Hz, H=7.64 mJ/cm²) the skin of transgenic mice that contain an hsp70 promoter-driven luciferase were preconditioned 12 hours before surgical incisions were made. Laser protocols were optimized using temperature, blood flow, and hsp70-mediated bioluminescence measurements as benchmarks. Bioluminescent imaging studies in vivo indicated that an optimized laser protocol increased hsp70 expression by 15-fold. Under these conditions, healed areas from incisions that were laser-preconditioned were two times stronger than those from control wounds. Our data suggest that these methods can provide effective and improved tissue-preconditioning protocols and that mild laser-induced heat shock that correlated with an expression of Hsp70 may be a useful therapeutic intervention prior to or after surgery.

### RELATED APPLICATIONS

This application claims benefit to U.S. Provisional Patent Application 60/981,405 titled “LASER PRECONDITIONING FOR WOUND REPAIR ENHANCEMENT”, filed Oct. 19, 2007 by Gerald J. Wilmink, Jeffrey M. Davidson, E. Duco Jansen, and Jonathon D. Wells, which is incorporated by reference in its entirety.

### STATEMENT OF GOVERNMENT INTEREST

This invention was made with government support under grants 5P30 AR041043 awarded by the NIH and F49620-01-1-0429 and FA9550-04-1-0045 awarded by the DOD MFEL Program. The government has certain rights in the invention.

### FIELD OF THE INVENTION

The present invention relates to systems and methods for laser medical treatments and more specifically to in vivo pre-trauma and post trauma conditioning of animal tissue (such as human tissue) using laser light for modulation and enhancement of healing.

### BACKGROUND OF THE INVENTION

The broad gain bandwidth of conventional fiber-laser systems allows for operation over a wide range of wavelengths, or even tunable operation. For the simplest fiber-laser system with cavity mirrors having reflectivity across a broad range of wavelengths, the output wavelength can be very broad and can vary with pump power, fiber length, and/or other parameters. The power that can be generated from fiber lasers and fiber-laser amplifiers can often be limited by nonlinear optical effects in the gain and/or delivery fibers used in the system.

It is desirable to produce high peak and average powers from fiber lasers and amplifiers. Stimulated Brillouin scattering (SBS) and other nonlinear effects such as self-phase modulation (SPM), four-wave mixing (FWM), and stimulated Raman scattering (SRS) are the main effects limiting the output power and pulse energy of a fiber amplifier or laser. To suppress these effects in a fiber amplifier/laser, it is desirable to use a rare-earth-doped (RE-doped) double-clad fiber with a large core. The large core provides two benefits: Spreading the light over a larger core decreases the intensity driving the nonlinear processes, and increasing the core/cladding diameter ratio increases pump absorption, enabling the shortening of the fiber to further reduce nonlinearities. When good beam quality is required, however, increasing the core diameter of the fiber requires that the fiber numerical aperture (NA) be decreased, in order that higher-order modes cannot propagate in the fiber. Using relatively large-core, low-NA fibers with mode-filtering techniques has been demonstrated to achieve good beam quality, but there are practical disadvantages to the use of such fibers. Fibers with very low values of NA exhibit large bending losses, even for relatively large-radius bends. With fibers having the lowest NA, the fiber must be kept quite straight, otherwise the optical amplifier and/or laser has very low efficiency as the bending loss becomes too high. Since a typical laser oscillator or amplifier might require on the order of a meter or more of gain fiber, the inability to coil the fiber has precluded compact packaging of the fiber-laser system.

Diode lasers are available in a wide range of output wavelengths, from GaN lasers operating in the near UV (e.g., 395- to 405-nm wavelengths) to those using GaAs and related materials operating in the mid to long wave infrared (2000 nm or longer wavelengths). Wavelength multipliers are available to generate shorter wavelengths, for example as described in U.S. patent application Ser. No. 11/558,362 and U.S. patent application Ser. No. 11/558,362, which are all incorporated herein by reference.

Stimulated Brillouin Scattering (SBS) is a well-known phenomenon that can lead to power limitations or even the destruction of a high-power fiber-laser system due to sporadic or unstable feedback, self-lasing, pulse compression and/or signal amplification.

Wound repair enhancement can apparently be achieved using heat applied to the general area of the wound, wherein the heat produces a slight tissue “insult” (trauma or injury) that triggers the body to generate a healing response that may enhance the strength of the wound repair, that may enhance the cosmetic result by reducing certain characteristics (e.g., size, hardness and/or discolorization) of scar tissue, and/or may reduce the time needed for healing.

Several types of sub-lethal stimuli such as hyperthermia (Morimoto et al., 1996), desiccation, ATP depletion (Kabakov et al., 2002), and ischemia (Wirth et al., 1996) can induce a sub-lethal stress response. The cellular response mechanism to thermal stress includes production of heat-shock proteins (HSPs). The expression of these molecular chaperones is activated immediately after exposure to elevated temperatures and reaches a maximum several hours later. HSPs are found in all organisms and comprise a large family of cytoplasmic proteins ranging from 20 to 120 kilodaltons. These molecular chaperones play an integral role in maintaining intracellular homeostasis by assisting in protein folding and by mediating processes which protect the cell from further injury (Morimoto et al., 1996).

As used herein, “HSP” refers to a heat-shock protein. As used herein, the terms “Hsp 70” and “Hsp70 protein” (using initial capital letters) refer to a specific heat-shock protein, and “hsp70” (lowercase italics) refers to the gene (a noun) that causes production of Hsp70 protein, the hsp70 gene's expression (a verb, also called “hsp70 expression”), the hsp70 gene's promoter (a noun, also called the “hsp70 promoter”), and the hsp70 gene's upregulation (a verb, also called the “hsp70 upregulation”), wherein the meanings of each will be apparent to one of skill in the art from the context in which they are used. The term “promoter” refers to a part of a part of the gene-a gene promoter is the sequence of nucleotides that initiates transcription of a gene (think of this as the “ignition key”). Something else (another factor or stimulus) interacts with the promoter region of a gene and “turns it on”—this results in the production of mRNA which is subsequently translated to protein. Oftentimes further processing of the protein takes place (“posttranslational modification”) to arrive at the final (functional) protein(s). The term “upregulation” (also called “up-regulation”) refers to enhanced transcription of the gene (into mRNA), which is driven by the gene promoter. Normally (but not necessarily) this results in more protein. The term “expression” refers to the operation that turns the gene “on” and usually results in the production of protein at some level. Upregulation of the gene expression thus refers to enhanced transcription of the gene into mRNA, which normally (but not necessarily) results in more protein as compared to some baseline production of the protein. The term “murine model” refers to an animal (such as a mouse) or a tissue culture (such as mouse dermal fibroblast cells) belonging to the Muridae, the family of rodents that includes the mice and rats, wherein the animal exhibits characteristics (such as the production of a luciferase light-producing enzyme triggered by the activation of a luc transgene in the tissue) that demonstrate or characterize some process that can be used to model a similar process in another animal (such as a human). The term “fold induction” refers to the amount of increase, for example a 2-fold induction produces twice as much protein as a baseline production, while an 18-fold induction produces eighteen times as much protein as the baseline production. A normalization procedure is used to determine a “fold induction number” (a quotient) which is indicative of the relative magnitude of hsp70 expression resulting from various tissue-treatment protocols compared to normal, untreated tissue.

The hsp70 gene and Hsp70 protein are the most highly induced targets of heat shock and the best characterized HSP (Beckham et al., 2004; Pockley, 2002). Due to its marked induction, hsp70 expression is commonly used as a sensitive indicator of thermal damage to cells (Beckham et al., 2004; Desmettre et al., 2001). The kinetics of hsp70 upregulation are directly related to the hyperthermic regimen, dependent on both temperature and exposure time, and hsp70 is induced by temperature increases of 5-6° C. (Beckham et al., 2004; Morimoto et al., 1996; O'Connell-Rodwell et al., 2004). Although the kinetics of hsp70 expression vary depending on the organism, tissue, and cell type, some general trends are evident (Wilmink et al., 2006). First, the magnitude of hsp70 expression increases in response to elevated thermal stress until a thermal threshold is reached, followed by a subsequent decrease. Secondly, peak hsp70 expression is biphasic, with maxima occurring between 8-12 hours and then approximately 24 hours after thermal stress (Diller, 2006; Wilmink et al., 2007). Third, severe levels of thermal stress may delay hsp70 expression as cellular machinery used to produce Hsp70 protein is damaged.

There is evidence that pre-treating cells or tissue with an initial mild thermal elevation elicits a stress response that can serve to protect the tissue from subsequent lethal stresses (Kim et al., 2004; Li et al., 2003; Topping et al., 2001) or can indeed improve wound healing (Capon & Mordon, 2003). This process of pretreating tissue is commonly referred to as “preconditioning.” Preconditioned cells exhibit greater survivability than untreated cells when exposed to subsequent stresses (Bowman et al., 1997). The beneficial effect of preconditioning is believed to be due to increased production of HSPs, as first described by Ritossa in 1962 (Ritossa, 1962). Since increased hsp70 expression is induced by stressors such as heat, it is hypothesized that its increased expression conveys increased cellular protection (Topping et al., 2001). After an initial thermal stress, Hsp70 protein stabilizes the cell by tending to the recently denatured proteins and by preventing the production of misfolded proteins (Wynn et al., 1994). Hsp70 protein also functions at key regulatory points in the control of apoptosis, thereby inhibiting cell death and promoting cell survival (Bowman et al., 1997; Jaattela and Wissing, 1992; Mosser et al., 1997; Samali and Cotter, 1996).

Tissue-preconditioning protocols have been effectively incorporated into surgical procedures (Snoeckx et al., 2001), the recovery of thermally injured tissues (Baskaran et al., 2001; Merchant et al., 1998; Seppa et al., 2004), protection to ischemia reperfusion injury (Currie et al., 1988; Gowda et al., 1998; Rylander et al., 2005), and even for cancer therapies (Rylander et al., 2006; Wang et al., 2004).

Conventional heat treatments and tissue-preconditioning protocols tend to be coarse (not well controlled in terms of the area treated, the temperature applied, the rate of temperature change and/or the like).

In some embodiments, the present invention uses devices and methods and provides improvements to those devices and methods such as described in U.S. Pat. No. 5,616,140, issued Apr. 1, 1997, and titled “METHOD AND APPARATUS FOR THERAPEUTIC LASER TREATMENT,” U.S. Pat. No. 5,021,452, issued Jun. 4, 1991, and titled “PROCESS FOR ENHANCING WOUND HEALING,” U.S. Pat. No. 7,051,738, issued May 30, 2006, and titled “APPARATUS FOR PROVIDING ELECTROMAGNETIC BIOSTIMULATION OF TISSUE USING OPTICS AND ECHO IMAGING,” U.S. Pat. No. 5,993,378, issued Nov. 30, 1999, and titled “ELECTRO-OPTICAL INSTRUMENTS AND METHODS FOR TREATING DISEASE,” U.S. Pat. No. 5,775,572, issued Jul. 7, 1998, and titled “CARTON WITH CENTER PARTITION,” U.S. Pat. No. 6,165,205, issued Dec. 26, 2000, and titled “METHOD FOR IMPROVED WOUND HEALING,” U.S. Pat. No. 7,351,252, issued Apr. 1, 2008, and titled “METHOD AND APPARATUS FOR PHOTOTHERMAL TREATMENT OF TISSUE AT DEPTH,” U.S. Pat. No. 7,177,695, issued Feb. 13, 2007, and titled “EARLY STAGE WOUND HEALING USING ELECTROMAGNETIC RADIATION,” International Patent Application Publication WO 2007/080239 A1, published Jul. 19, 2007, and titled “SYSTEM FOR TREATMENT OF SKIN WOUNDS, DRESSING, AND BIOCHEMICAL ACTIVATION EQUIPMENT FOR THE USE OF SUCH A SYSTEM,” U.S. Pat. No. 5,140,984, issued Aug. 25, 1992, and titled “LASER HEALING METHOD AND APPARATUS,” and U.S. Patent Application Publication 2004/0093042 A1, published May 13, 2004 and titled “METHOD AND APPARATUS FOR PHOTOTHERMAL TREATMENT OF TISSUE AT DEPTH,” each of which is incorporated herein by reference.

There is a need for improved laser methods and systems, and treatment protocols, particularly fiber-lasers and optical amplifiers, controllers, feedback mechanisms, imaging and scanning methods that allow a care provider to precisely control the area and depth of tissue being treated, to selectively apply different temperatures and treatment times to different areas of the patient's tissue, to selectively apply tissue-preconditioning protocols to external tissues (such as the epidermis and dermis) and/or to selectively apply tissue-preconditioning protocols to internal tissues and structures with minimal invasiveness (for example, in preparation of cardiac or other pre-planned surgeries), and the like. There is also a need for improved laser methods and systems, and treatment protocols that allow a care provider to precisely control the area and depth of tissue being treated after a wound is sustained or surgery is performed.

### SUMMARY OF THE INVENTION

In some embodiments, the present invention provides systems and methods for prophylactic measures aimed at improving wound repair. In some embodiments, laser-mediated preconditioning enhances surgical wound healing that was correlated with hsp70 expression. Using a pulsed diode laser (λ=1850 nm, Tp=2 ms, 50 Hz, H=7.64 mJ/cm²) the skin of transgenic mice that contain an hsp70-promoter-driven luciferase were preconditioned 12 hours before surgical incisions were made. Laser protocols were optimized in vitro and in vivo using temperature, blood flow, and hsp70-mediated bioluminescence measurements as benchmarks. Biomechanical properties and histological parameters of wound healing were evaluated for up to 14 days. Bioluminescent imaging studies in vivo indicated that an optimized laser protocol increased hsp70 expression by 15-fold. Under these conditions, laser-preconditioned incisions were two times stronger than control wounds. Our data suggest that this novel molecular-imaging approach provides a quantitative method for optimization of tissue preconditioning and that mild laser-induced heat shock that correlated with an expression of Hsp70 protein may be a useful therapeutic intervention prior to surgery.

In some embodiments, the present invention provides an apparatus that includes a laser device configured to provide a therapeutically effective dose of laser radiation to a first area of tissue of an animal, wherein the dose of laser radiation is controlled to improve a healing response to an injury of the animal. In some embodiments, the animal is a human. In some embodiments, the laser radiation is in the infrared wavelengths. In some embodiments, the laser radiation has a wavelength between about 1800 nm and about 2000 nm. In some such embodiments, the laser radiation has a wavelength between about 1830 nm and about 1950 nm. In some such embodiments, the laser radiation has a wavelength between about 1840 nm and about 1940 nm. In some embodiments, the wavelength is selected such that the penetration depth and area of laser irradiation are matched with the target tissue volume that needs to be conditioned. For example, for human skin the thickness of tissue to be conditioned is about one to four millimeters (mm), so in some embodiments, the device is configured to achieve that tissue-penetration depth. In some embodiments, the penetration depth is defined as the depth at which the light intensity is 1/e (about 37%) of the intensity at the tissue surface. Some wavelengths used to obtain a 1-4 mm tissue-penetration depth are: around 1800-1840 nm or around 2200 nm in the infrared when targeting water as the chromophore. Alternatively, near infrared or visible wavelengths such as 980 nm or 700-850 nm, could target highly vascularized tissue having high concentrations of hemoglobin or deoxyhemoglobin in tissue and get the desired effective penetration depth based on the principles of light scattering and absorption. In some embodiments, melanin is a prime absorber that affects penetration depths. Thus, some embodiments measure melanin (e.g., by having the user input a classification of the skin color or having an instrument (such as a camera) measure skin color), wherein this value is used to calibrate the laser dose used to achieve a desired temperature.

Some embodiments of the apparatus further include a scanner mechanism configured to scan a laser beam from the laser device relative to the laser device in a scan pattern across an area of tissue larger than the laser beam. In some embodiments, the scan pattern is a raster scan. Some such embodiments of the apparatus further include a control mechanism that receives user input and based on the user input automatically controls a width and a length of the scan pattern. In some such embodiments, the apparatus further includes a temperature sensor and a timing device operatively coupled to control the laser device such that a predetermined temperature of the first area of tissue is achieved for a predetermined period of time. Some embodiments further include an endoscopic mechanism operatively coupled to receive laser radiation from the laser device and configured to deliver the laser radiation to a specific location internal to the animal. Some embodiments of the apparatus further include an imaging device configured to obtain an image of at least a portion of a subject; a scanner mechanism configured to scan a beam of the laser radiation in a scan pattern across a first area of tissue to be conditioned; an imaging-processing device configured to identify a location of at least one fiducial on the subject and to control the scan pattern based at least in part on the identified location of the at least one fiducial; a temperature-sensing device configured to measure a temperature in the first area of tissue; and a controller operatively coupled to the temperature-sensing device, the scanner mechanism, and the laser device and configured to control an amount of laser radiation delivered to the first area based on the measured temperature of the first area. Some such embodiments of the apparatus further include having the scanner mechanism also configured to scan the laser beam in a scan pattern across a second area of tissue to be conditioned; the temperature-sensing device is also configured to measure a temperature in the second area of tissue; and the controller is also configured to control an amount of laser radiation delivered to the second area based on the measured temperature of the second area substantially independent of the amount of laser radiation delivered to the first area.

Some embodiments of the apparatus further include a masking apparatus having an aperture that controls a lateral extent of the dose of laser radiation. In some embodiments, the apparatus is controlled to raise a temperature of the first area of tissue of the animal to between 41 and 46 degrees C. for between 1 minute and 60 minutes. In some embodiments, the apparatus is controlled to raise a temperature of the tissue of the animal to between 43 and 44 degrees C. for between 5 minutes and 20 minutes. In some embodiments, the apparatus is controlled to limit the rate of temperature rise to be no more than a predetermined temperature change per unit time (e.g., to a rate of 5 degrees C. per minute). In some embodiments, the device is pre-set to achieve the most suitable tissue-temperature-time history for the conditioning effect (preconditioning or postconditioning). In some embodiments, the scanner moves a pulsed or continuous wave laser beam across the tissue region to achieve this temperature-time profile while stably maintaining the tissue temperature for each time. Thus, the scanning speed is sufficient to re-irradiate each tissue point faster than the time required for the tissue temperature to diffuse outside of this irradiated zone and thus decrease below the desired temperature value.

In some embodiments, the present invention provides a method that includes providing a source of laser radiation and selectively applying a therapeutically effective dose of the laser radiation to a first area of tissue of an animal, wherein the dose of laser radiation is controlled to improve a healing response to an injury of the animal.

In some embodiments, the preconditioning protocols of the present invention are used for such procedures as C-section birthing, tattoo removal, face lift or other cosmetic surgery of the face, plastic surgery such as breast implants or rhinoplasty, liposuction, open-heart surgery, spine surgery, neurosurgery involving craniotomy, tumor removal, endoscopic surgery such as prostatectomy, or minimally invasive spine surgery, and other planned surgical procedure of the skin, bones, or internal organs. In some embodiments, the postconditioning protocols of the present invention are used for such procedures as any of the procedures listed above for preconditioning, as well as procedures for thermal burn wounds, chemical burn wounds, incisional wounds such as a knife wound or laceration from an accident, excisional wounds such as bomb blast, shrapnel, burn, or skin graft, wounds following an amputation, wounds that were sutured, broken bones, trauma to the cochlea or inner ear, and general trauma to blood vessels, skin, bone, internal organs, brain, or other soft or hard tissues.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph 101 of the N-fold increase in Hsp70 versus exposure time for three different temperatures: 43, 44, and 45° C.

FIG. 1B is a graph 102 of the decrease in cell viability versus exposure time for three different temperatures: 43, 44, and 45° C.

FIG. 1C is a graph 103 that shows light-penetration depth for major soft tissue chromophores.

FIG. 1D is a macroscopic photograph 104 of laser-induced dermal wound in our transgenic Hsp70-luc-IRES-eGFP mouse model.

FIG. 1E is set of illustrations 105 that includes a bioluminescence image (BLI) 151 of laser-conditioned wound and a graph 152 showing BLI Quantification, which plots BLI versus lateral position across wound (mm).

FIG. 1F is an image depiction 106 representing a volume of skin 161 showing a model of hsp induction.

FIG. 1G is a block diagram of an output light coupler 107 according to some embodiments.

FIG. 2 is a graph 200 of tissue temperature versus time during exposure for three different fluences (measured in mJ/cm²).

FIG. 3A is a top-view image 301 of four areas of tissue each exposed to laser energy for different periods of time.

FIG. 3B is a graph 302 of blood flow versus number of days after exposure for four different exposure times.

FIG. 3C is a graph 303 of N-fold induction amounts of production of Hsp70 versus number of hours after exposure for four different exposure times at T_{H}E_{S}.

FIG. 3D is a graph 304 of N-fold induction amounts of production of Hsp70 versus number of hours after exposure for four different exposure times at T_{L}E_{L}.

FIG. 3E is a graph 305 of N-fold induction amounts of production of Hsp70 at 12 hours after exposure versus number of second of exposure at T_{H}E_{S}.

FIG. 3F is a graph 306 of N-fold induction amounts of production of Hsp70 at 12 hours after exposure versus number of second of exposure at T_{L}E_{L}.

FIG. 4A is a cross-section image 401 of an untreated wound at 12 hours.

FIG. 4B is a cross-section image 402 of a positive control at 12 hours.

FIG. 4C is a cross-section image 403 of a wound at 12 hours after exposure at T_{L}E_{L}.

FIG. 4D is a cross-section image 404 of a wound at 12 hours after exposure at T_{H}E_{S}.

FIG. 4E is a cross-section image 405 of an untreated wound at three days.

FIG. 4F is a cross-section image 406 of a positive control at three days.

FIG. 4G is a cross-section image 407 of a wound at three days after exposure at T_{L}E_{L}.

FIG. 4H is a cross-section image 408 of a wound at three days after exposure at T_{H}E_{S}.

FIG. 4i is a graph 409 of epidural hyperplasia at 12 hours and three days for the untreated wound, positive control, exposure at T_{L}E_{L}, and exposure at T_{H}E_{S}.

FIG. 5A is a cross-section image 501 of an untreated wound at three days.

FIG. 5B is a cross-section image 502 after three days of a wound with exposure at T_{L}E_{L}.

FIG. 5C is a cross-section image 503 of an untreated wound.

FIG. 5D is a cross-section image 504 of a wound after three days of an exposure at T_{L}E_{L}.

FIG. 5E is a cross-section image 505 of apoptosis in an untreated wound at three days.

FIG. 5F is a cross-section image 506 of apoptosis, after 3 days, of a wound with T_{L}E_{L} exposure.

FIG. 6A is a top-view image 601 of four areas of tissue the left ones 611 and 613 not pretreated, and the right-hand ones 612 and 614 pretreated with laser energy.

FIG. 6B is a graph 602 of N-fold induction amounts of production of Hsp70 in set of controls versus a set of laser pretreated 12 hours after exposure.

FIG. 7A is a cross-section image 701 of gomori trichrome for an untreated wound.

FIG. 7B is a cross-section image 702 of gomori trichrome for a preconditioned wound.

FIG. 7C is a cross-section image 703 of H&E for an untreated wound.

FIG. 7D is a cross-section image 704 of H&E for a preconditioned wound.

FIG. 7E is a graph 705 of epidural hyperplasia in a control versus a laser preconditioned wound.

FIG. 7F is a graph 706 of cell density in the wound bed in a control versus a laser preconditioned wound.

FIG. 8A is a graph 801 of wound strength as a percent increase in maximum load in controls versus laser-preconditioned wounds.

FIG. 8B is a graph 802 of wound strength as a percent increase in tensile stress in controls versus laser-preconditioned wounds.

FIG. 9 is a graph 901 of an Arrhenius damage analysis.

FIG. 10A is a block diagram of a high-level laser-preconditioning method 1001.

FIG. 10B is a block diagram of a high-level laser-preconditioning method 1002.

FIG. 10C is a block diagram of a high-level laser-preconditioning method 1003.

FIG. 11A is a block diagram of a more detailed laser-preconditioning method 1101.

FIG. 11B is a block diagram of a more detailed laser-preconditioning method 1102.

FIG. 11C is a block diagram of a more detailed laser-preconditioning method 1103.

FIG. 11D is a block diagram of a more detailed laser-preconditioning method 1104.

FIG. 12A is a block diagram of a controller computer display 1201.

FIG. 12B is a block diagram of a controller computer display 1201 at another point in time.

FIG. 12C is a block diagram of a controller system 1203 according to some embodiments.

FIG. 12D is a block diagram of a controller system 1203 at another point in time according to some embodiments of the present invention.

FIG. 13A is a block diagram of a tissue-conditioning laser handpiece system 1301.

FIG. 13B is a block diagram of a tissue-conditioning laser handpiece system 1302.

FIG. 13C is a block diagram of a tissue-conditioning laser handpiece system 1303.

FIG. 13D is a block diagram of battery-operated diode-laser-pumped rare-earth-doped fiber emitter tissue-conditioning handpiece system 1304.

FIG. 13E is a block structural diagram of a tissue-conditioning laser assembly 1305.

FIG. 13F is a block functional diagram of tissue-conditioning laser handpiece system 1305.

FIG. 14 is a block circuit diagram of tissue-conditioning laser handpiece system 1400.

FIG. 15 is a block diagram of focus-indicating tissue-conditioning laser handpiece system 1500.

FIG. 16 is a block diagram of surgery-inhibiting tissue-conditioning system 1601.

### DESCRIPTION OF EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

Terminology used herein: RAFT is an engineered skin equivalent model (for example, in some embodiments, an artificial skin model consisting of human keratinocytes in the epidermis and human fibroblasts and rat-tail collagen in the dermis, cultured using the floating collagen gel (RAFT) method); NHDF: normal human dermal fibroblast; NIH-3T3: a murine embryo fibroblast cell line; BAEC: bovine aortic endothelial cell; RPE: retinal pigment endothelial cell; NHEK: normal human epithelial keratinocytes; MDF: mouse dermal fibroblasts; IR: Infrared.

Wound repair is a complex coordinated sequence of overlapping biochemical and cellular events that result in the restoration of damaged tissue [1] (Davidson 1996). Under normal conditions of wound repair, the processes that result in healing of injured tissue follow a specific and well-defined time course. Various disorders, including diabetes, create conditions that impair the normal sequence of wound repair [2] (Braddock 1999) causing many diabetic patients to eventually develop chronic foot ulcers [3, 4] (Harris 1998, Frykberg 1999). Unsatisfactory mortality rates and a growing number of limb amputations, roughly 100,000 per year in the U.S.A. alone from diabetic foot ulcers, have sparked investigation into a variety of novel preventative and treatment strategies for enhancement of wound healing.

In one such method, researchers have shown that pre-treating cells or tissues with an initial mild thermal stimulus (often referred to as preconditioning), elicits a stress response that can serve to protect the tissue from subsequent lethal stresses [5-7] (Li 2003, Kim 2004, Topping 2001). There is evidence that heat-shock proteins are largely responsible for this preconditioning phenomenon. While a variety of tissue-heating methods have been used, preconditioning results from laser irradiation have been proven to be most effective by providing uniform and controlled temperature changes in cells and tissue. Moreover, laser-based approaches provide the ability to induce tissue heating in a non-contact fashion (unlike metal contact probes) which in turn is expected to mitigate the risk for infection and in some instances reduce pain.

In some embodiments, using an Aculight R1850 laser, the present inventors demonstrated that laser preconditioning tissue 6-12 hours prior to cutaneous injury (scalpel incision and laser ablation) significantly accelerated wound healing with greater tensile strength and improved cosmetic outcome. It was shown that Hsp70 is a useful biomarker for therapeutic efficacy of improved wound healing after tissue preconditioning [8] (Wilmink 2008). Using a novel molecular imaging approach where the promoter sequence for Hsp70 drives expression of an optically active reporter gene (luciferase and GFP) in a transgenic mouse, enabled real time quantification of the efficacy of tissue preconditioning in vivo without sacrifice of the model for histological analysis. The preconditioning protocol has been optimized for normal tissues prior to wound induction. In normal wound repair, Hsp70 is rapidly induced but in the chronic wound setting, Hsp70 is decreased [9] (McMurtry 1999). We hypothesize that a thermal-modulation protocol elevates the basal levels of heat-shock proteins and results in accelerated physiologic repair of the chronic wound. In some embodiments, the present invention provides a laser-based wound modulation device and protocol that can accelerate wound healing in diabetic patients suffering from chronic foot ulcers. In some embodiments, a laser is described and its efficacy validated in improving wound healing in a diabetic-animal model.

The light-pulse properties of certain lasers conventionally used to provide light used to stimulate nerves (e.g., the Capella R-1850 Infrared Nerve Stimulator available from Aculight Corporation) may also be nearly ideal for laser preconditioning to promote wound healing. The Capella device was implemented into experimental setups examining pretreatment of cells and in vivo tissues. A pretreatment protocol was optimized to accelerate cutaneous wound healing with improved wound strength and reduced scarring. Based on these results, in some embodiments, the existing architecture and light delivery scheme of the R-1850 Infrared Nerve Stimulator is modified to address a growing problem in the U.S.A. (diabetic foot ulcers). This forms a great example of modifying and applying the successful existing laser to address a markedly different, but similarly important clinical problem associated with diabetes. While some embodiments have optimized preconditioning protocols for normal (non-chronic) wounds, modifications to the existing Capella R-1850 are used in other embodiments for laser conditioning of tissue and a conditioning protocol to apply to existing foot ulcers and other conditions or wounds.

In some embodiments, the present invention provides a device that accelerates and improves healing of diabetic wounds in an animal model. Other embodiments provide a therapeutic modality aimed at treating chronic wounds (in particular foot ulcers) to improve quality of life for tens of thousands of patients and avoid debilitating and costly complications such as limb amputations. Some embodiments provide a device that achieves uniform tissue heating and subsequent tissue conditioning for improved wound healing.

Prevalence and Complications of Diabetic Foot Ulcers

The National Institute of Diabetes and Digestive and Kidney Diseases reports that there were 20.8 million diabetic patients in the U.S. in 2005, representing 7% of the population, with millions more at risk for developing the disease. The associated direct medical cost is considerable; estimated at $92 billion in 2002. Complications of diabetes include cardiovascular disease, atherosclerosis, diabetic retinopathy leading to blindness, high blood sugar causing kidney failure, and peripheral neuropathy leading to numbness. In addition to this list of serious medical complications, hyperglycemia also significantly impairs wound healing in the diabetic population, although the exact underlying cellular mechanisms remain unclear [10 ] (Schaffer 2007). The combination of neuropathy and impaired wound healing makes diabetics extremely vulnerable to chronic wound formation, specifically foot ulcers, which represent the most common wound care problem in the U.S.

Roughly 20 to 25% of the diabetic population, or 4 to 5 million patients, require treatment of foot ulcers at least once in their lifetime, making these lesions responsible for more hospitalizations than any other complication from diabetes. These wounds remain in a chronic inflammatory state while the body is unable to follow the normal wound healing response and therefore these lesions generally have difficulty healing. Treatment of foot infections requires attention to both local and systemic issues and coordinated clinical management [11, 12] (Lipsky 2004, Lipsky 2006). Therapeutic strategies used today include repeated debridement, offloading, and dressings, for lower-grade ulcers, and broad spectrum antibiotics and occasionally limited or complete amputation for higher-grade ulcers, requiring a team of health care workers from various specialties [13] (Eldor 2004). Approximately 100,000 limb amputations per year are a direct result of diabetic foot ulcers, while morbidity from these chronic wounds remains unacceptably high. In addition to this significant effect on quality of life and the devastating cost to society, the exact monetary cost of wound care in diabetic patients is difficult to estimate; however, the figure is undoubtedly in the billions. This figure will likely continue to rise given the major public-health issue that obesity and associated diabetes have become. The suboptimal patient outcome has lead scientists to explore new strategies for limiting development of foot ulcers, such as new off-loading techniques and dressings, artificial skin grafts, growth factors, and alternate methods of debridement [14] (Wu 2007). Despite these efforts, early recognition of etiological factors and prompt management remain the most effective strategy in the treatment of diabetic foot ulcers, although scientists continue to improve advanced wound-healing modalities [15] (Brem 2006). Thus, researchers continue to search for a local or systemic therapeutic strategy that can drastically accelerate wound healing in both early and late stages of diabetic wound formation.

Heat-Shock Proteins and Tissue Preconditioning for Accelerated Healing

The self-regulating process by which biological systems maintain stability and adjust to stressful conditions is termed homeostasis. Sub-lethal stimuli such as hyperthermia [16] (Morimoto 1996), desiccation, ATP depletion [17] (Kabakov 1997), and ischemia [18] (Richard 1996) can induce a stress response that alters homeostasis. The cellular response mechanism to thermal stress is exceedingly complex but consists in part of heat-shock proteins. Expression of heat-shock proteins is activated immediately after exposure to elevated temperatures and increases progressively over time with a maximum typically found approximately 12 hours after the thermal insult. Heat-shock proteins are found in all organisms and comprise a large family of proteins (for review see: [19-22] (Diller 2006, Mayer 2005, Young 2004, Young 2003)). These proteins play a large role in maintaining intracellular homeostasis by assisting in protein folding and mediating processes to protect the cell from further injury. One particular heat-shock protein, Hsp70, functions at key regulatory points in the control of apoptosis, thereby inhibiting cell death and promoting cell survival [23-26] (Samali 1996, Bowman 1997, Mosser 1997, Jaattela 1992-2). Hsp70 is highly inducible and can be upregulated (up to 15% of total cellular protein content) in the presence of cellular insults [27] (Pockley 2002). A basal level of Hsp70 exists in all cells in normal physiological conditions, but once denatured proteins are present, the heat-shock transcription factor HSF1 drives the production of excess Hsp70 to meet the demand necessary for repair [28] (Kim 1995). While some members of the hsp family (e.g., Hsp70 ) assist in the refolding of already unfolded proteins through ATP cycling [29, 30] (Frydman 2001, Nollen 1999), others (e.g., Hsp110 ) act as holding proteins that effectively shield other proteins from denaturing [31] (Oh 1997). Hsp90 is a holding protein and a folding protein [32, 33] (Duncan 2005, Yonehara 1996), while Hsp40 is a co-chaperone to Hsp70 by assisting it in refolding denatured proteins. Many other heat-shock proteins exist, each with a specific role in the response to stressful situations [34] (Samali 1998).

There is evidence that pre-treating cells or tissue with an initial mild thermal elevation elicits a stress response that can serve to protect the tissue from subsequent lethal stresses (called “preconditioning”) [5-7] (Li 2003, Kim 2004, Topping 2001). Stressors used to initiate preconditioning also include ischemia is pharmacological agents [7, 35-37] (Topping 2001, Minowada 1995, Morris 1996, Souil 2001); however, for the purposes of the present discussion preconditioning refers to pre-treatment using a heat stimulus. Preconditioned cells exhibit greater survivability than untreated cells when exposed to subsequent stresses [24] (Mosser 1997). In addition to in vitro cellular studies, in vivo tissue preconditioning has also been used. Since increased expression of Hsp70 is stimulated by heat-stress, it is hypothesized that this protein plays a significant role in the preconditioning phenomenon by increasing cellular protection.

Wound repair is a complex coordinated sequence of overlapping biochemical and cellular events that result in the restoration of damaged tissue [1] (Davidson 1996). Under normal conditions, the processes that result in wound healing follow a specific and well-defined time course. Various diseases, including diabetes, create conditions that impair the normal sequence of wound repair causing many of these patients to eventually develop chronic foot ulcers. In normal wound repair, expression of Hsp70 is rapidly induced, but in the chronic wound setting, Hsp70 is decreased [9] (McMurtry 1999). We hypothesize that a laser-conditioning protocol elevates the basal levels of heat-shock proteins and results in accelerated physiologic repair of the chronic wound.

Several studies have been conducted using preconditioning protocols to improve cutaneous-wound repair. Some studies using preconditioning for wound repair with electroheating probes to homogenously heat tissue [38] (Vigh 1997). Wound-healing conditions create a stressful environment for the cells involved in the regeneration process and are therefore postulated to influence the expression of heat-shock proteins [39] (Mehlen 1996). Hsp70 is rapidly induced at wound sites, and this induction has been shown to improve cutaneous-wound healing [38, 40] (Vigh 1997, Laplante 1998). Some very elegant work by Souil and coworkers showed up-regulation of Hsp70 in the skin by an 805-nm diode laser correlated with improved tissue regeneration and accelerated wound healing [37] (Souil 2001). Nevertheless, while the exact role of Hsp70 is still not clear, it is well established that hsp70 expression is correlated with thermal stress and as such can be used as a quantitative biomarker for the induction of the thermal conditioning (by preconditioning or postconditioning) response (Capon et al., 2008).

Potential Advantages of Infrared Lasers for Tissue Conditioning

Tissue-preconditioning protocols have been effectively incorporated into surgical procedures [41] (Snoeckx 2001), recovery of thermally injured tissues [42-44] (Seppa 2004, Baskaran 2001, Merchant 1998), protection of myocardial tissue from ischemia reperfusion injury, and cancer therapies [45, 46] (Rylander 2006, Wang 2004). Tissue-postconditioning protocols have recently also shown to be effective (Capon et al., 2008). A variety of stressors are capable of inducing heat-shock proteins, including chemicals, oxidative stress, desiccation, ischemia, and thermal changes. While any of these methods are potential options, chemicals, desiccation, and ischemia, and desiccation are difficult to control. One particular chemical, Bimoclomol, has been shown to effectively elevate Hsp70 levels in cells; however, this drug does not effectively penetrate the dermis and has significant side effects, limiting its practical clinical application [47, 48] (Hargitai 2003, Torok 2003). A delivery platform with the ability to supply precise dosimetry would aid in the optimization of preconditioning and postconditioning protocols, therefore a variety of contact and non-contact thermal delivery options have been used to carefully control temperature increases and thus heat-shock-protein induction. Contact-based heating techniques depend on thermal conduction/convection and include incubators, thermocyclers, water baths, and brass probes [49, 50] (O'Connell-Rodwell 2004, Wilmink 2006). Focused ultrasound, radio frequency, microwaves, and lasers are commonly used non-contact methods to deliver heat to tissue [45, 51-53] (Rylander 2006, Hoyte 2006, Ng 2004, Sherar 2001). Unlike other methods for inducing tissue heating or conditioning protocols, lasers allow fast heat deposition, uniform heat distribution, and therefore highly controlled tissue heating in time and space. In addition, laser energy can be delivered through optical fibers, allowing for extremely high-precision heating and the ability to deliver heat to internal organs (for potential preconditioning and postconditioning of organs) in a minimally invasive manner. While the same thermal induction and subsequent heat-shock-protein induction may be achieved by contact methods (e.g., using a metal probe) in particular when compared to long exposure, low irradiance laser exposures, the drawback of these conduction-based methods includes (a) slow, non-uniform, non-selective heating which is inherent to the conduction process; and perhaps more importantly, (b) the need for physical contact, which may be acceptable in a true precondition approach (where intact skin is preconditioned) but will be highly objectionable when treating/postconditioning existing ulcerating wounds. In contrast, laser light can be delivered in a non-contact manner, thus mitigating risks of infection and problematical circumstances associated with contacting an open wound.

Lasers are an integral tool in a wide variety of medical applications because their intrinsic properties allow for variable optical absorption from specific molecules (chromophores) at various wavelengths. The biological effects in tissue are governed by how well laser parameters are matched to the absorption characteristics of the target tissue. Selection of the proper laser for use in any given procedure is dependent upon matching the laser properties (wavelength, spot size, pulse duration, pulse energy, beam profile) with the tissue physical (heat capacity, thermal conductivity, amount of perfusion) and optical properties (absorption coefficient, scattering coefficient, anisotropy factor). Therefore the laser parameters must be chosen properly in order to optimize efficacy while minimizing unwanted side effects, such as thermal damage, for successful clinical outcomes. The volumetric tissue temperature increase can be highly controlled in time in a reliable and reproducible manner by carefully titrating the amount of light delivered to tissue (radiant energy per unit time per unit area (in J/sec-cm³ or W-cm³)) with the use of beam-shaping optics (control of irradiated area and spatial distribution of light), targeting a specific chromophore by using the most appropriate wavelength (control of irradiated depth), and measuring the temperature for each area and using feedback techniques to control the amounts of additional laser energy applied. In some embodiments, the tissue volume that is conditioned is precisely controlled by selecting a wavelength that has an effective tissue-penetration depth (governed by the optical properties in tissue including the light scattering coefficient, the light absorption coefficient, and the anisotropy factor) and controlling the irradiated area. The dosimetry of light (dependant upon the repetition rate, laser radiant exposure, and irradiation time for a pulsed laser and the laser irradiance and irradiation time for a continuous wave laser) determines the tissue's temperature-time profile.

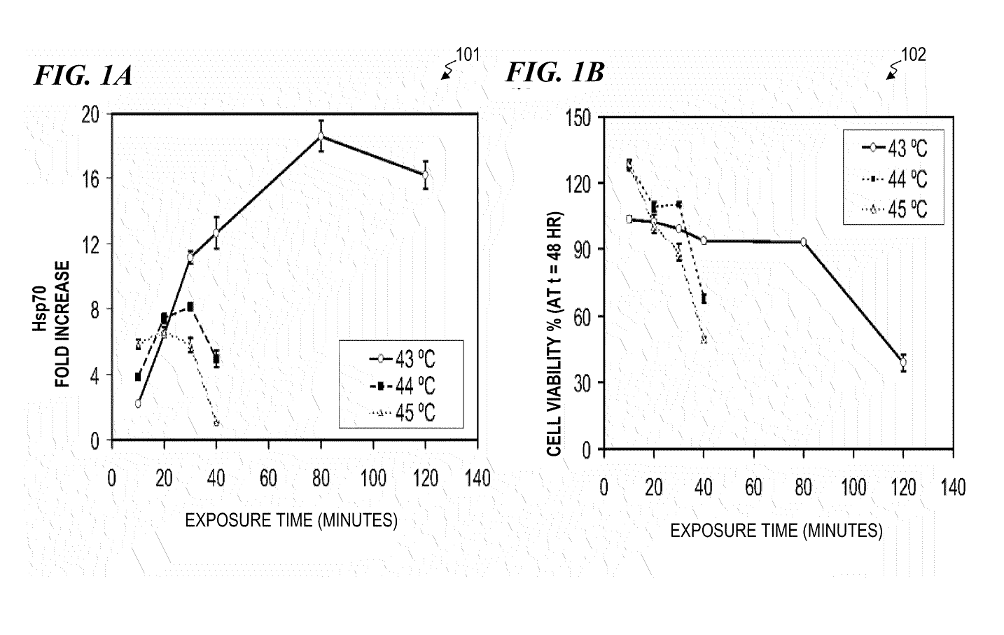

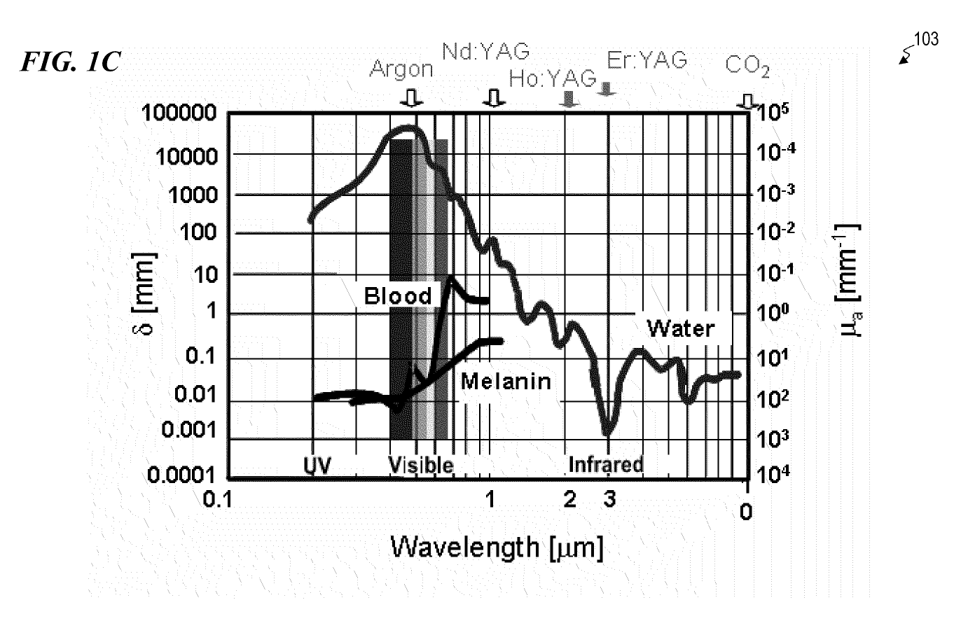

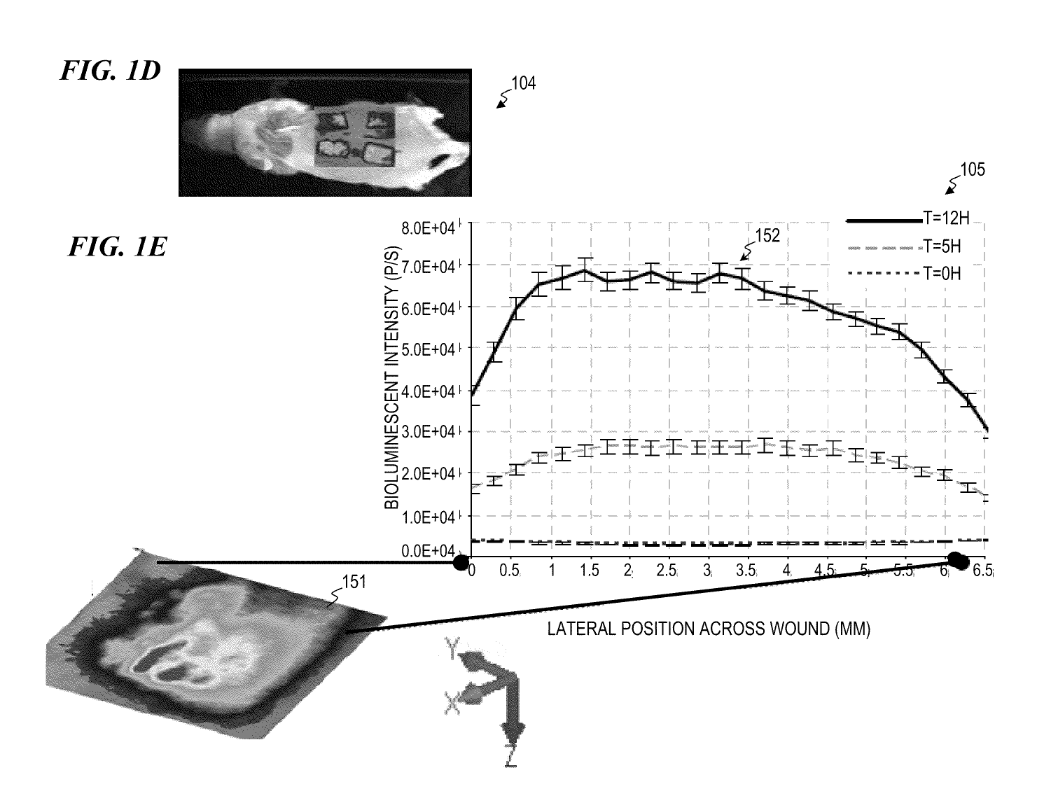

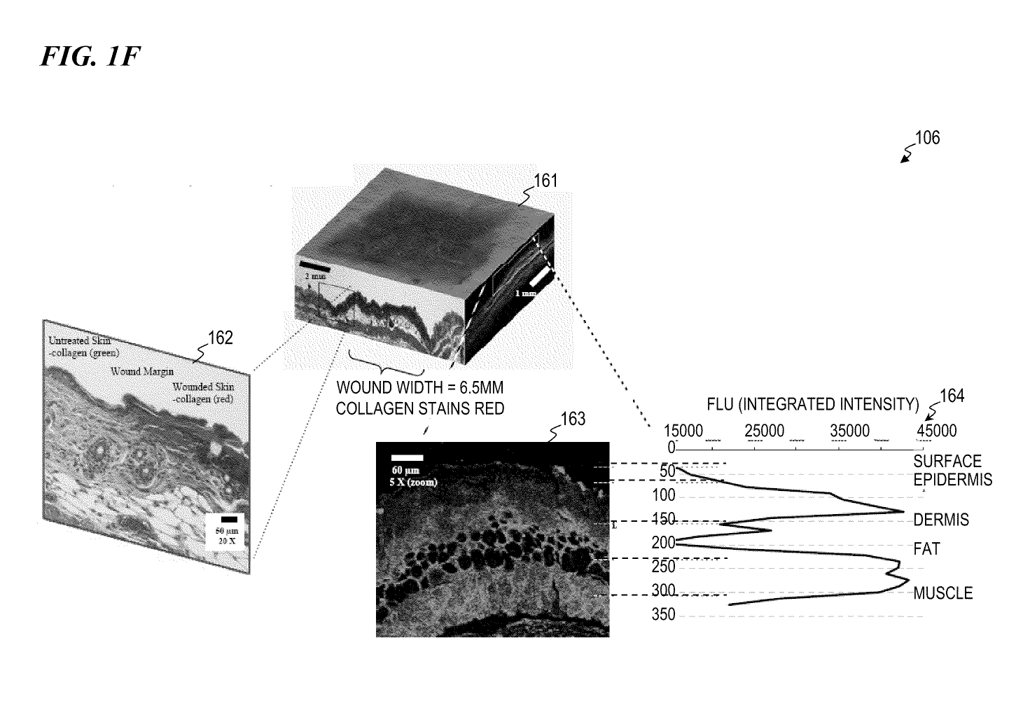

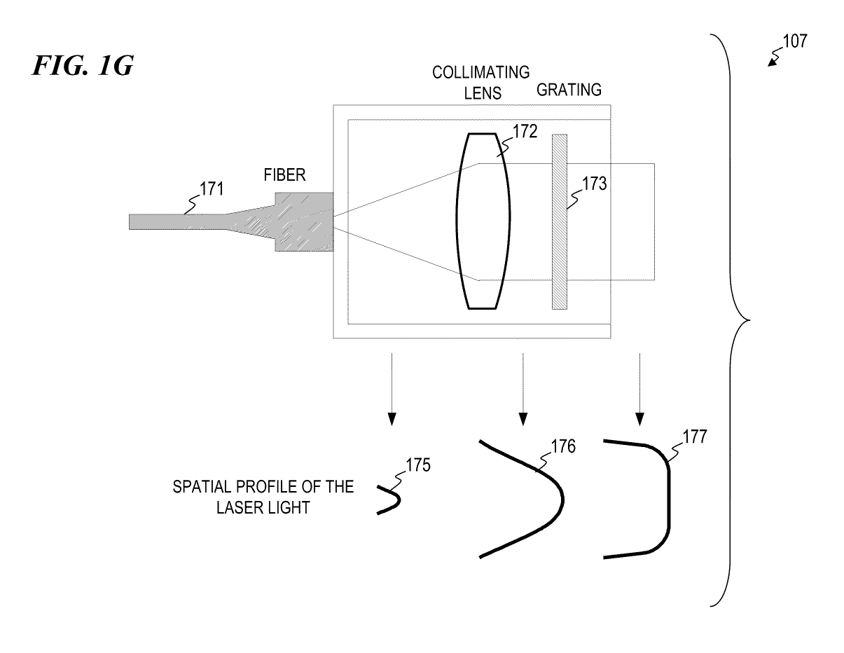

FIG. 1C is a graph 103 that shows light-penetration depth for the major soft tissue chromophores (water, blood, hemoglobin) as a function of wavelength across the ultraviolet, visible, and infrared portions of the electromagnetic spectrum.

In the infrared wavelengths of light (from 0.7 to 10 μm), water is the primary absorber in soft tissues, including skin (composed of 70-80% water). The tissue optical properties at a given wavelength dictate the optical-penetration depth, and in the case of the depth of preconditioned tissue. As seen in FIG. b1, tissue-penetration depths span four orders of magnitude (from 1 μm to 10 mm). By choosing a given wavelength, one can theoretically precisely control the photon distribution in tissue and subsequent volumetric heating.

In reality, specific laser sources are used to target exact penetration depths required for a particular application. In some embodiments, optical sources such as lasers, wavelength-conversion devices (for generating shorter wavelengths), optical parametric oscillators (OPOs, used for generating longer wavelengths) and the like operating at a wide variety of wavelengths in this regime (across the ultraviolet, visible, and infrared portions of the electromagnetic spectrum (100 nm-10 μm)) are available (e.g., such as are described in U.S. patent application Ser. Nos. 11/536,639, 11/484,358, 11/558,362, 11/420,754, 11/257,793, 11/567,740, 11/751,637, 11/682,234, 11/536,642, 12/018,193, 12/077,083, 12/050,937, 12/053,551 and 12/191,301, and U.S. Provisional Patent Applications 61/015,665 and 61/081,732, which are incorporated herein by reference). This capability allows generation of the desired penetration depth to match the particular goals and requirements of a medical procedure, such as cutaneous-heating requirements in the preconditioning procedures described herein. Some embodiments utilize a research-grade IR diode laser (Capella R-1850 available from Lockheed-Martin Aculight of 22121 20th Avenue S.E., Bothell, Wash. U.S.A. 98021) operating near 1.85 μm. This device was originally developed in Aculight's laboratory for safe, precise, and effective peripheral or cranial nerve stimulation. By using a diode that is tunable in wavelength (1.848-1.862 μm, for this particular laser embodiment), the laser-penetration depth can be controlled for maximum efficiency of tissue preconditioning based on the geometry and dimensions of the target tissue. Because the wavelength range covered by this laser spans a steep part of the water-absorption curve (different wavelengths have quite different penetration depths), the penetration depth of light from a tunable laser such as the Capella R-1850 laser is selectable (based on the wavelength to which the device is tuned) between 300-800 μm (0.3 to 0.8 mm) in soft tissue. Thus by optimizing laser parameters based on tissue morphology and optical properties, the irradiated tissue volume can be selectively targeted to match the goal of a particular procedure (for review see: [54] (Vogel 2003)). For the preliminary work in skin-wound healing, the light-penetration depth using the IR light from the Capella R-1850 allows for a desired penetration depth to suit the cutaneous-wound-healing application in a murine model, allowing effective and uniform heating of the entire dermis. In some embodiments, the laser parameters required for effective tissue preconditioning and postconditioning are determined by the type of tissue (its optical properties and morphology) and by the volume (depth and area) required to effectively create a temperature-time gradient required for the effect.

In simplified terms, laser energy absorbed by tissue is converted to thermal energy and the amount of energy absorbed per unit volume of tissue is directly related to the temperature rise in the tissue, which is dependent on the density (kg/m³) and the specific heat (J/kg K) of the irradiated material. The time course for this temperature increase through internal conversion of photon energy to heat can be assumed to be instantaneous. For longer exposures which are shown to be effective for preconditioning, heat transfer (conduction, convection and radiation) plays an important role and allows a controlled thermal equilibrium to be established.

In most scenarios of light-tissue interaction, optical energy converted to thermal energy can reversibly or irreversibly thermally damage cells and tissues. Biophysical markers such as vacuolization, hyperchromasia, protein denaturation (birefringence loss) are typical signs of thermal damage [55] (Thomsen 1991). More subtle thermal effects are not as obvious and often are not acutely apparent, but they can cause proteins in the cell to denature, rendering them non-functional. In response, heat-shock proteins (hsp) can be upregulated in order to repair heat stress to the cell. Most mammalian cell lines initiate their heat-shock response at a temperature increase of at least 5-6° C. [16] (Morimoto 1996). Work by the inventors has shown that the expression kinetics of Hsp70 follows an Arrhenius-rate process similar to that described for pure biophysical manifestations of thermal damage (i.e., protein denaturation) [56] (Beckham 2004). Other studies indicate that Hsp70 together with isoforms of TGF-b may contribute to an improved wound healing response [37, 57-59] (Souil 2001, Cao 1999, Capon 2001, Martin 1997). Even though Hsp70 has been implicated as playing an integral role in preconditioning, the desired degree of initial mild stressing still is unclear, as is the role and relative importance of the other heat-shock proteins. Some groups argue that a mild stress is sufficient to induce hsp expression [60] (Marber 1995), while other researchers believe a more severe stress is required for effective preconditioning [7] (Topping 2001). In all likelihood, a balance between hsp expression (induced by reversible intracellular protein denaturation) and induced irreversible damage must be met in order to bring about effective protective measures which minimize the necrotic cell death and apoptosis.

Given the necessary temperature required for induction of heat-shock proteins, the amount of laser energy (J/cm²) and irradiation time required to achieve this uniform temperature distribution are calculated and subsequently experimentally measured. As shown in our preliminary results, the laser-irradiation protocol for effective preconditioning (based on the required temperature rise in space and time) has been shown for laser pretreatment prior to normal tissue injury. Some embodiments use these results as a starting point for demonstrating the ability to accelerate healing in chronic diabetic wounds. Furthermore, hsp70 expression levels, measured with bioluminescent imaging (BLI) as a surrogate biomarker, allow a high throughput of parametric studies to optimize a laser-conditioning protocol over long time periods without the need for animal sacrifice and histological analysis.

One research infrared nerve stimulator (INS), the Capella R-1850, provides the neuroscientist researcher with a stand-alone nerve stimulator providing improved stimulation selectivity, no electrical artifact, and non-contact operation. While the Capella R-1850 was developed for a nerve-stimulation project, the laser wavelength proved to be beneficial in skin-preconditioning experiments conducted at the Vanderbilt Biomedical Optics lab. The R-1850 has many of the desirable features for a tissue-conditioning device and therefore provides an excellent framework from which to optimize the most appropriate laser-device characteristics, such as the most-appropriate wavelength.

Animal Models of Wound Repair

Much work has been done on advancing the accuracy of animal models for human wound repair. A broad review of available animal model strengths and weaknesses has been written by one of the inventors (Davidson), specifically citing the types of wounding protocols and their analyses of applicability to wound types. Murine models (which dominate in this area due to availability of numerous genetically modified phenotypes, optimized cost, per-diem maintenance costs, applicability and previous research data availability) have expressed certain biological conditions that make them more readily applicable to acute-wound repair than to repair of chronic wounds. Indeed, Davidson cites great difficulty in modeling the chronic wound due to the variable combinatory biological stresses that lead to its induction. Due to wound-healing mechanisms and the resultant increase in repair tissue volume, chronic-wound histological and biological parameters are more accurately modeled by full-thickness excisional wounding (i.e., removal of a significant volume of tissue and the filling of the void), as opposed to incisional wounds used in many studies. Excisional wounds are usually created with a biopsy punch, and subsequent healing rates are monitored as a function of wound volume or cross-sectional area, extent of reepithelialization, histological organization, and biochemical collagen content. Despite all the seeming benefits of this wound model, the loosely attached skin of rodents leads to several characteristics which are often seen as undesirable in endpoint monitoring. Contraction is defined as a phenomenon predominantly seen in rodent and rabbit models, in which the excision of loose skin results in rapid shrinkage of the surrounding skin in a presumably adaptive fashion. The measure of contraction is noninvasive and indirect, and in a chronic wound situation, its measure may be advantageous rather than deleterious, as it could give important time-scale data of impaired healing while minimizing the number of necessary animal sacrifices. The other major concern in excisional wounding is related to obesity, which is especially prevalent in diabetic murine models. The dermal fat deposits in obese mice affect the normal mechanisms of contracture, creating their own impedance to healing, which effects the accuracy of impaired healing endpoints [61] (Davidson 1998).

Besides the wounding protocol itself, much work has also been done to mimic human wound types. Admittedly more difficult than the basic acute wound, the variable biology of chronic wounds creates several problems in the scientific accuracy of recreation and applicability to certain human equivalents. Impaired healing models for diabetes fall under two specific categories defined by the source of the diabetic state: chemical or genetic. In murine models, the chemical most commonly used to induce diabetes is streptozotocin (STZ). A dangerous but effective chemical, STZ induces rapid development of diabetes mellitus by selective destruction of the beta-cells in the pancreatic islets. The process takes only hours but mice must be fasted at least 12 hours prior to injection and the efficacy of the treatment depends entirely on the dosage protocol. Furthermore, yields of usable diabetic mice are typically <50% and the life span of mice after successful induction of diabetes is limited (several weeks). STZ itself can lead to deleterious effects on macrophage function (known to be part of the wound-healing mechanism) [61] (Davidson 1998). STZ diabetic mouse models have been shown to demonstrate decreased excisional wound closure, reduced granulation tissue formation, and reduced angiogenesis in previous studies [61] (Davidson 1998). Nevertheless, the lack of efficiency and specificity of the STZ injection protocol is a known liability as far as using this model for long-term effects of diabetes. Another commonly used genetically modified murine diabetic model known as the db/db mouse has a tendency towards severe obesity which in turn impedes biochemical and histological assays, while the closure of larger excisional wounds is doubly impeded by the actual state of obesity, and the resistance to skin contracture [61] (Davidson 1998).

Recently a new genetic murine model for diabetes mellitus, dubbed the Akita mouse, was developed that shows no signs of obesity, and thus may be used to better study biochemical and histological endpoints in secondary effects of diabetes. The Akita mouse shows all the model accuracy advantages of early age onset, insulin secretion impairment, decreased active beta cell numbers, and a 50% survival time of 305 days [62] (Yoshioka 1997). In this mouse, a mutation of the Ins II gene at the Mody 4 locus leads to an autosomal dominant mutation that displays significantly higher mean morning blood glucose levels (via ANOVA) compared to unaffected mice as early as 7 weeks [62] (Yoshioka 1997). Due primarily to its non-obese characteristics, the Akita mouse has been commercially developed and is now publicly available through The Jackson Labs (Stock No. 002207). This model is intended for study of insulin-dependent diabetes mellitus with severe hyperglycemia, further indicating that the model is also accurate for study of diabetic effects on growth factors [63] (Akita Mouse Datasheet 2008). Several in-depth studies of secondary effects of diabetes by Breyer have used the Akita mouse to model diabetic nephropathies with notable success [64] (Breyer 2004). In a study by Gyurko et al, chronic hyperglycemia in Akita mice showed decreased leukocyte function and increased inflammation. They speculate the superoxide production of mitochondria in chronic hyperglycemia contributes to the endothelial damage essential to the long-term complications of diabetes. The demonstration of accurate modeling of these effects related to diabetic wound-healing impairment strongly supports the use of the Akita mouse model in diabetic chronic wound healing [65] (Gyurko 2006). Hence for some embodiments of a process to determine optimal laser tissue-conditioning protocols, and to determine the efficacy of laser conditioning of incisional and excisional wounds, the Akita mouse model is used.

Thermal Preconditioning Improves Cutaneous-Wound Healing

FIG. 1D is a macroscopic photograph 104 of laser-induced dermal wound in our transgenic Hsp70-luc-IRES-eGFP mouse model (at irradiances above the ablation threshold)).

FIG. 1E is a set of illustrations 105 that includes a bioluminescence image (BLI) 151 of laser-conditioned wound and a graph 152 showing BLI Quantification, which plots BLI versus lateral position across wound (mm).

FIG. 1F is an image depiction 106 representing a volume of skin 161 showing fluorescent microscopy of Hsp70-eGFP signal at 12 hours (5× magnification, bar=150 μm, where a 10-μm-thick cross-section slice of laser damaged dermis was imaged with Zeiss inverted LSM510 Confocal Microscope. Imaging mode: laser scanning fluorescence and DIC (Nomarski), 3-D “Z-series”, and time-series. Fluorescent filter 488 nm. Image 163 and graph 164 show Fluorescence Imaging Depth Quantification. The Hsp70-eGFP signal can be measured in the z-direction using confocal fluorescent imaging.

Image 161 shows immunohistochemistry of high-intensity laser-wounded skin. Gomori trichrome (green) taken at 2× magnification, bar=2 mm. Untreated collagen stains green and denatured collagen stains red. Image 162 shows the wound margin with a 20× magnification of the wound-margin image in image 161. Untreated collagen stains green and denatured collagen stains red.

The Vanderbilt Biomedical Optics lab has worked for over a decade to understand the cellular mechanisms of laser thermal effects. Recently they have found that preconditioning, optimized using molecular imaging approaches, provides an effective strategy to accelerate wound healing. It was shown that in vivo tissue preconditioning prior to incisional wounds leads to accelerated wound healing and improved cosmetic appearance of scars. Preliminary results show that appropriate laser exposure durations with the Aculight Capella R-1850 can induce cell proliferation with minimal cell apoptosis, focus cellular repair efforts, and increase wound healing strength and cosmetic appearance.

It has been shown that mild thermal effects result in a generic heat-shock response in cells and tissues. Further evidence has shown that Hsp70 is induced by this process and Hsp70 expression is correlated with wound repair. The Vanderbilt Biomedical Optics lab has demonstrated that cells equipped with photonic reporter gene systems can provide a continuous indicator of the cellular response to thermal stress [49, 66] (O'Connell-Rodwell 2004, Wilmink 2006). In these systems, expression of the luciferase (luc) transgene is directed by the promoter of the Hsp70A1 gene. Luciferase activity is non-invasively measured using bioluminescent imaging (BLI) methods, and provides a real-time and quantitative readout for Hsp70 transcriptional activation. Previously, the Vanderbilt Biomedical Optics lab has incorporated Hsp70-luc systems, both in cells and in skin equivalents, to assess the extent of sublethal cellular damage in the context of a laser-tissue interaction [56, 66] (Beckham 2004, Wilmink 2006), and to better understand Hsp70 expression kinetics [66] (Wilmink 2006). Subsequently a transgenic mouse model was generated with the Hsp70-luc-IRES-eGFP construct. The Vanderbilt Biomedical Optics lab has developed a number of optical imaging methods that can be used as tools for studying laser-tissue interactions and in particular to study the role of HSPs and their relation to preconditioning effects in cells and tissues (FIG. 1E and FIG. 1F). Such a method allows for data collection to assess the effectiveness of a laser conditioning protocol (correlated to BLI intensity and hsp expression levels) in the same animal over long time periods.

FIG. 1G is a block diagram of an output light coupler 107 according to some embodiments. In some embodiments, coupler 107 includes a fiber 171, collimating lens 172, and a grating 173 that are used to provide a collimated output beam of laser light. The collimated output beam avoids the difficulty of maintaining a focused beam at the correct distance needed to provide the proper energy density to reliably and repeatably obtain the desired temperature. Graph 175 shows the cross-sectional intensity profile of the beam as it leaves the fiber 171, graph 176 shows the cross-sectional intensity profile of the beam as it leaves the lens 172 and graph 177 shows the cross-sectional intensity profile of the beam as it leaves the coupler 107, showing an intensity with a so-called top-hat profile.

In the following sections, we detail how a conditioning device and protocol that can accelerate and enhance wound healing in impaired (diabetic) mouse model are developed.

A Prototype Fiber Coupled Diode Laser and Handpiece for Wound Healing Experiments.

In some embodiments, the design is based on a modified Capella nerve stimulator, due to its broad tunability of laser parameters, which can be modified to the most suitable parameter set for a clinical device. This system is a pulsed laser system with the following characteristics: tunable pulse width from 10 μsec-20 msec, repetition rate from 0.4-1000 Hz, maximum duty factor=10%, maximum optical pulse radiant exposure of 5 W through a standard optical fiber (200-600 μm). The device currently has features that are directly applicable to the tissue-conditioning-for-wound-healing application; such as air cooling, internal and external triggers, and tunable wavelength from 1.84-1.87 μm (300-1000 μm penetration depth).

For development, three elements are redesigned and modified to collect meaningful results for optimizing a protocol. The modified system then produces:
The beam output having a substantially uniform beam profile at the tissue (non-contact delivery) using a hand-held delivery probe/wand with a beam profile uniformity of better than +/−10%—which translates to a tissue temperature uniformity across the irradiated zone of significantly less than +/−0.5° C.
A tunable radiant exposure with pulsed or continuous wave (CW) output of up to 2.5 W/cm² at the tissue over each approximately 1-cm² area. This allows for an increase in tissue temperature that is 50% greater than the temperature increases shown to be most effective for preconditioning in the preliminary results (i.e., this provides a margin of 50% compared to the radiant exposures required for maximum hsp70 induction). In some embodiments, the stability of this output is better than +/−5%, providing a substantially constant and known absorption-driven tissue temperature increase.
The laser outputs a beam with a duty cycle that ranges from approximately 10% up to approximately 100% (i.e., continuous wave (CW)) at a wavelength of about 1.84 to 1.87 μm, which is obtained by modifying the platform to output a beam with a variable duty cycle of between about 10% duty cycle and a 100% duty cycle (i.e., the laser diode is constantly on), and a thermal-management redesign. This wavelength range is suitable to irradiate the entire depth of the murine-model skin thickness for effective laser conditioning in a mouse skin (total depth of the epidermis and dermis is less than 1-mm thick) wound-healing application. Thermal stability helps maintain a constant wavelength output (to control penetration depth), where the output wavelength of the diode is sensitive to the diode temperature (this is controlled to about +/−0.5° C. diode temperature, or a wavelength control of about +/−0.4 nm).

FIG. 1G shows a collimating lens and grating assembly used to provide a spatially uniform beam profile, in some embodiments.

Based on preliminary results, it has been found that using a uniform distribution of the light to the affected area is important, in some embodiments, for the effectiveness of laser conditioning. In some embodiments, a precise, uniform temperature rise in the tissue is important for effectively treating tissue, and can be accomplished with a uniform light distribution with a known and highly controlled photon density at the tissue surface. The output from a fiber-coupled diode-laser system naturally takes a Gaussian shape as the beam diverges from the output end of the fiber (or group of fibers). The desired uniform distribution of light can be implemented through a collimating lens set and a diffuser within a hand-held probe for tissue irradiation. In contrast to a diverging beam, where the photon density (fluence) changes with the distance to the tissue, collimation of the light is important to some embodiments of the present invention because the photon density will stay constant regardless of the distance from the output end of the probe to the tissue surface. The diffuser creates a “top-hat profile” or uniform distribution of light by using an array of diffraction patterns. In some embodiments, a square diffuser output is used, while in other embodiments, a circular diffuser output is used. In some embodiments, the square makes it easier to cover uniform patches of skin if multiple patches or large areas are required to be conditioned. In some embodiments, the circular pattern is useful for circular zones. In some embodiments, the collimating assembly of FIG. 1G is coupled to a standard 600-micron core (or greater diameter) fiber patch cord with a large angle divergence (0.37 NA). In some embodiments, the larger fiber size helps to efficiently couple the laser diode bar into the fiber as well as more uniformly distribute the light. In some embodiments, the collimating lens set is designed for a 1-cm aperture that provides a power density of at least 2.5 W/cm² at the tissue. In some embodiments, the uniformity of the beam profile is tested using a high-spatial- and high-temporal-resolution IR (thermal) camera to meet a specification (used in some embodiments) of better than +/−10%. This translates to a tissue-temperature uniformity across the irradiated zone of significantly better than (i.e., less than) +/−0.5° C., a number that is more than sufficient for an effective clinical preconditioning device (see preliminary results).

Some embodiments start with the same laser architecture that is used in Aculight's Capella 1850-R infrared nerve-stimulation research product. One primary difference between the Capella 1850-R and the tissue-conditioning device is that, in some embodiments, the preconditioning laser is capable of being run with a duty cycle in the range of about 10% up to about 100% (CW) rather than being limited to the 10% duty cycle used in the nerve-stimulation laser. As the duty cycle increases, (e.g., when using the 100% duty cycle) the laser will use a thermal cooling solution with sufficient capacity. For example, when running the diode in CW mode at full capacity with 60 amps current and 1.2 V (100% duty cycle), the diode requires a conservative 72-W cooling solution. The duty cycle and exposure time are chosen (i.e., tuned) to achieve a particular radiant exposure, measured in Watts/cm², to realize an effective clinical preconditioning (e.g., the preferred tunable radiant exposure when using a 100% duty cycle (CW) is up to 2.5 W/cm² at the tissue over a region of about a one-cm² spot size). In some embodiments, stability of this output is better than about +/−5%, providing a substantially constant and known absorption-driven tissue temperature increase. In some embodiments, stability across the spot is verified using an IR camera.

In addition, in some embodiments, the diode requires temperature control in order to tune the wavelength and maintain stability—which allows for fine control over penetration depth for conditioning tissues of various thicknesses. In some embodiments, this is accomplished by using a thermo-electric cooler (TEC) mounted on a large fan sink. Such heat sinks are commonly used for loads up to 100 W which should provide sufficient margin with the 72-W heat load used in some embodiments. In some embodiments, the heat sink maintains the temperature within +/−0.1 degree C. This helps maintain a constant wavelength output (to control penetration depth), where the output wavelength of the diode is sensitive to the diode temperature. To verify the stability of the emission spectrum for this IR light, some embodiments employ a mid-IR Optical Spectrum Analyzer (Ando AQ6315A) that readily measures the spectral content of incoming mid-IR light (2000 nm+/−300 nm). In some embodiments, a thermocouple is used to measure the operating temperature of the diode, which can be correlated to the wavelength output over the entire operating temperature range of the device (15-40° C.). In some embodiments, the diode temperature only needs to be controlled to within 0.5° C. which gives a wavelength sensitivity of approximately 0.4 nm.

Thus, in some embodiments, the adjustable parameters are laser power, laser wavelength, and aperture area. Some embodiments use a laser with a wavelength of 1.84-1.87 μm, with a selectably controllable output average power (delivered to the tissue) of up to 2.5 W in a uniform pattern covering 1 cm² (i.e., 2.5 W/cm²). These specifications provide a useful device for the calibration procedures that allows for adequate margins in key laser parameters to completely explore the parameter space for an optimized laser conditioning protocol.

Validate the Efficacy of the Tissue Conditioning Device in Terms of Improved and Accelerated Wound Healing in a Diabetic Animal Model.

In some embodiments, the efficacy and safety of this device for the intended use of wound conditioning is tested in a diabetic, difficult-to-heal wound. The experimental design consists of three specific sets of experiments.

A) With the prototyping laser and its delivery probe, Hsp70-luc (normal, non-diabetic) mice are used to determine levels of Hsp70 via BLI. Given that the inventors have shown luciferase expression (and therefore bioluminescence emission) to be an accurate surrogate marker for the induction of Hsp70 and by extension for preconditioning, this approach provides a rapid and high-throughput means to optimize the exact laser irradiation parameters (mainly irradiance and exposure duration). Mice are irradiated with the laser using a 1-cm-by-1-cm uniform spot and subsequently imaged using the IVIS 100 BLI system every 2 hours for the next 24 hours. After defining regions of interest in the image, quantitative measures of Hsp70 expression will be obtained for each of the combinations of laser parameters. In addition to optimizing these parameters for Hsp70 expression, histological analysis are performed to ensure tissue is not damaged (in some embodiments, five sets of laser parameters are determined since a wealth of previous data can be built upon). In some embodiments, as many as 4 lesions are produced on the dorsal side of one mouse. Statistical analysis based on prior knowledge of the variance in measurements like these, dictates a minimum sample size of n=5 for each laser parameter, in some embodiments.

Next the efficacy of laser conditioning in diabetic model is tested. For this, the Akita mouse model described in the background section is used in some embodiments. At the present time, this model is the most appropriate diabetic model in small rodents for studying the longer term effects of diabetes, including wound healing. Ultimately animal models that more closely mimic the human disease (Yucatan mini swine being the most obvious) may be used, but for this initial feasibility work, the Akita mouse model is adequate and provides a valid assessment model. Some embodiments use male mice ˜8-10 wks of age. Their blood sugar is checked regularly to make sure they are hyperglycemic/diabetic.

In some embodiments, a set of quantifiable milestones for positive results include at least a 50% increase in tensile strength at 7 days post laser (incisional wounds) and at least a 50% improvement in wound fill rate (excisional wounds) compared to controls.

In some embodiments, a device with the ability to supply precise dosimetry and real-time, non-invasive optimization of hsp70 expression yields improved preconditioning protocols (wherein the device can then be pre-set to output light patterns, intensities and durations that are based on a given skin type and other parameters of the environment in which the treatment is delivered such that a desired temperature, treatment area, and treatment duration are achieved based on a model derived from prior testing) and/or feedback mechanisms (wherein desired temperature, treatment area, and treatment duration are achieved based on measurements taken in real time from the patient during the conditioning treatment, in order to generate a cellular response that is correlated to the temperature and duration). That the cellular response to thermal stress can be serially monitored as expression of the luciferase (luc) transgene under control of the hsp70A1 promoter (O'Connell-Rodwell et al., 2004; Wilmink et al., 2006) was demonstrated using a transgenic-mouse model (hsp70A1-luc). The hsp70A1-luc system has been used to assess the extent of sublethal cellular damage in the context of a laser-tissue interaction (Beckham et al., 2004; O'Connell-Rodwell et al., Submitted; Wilmink et al., 2006), and to better understand hsp70-expression kinetics (Wilmink et al., 2006).

Some embodiments provide a fiber-coupled diode laser and hand piece for wound healing experiments and conditioning treatments. In some embodiments, the laser has a wavelength of 1.84-1.87 μm (1840 to 1870 nm), with an output average power (delivered to the tissue) of 2.5 W in a uniform square pattern covering 1 cm² (i.e. 2.5 W/cm²) with collimated light output from a handheld probe. In some embodiments, this light is delivered with uniform light distribution through a hand-held probe. Some embodiments then validate the efficacy of the tissue conditioning device in terms of improved and accelerated wound healing using a diabetic-animal model. Using a recently developed Akita diabetic mouse model, wound healing is investigated with and without laser conditioning of incisional and excisional wounds. Laser parameters are determined and optimized to induce maximum levels of Hsp70 that can be achieved without thermally denaturing tissue. The goals of some embodiments are to provide at least a 50% increase in tensile strength at 7 days post laser (incisional wounds) and at least a 50% improvement in wound fill rate (excisional wounds), as compared to controls.

In some embodiments, the present invention enables treatment of a significantly larger area with possible direct thermal feedback if necessary (in order to provide precise dosimetry) for an overall treatment protocol and medical device. It is anticipated that this device will immediately impact the diabetic patient population as well as have utility in areas of general and plastic surgery to improve wound healing complicated by diabetes and in the general population undergoing procedures that may benefit from accelerated healing.

In some embodiments, a mouse model (hsp70A1-luc) and thermal and optical imaging methods are used to develop and optimize a laser-preconditioning protocol for use on skin. Some specific goals of this study were to a) characterize the kinetics (magnitude, timing) of hsp70 expression in vitro and in vivo, b) select laser parameters that induce optimal Hsp70 levels while causing minimal tissue damage, and c) demonstrate the effectiveness of the protocols to enhance cutaneous-wound repair.

In Vitro hsp70-Expression Kinetics

FIG. 1A is a graph 101 of the N-fold increase in Hsp70 versus exposure time for three different temperatures: 43, 44, and 45° C. FIG. 1A displays hsp70-expression kinetics for mouse dermal fibroblasts (MDF) exposed to thermal-stress protocols at 43, 44, or 45° C. for 10, 20, 30, 40, 80, or 120 minutes. A maximal hsp70 expression, 18-fold greater than in the control cell cultures, occurred when the MDF were maintained at 43° C. for 80 minutes. At higher temperatures maximal hsp70 expression was blunted and it only reached 8-fold at 44° C. for 30 min and a 6-fold at 45° C. after 20 min. In the groups exposed to the briefest thermal stress (10 min exposure), hsp70 expression was linearly proportional with temperature.

In vitro Cell Viability Studies in Mouse Dermal Fibroblasts

FIG. 1B is a graph 102 of the decrease in cell viability versus exposure time for three different temperatures: 43, 44, and 45° C. Levels of hsp70 expression were markedly affected by cell viability as shown in FIG. 1B. At the lowest level of thermal stress (43° C.) cells maintained viability for the longest period (80 min) before showing a sharp decline, while cells that were exposed to incrementally higher thermal stress conditions at 44° C. or 45° C. showed precipitous declines after much briefer exposures of 20-40 min. Interestingly, cell viability data for MDFs exposed to very transient (10 min) thermal stresses at 44 or 45° C. showed enhanced numbers of viable cells compared to controls, suggesting that transient high temperature exposures may stimulate cell proliferation.

Temperature Calibrations for Laser Conditioning Protocols In Vivo

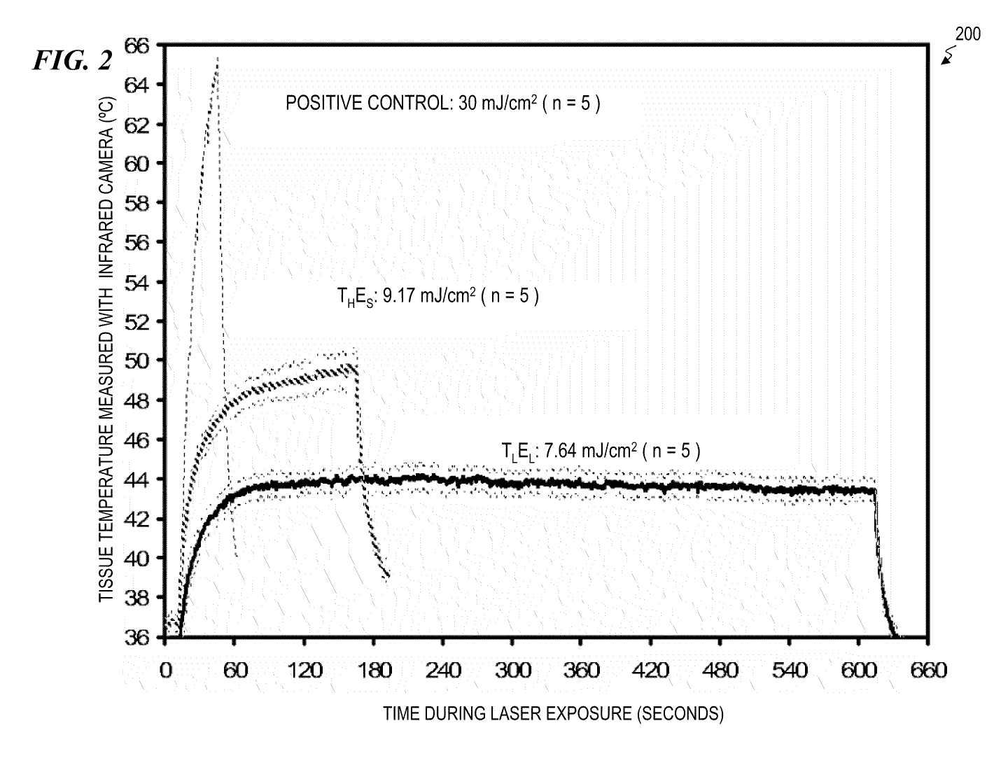

FIG. 2 is a graph 200 of tissue temperature versus time during exposure for three different fluences (measured in mJ/cm²). In some embodiments, a laser source is selected to test the effect of thermal preconditioning in vivo. Tissue temperatures were measured with an infrared camera. FIG. 2 presents the tissue temperatures induced during laser treatment for two laser preconditioning protocols and a positive control protocol (H=30 mJ/cm² for 30 s). The T_{H}E_{S} protocol (high fluence, short exposure) generated tissue temperatures ranging between 48 and 50° C. while the T_{L}E_{L} protocol (low fluence, long exposure) generated tissue temperatures between 43 and 44° C.

Visualization of In Vivo hsp70-Promoter Activity within Laser Treated Skin

Cell cultures or engineered skin equivalents lack many cell populations and essential cytokines needed for tissue repair, thus we utilized the hsp70A1-luc mouse model to assess the effects of thermal preconditioning in vivo (Beckham et al., 2004; Contag & Bachmann, 2002; Contag et al., 1997; O'Connell-Rodwell et al., Submitted; O'Connell-Rodwell et al., 2004; Wilmink et al., 2006). Bioluminescent imaging was used to verify that hsp70-promoter activity could be visualized on laser-treated mouse dorsum. We have previously shown that the bioluminescence signal correlates to the amount of Hsp70 protein and can therefore be used as a quantitative biomarker for hsp70 expression (Wilmink et al., 2006).

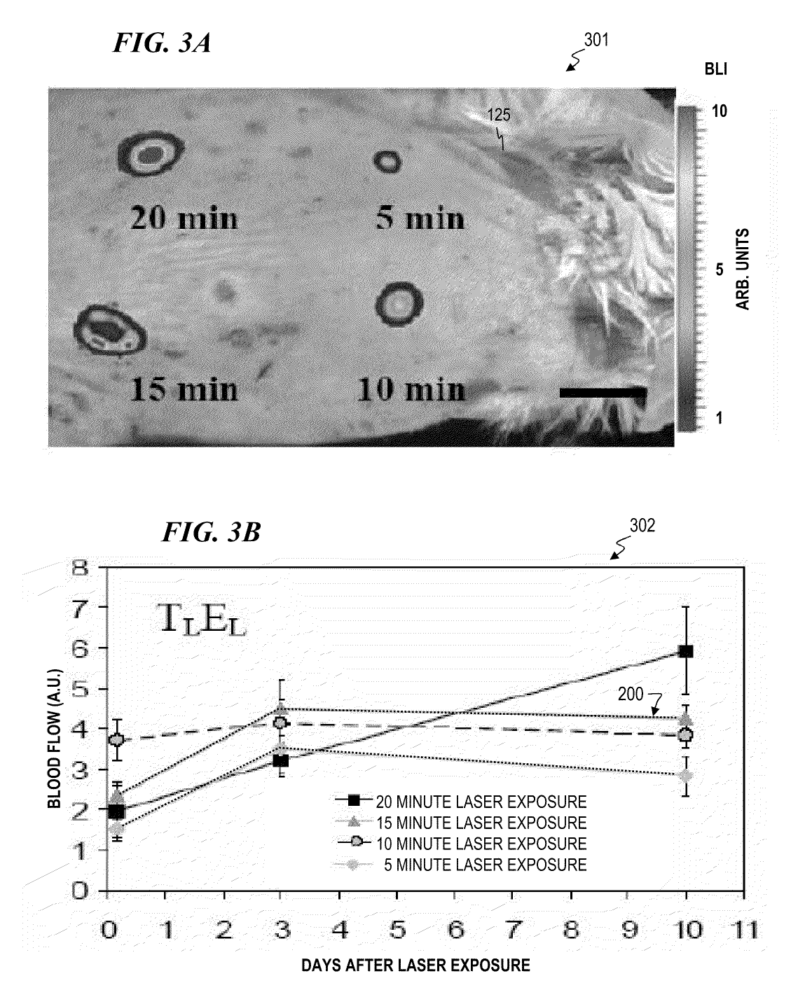

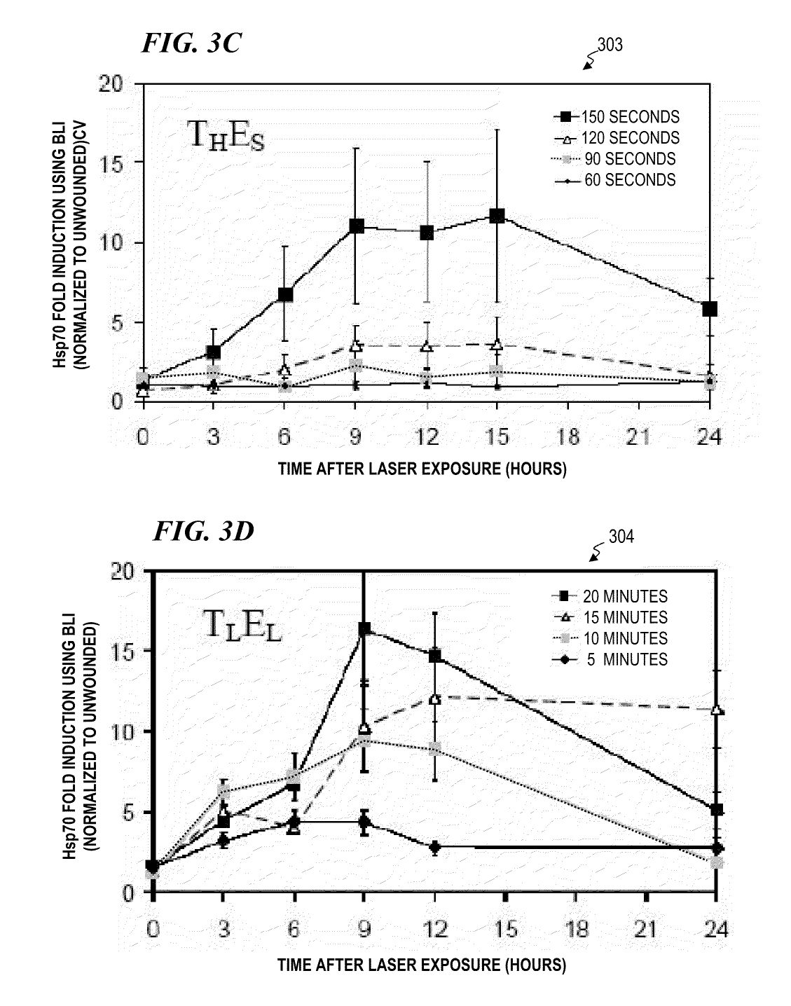

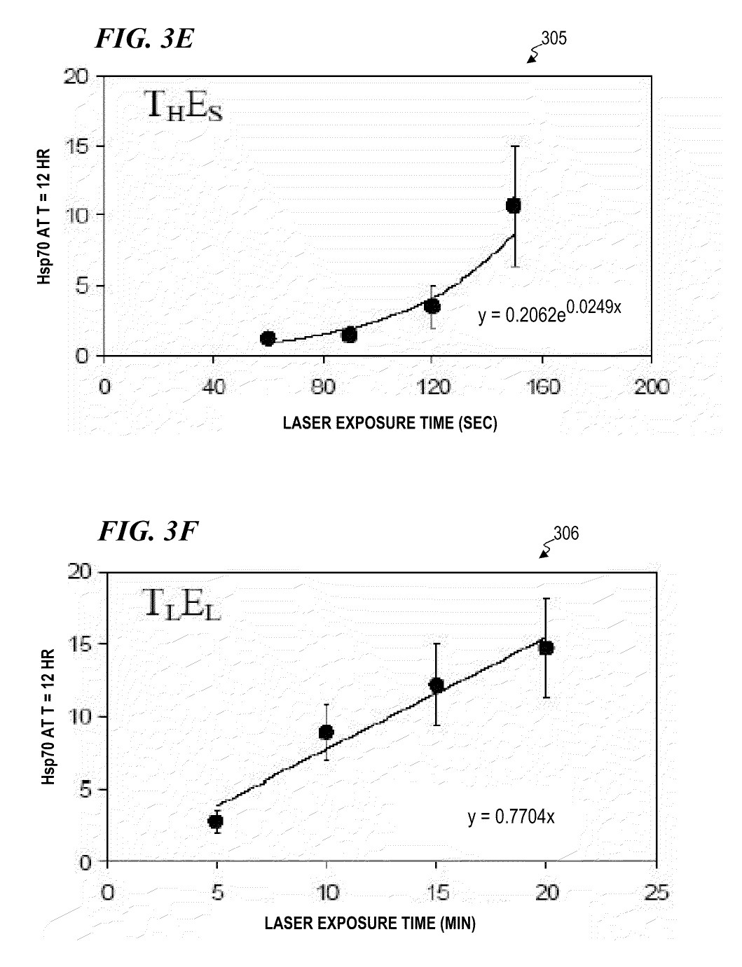

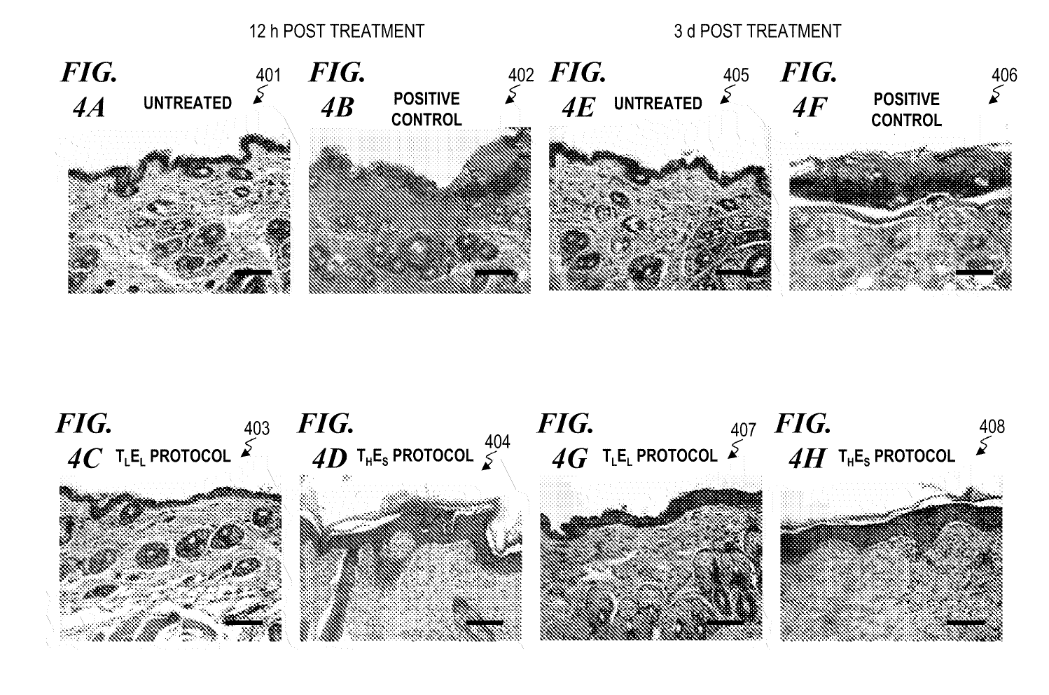

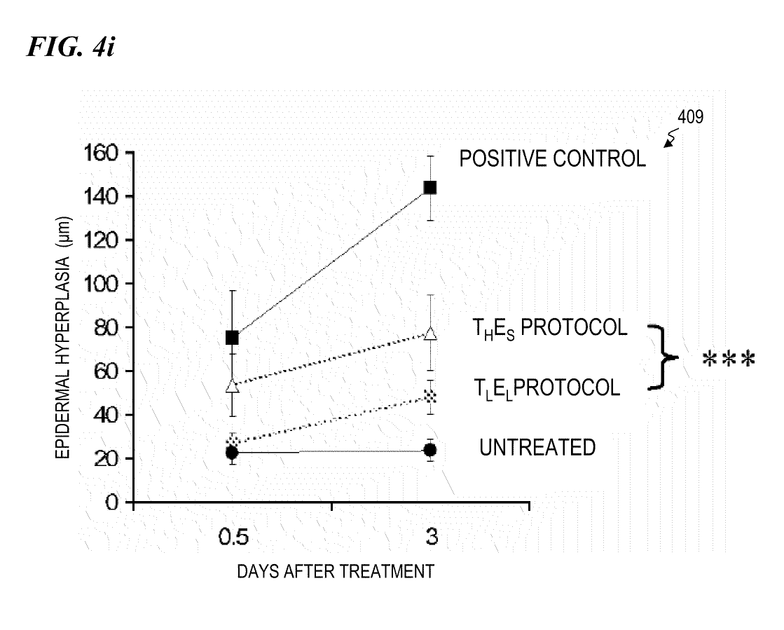

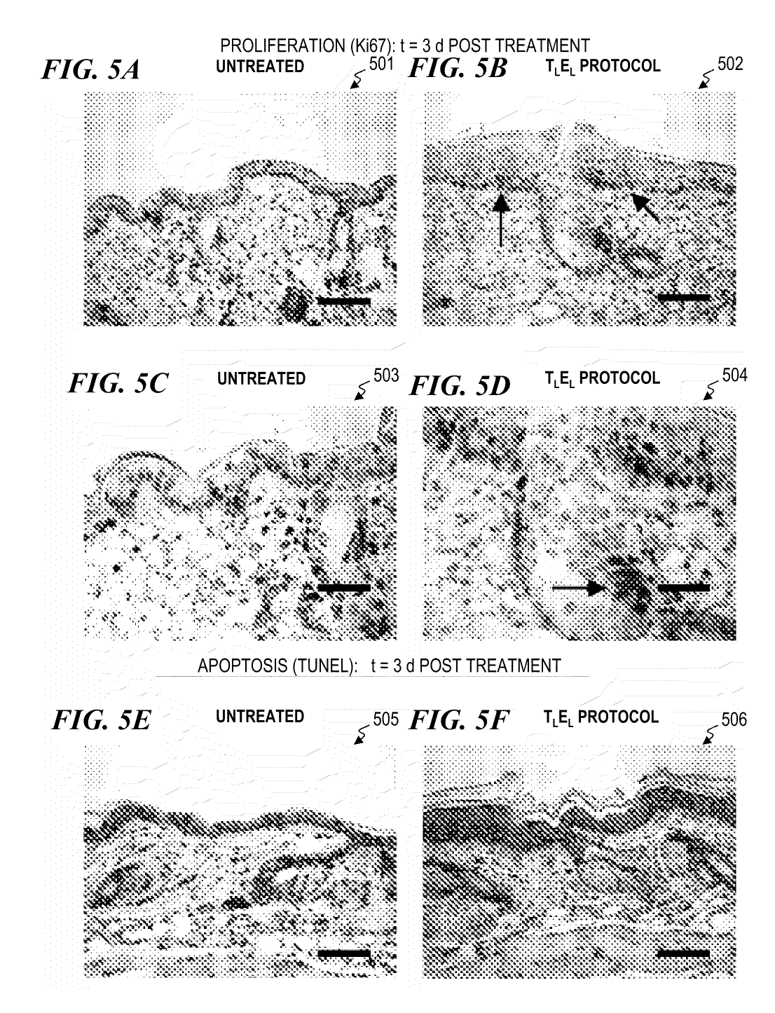

FIG. 3A is a top-view image 301 of four areas of tissue each exposed to laser energy for different periods of time. FIG. 3a presents a sample bioluminescent image of the dorsal surface of a transgenic mouse 12 hours after a laser preconditioning protocol (H=7.64 mJ/cm²). Bioluminescent emission (false colors) resulting from four laser treatments, using exposure durations of 5, 10, 15, and 20 minutes are visible on the dorsum of the mouse. The 20-minute exposures evoked greater hsp-protein levels as compared to shorter exposures.

Laser Preconditioning Increases Blood Flow

FIG. 3B is a graph 302 of blood flow versus number of days after exposure for four different exposure times. Since local hyperthermia has been demonstrated to increase blood flow (Song, 1984), we sought to examine if laser preconditioning protocols increased blood flow to the treated regions. Blood perfusion was measured 10 minutes, 3 days, and 10 days after laser preconditioning treatments using laser Doppler imaging. The highest perfusion rates, 6-fold greater than control skin, were measured at 10 days following a 20-minute exposure (FIG. 3B). The 10-minute laser exposure induced the highest flow that was 3.71-fold greater than controls. This level persisted for 10 days, while the 20-minute laser exposure showed a progressive increase in blood flow. Making the laser Doppler measurements in and by itself did not result in a measurable temperature rise (data not shown).

Optimization of In Vivo hsp70-Promoter Activity in Laser Treated Skin

FIG. 3C is a graph 303 of N-fold induction amounts of production of Hsp70 versus number of hours after exposure for four different exposure times at T_{H}E_{S}. Various exposure conditions were tested for each laser protocol to determine the relationship between exposure duration and hsp70-expression levels. For the T_{H}E_{S} protocol (9.17 mJ/cm²), exposure durations of 60, 90, 120, and 150 seconds were tested. Maximal hsp70 expression occurred nine to fifteen hours after laser exposure (FIG. 3C). Laser pre-treatments produced maximal hsp70-expression levels that were 11.65-fold greater than controls at an exposure duration of 150 seconds. The 120-second exposure induced 4 times less hsp70 expression than the 150-second exposure (p<0.05).

FIG. 3D is a graph 304 of N-fold induction amounts of production of Hsp70 versus number of hours after exposure for four different exposure times at T_{L}E_{L}. The T_{L}E_{L} protocol (H=7.64 mJ/cm²) at exposure durations of 5, 10, 15, and 20 min produced markedly different profiles compared to the T_{H}E_{S} protocol. The amount of hsp70 expression increased linearly with increasing durations of exposure, and a maximum N-fold induction level of hsp70-expression of a 17-fold induction was achieved after a 20-minute exposure (FIG. 3D).

FIG. 3E is a graph 305 of N-fold induction amounts of production of Hsp70 at 12 hours after exposure versus number of second of exposure at T_{H}E_{S}.

FIG. 3F is a graph 306 of N-fold induction amounts of production of Hsp70 at 12 hours after exposure versus number of second of exposure at T_{L}E_{L}. FIG. 3E and FIG. 3F depict the hsp70 fold induction levels as a function of exposure time. In the T_{H}E_{S} protocol the hsp70 levels increased exponentially with laser exposure time, while the hsp70 levels increased linearly with laser exposure time in the T_{L}E_{L} protocol. These optimization data suggest that the T_{L}E_{L} protocol may provide a more consistent and reliable induction of hsp70 levels.

Histologic Studies to Evaluate Preconditioning Protocols

FIG. 4A is a cross-section image 401 of an untreated area at 12 hours.

FIG. 4B is a cross-section image 402 of a positive control at 12 hours.

FIG. 4C is a cross-section image 403 of a wound at 12 hours after exposure at T_{L}E_{L}.

FIG. 4D is a cross-section image 404 of a wound at 12 hours after exposure at T_{H}E_{S}.

FIG. 4E is a cross-section image 405 of an untreated area at 3 days.

FIG. 4F is a cross-section image 406 of a positive control at 3 days.

FIG. 4G is a cross-section image 407 of a wound at 3 days after exposure at T_{L}E_{L}.

FIG. 4H is a cross-section image 408 of a wound at 3 days after exposure at T_{H}E_{S}.

Immunohistologic studies were conducted in preconditioned mouse skin to evaluate histological damage, cellular proliferation, and apoptosis. FIGS. 4A-4D and 4E-4H reveal marked differences in the histological characteristics among the four treatment groups at 12 hours and 3 days. Control tissues are illustrated in FIG. 4A and FIG. 4E show healthy intact mouse skin. The lethal laser protocol (positive control; FIG. 4B and FIG. 4F) produced tissue damage to a depth of 150 μm. Tissue treated with the T_{L}E_{L} preconditioning protocol showed virtually no damage evident in only minor alterations to the stratum corneum (FIG. 4C and FIG. 4G). In contrast, the T_{H}E_{S} protocol induced mild damage as evidenced by epidermal hyperplasia and a modest inflammatory infiltrate in the papillary dermis (FIG. 4D). FIGS. 4E-4H also reveal marked differences in histological characteristics among the four treatment groups. The most noticeable difference 3 d post-treatment was the degree of epidermal hyperplasia.

FIG. 4i is a graph 409 of epidural hyperplasia at 12 hours and 3 days for the untreated wound, positive control, exposure at T_{L}E_{L}, and exposure at T_{H}E_{S}. The thickness of the epidermis hyperplasia was plotted versus time for each treatment group. The data show that the T_{L}E_{L} protocol induced less epidermal hyperplasia than the T_{H}E_{S} protocol. Pronounced epidermal hyperplasia, a characteristic of the intermediate phase of wound repair, suggesting that the T_{L}E_{L} protocol produced less epidermal injury than the T_{H}E_{S} protocol (Florin et al., 2006).

FIG. 5A is a cross-section image 501 of an untreated wound at 3 days.

FIG. 5B is a cross-section image 502 after 3 days of a wound with exposure at T_{L}E_{L}.

FIG. 5C is a cross-section image 503 of an untreated wound.

FIG. 5D is a cross-section image 504 of a wound three days after a treatment with exposure at T_{L}E_{L}.

FIG. 5E is a cross-section image 505 of apoptosis in an untreated wound at 3 days.

FIG. 5F is a cross-section image 505 of apoptosis after 3 days of a wound with exposure at T_{L}E_{L}. The extent of cellular proliferation was examined using a Ki67 immunodetection (FIGS. 5A-5D), and apoptosis was evaluated with a TUNEL stain in tissues from the T_{L}E_{L} protocol (FIG. 5E and FIG. 5F). There was substantial proliferation in the basal cells and in the bulge region around the hair follicle in the preconditioned tissues (FIG. 5A and FIG. 5C). While the control tissue (untreated) showed few basal cells actively replicating or proliferating (FIG. 5B and FIG. 5D). Laser preconditioned tissues did not show apoptotic activity (FIG. 5E and FIG. 5F). Caspase-3 antibody stains were also conducted and also show minimal evidence of apoptosis (data not shown).

Laser Preconditioning Protocols can Manipulate hsp70 Expression

The Vanderbilt Biomedical Optics lab recently applied these tools toward a protocol to enhance cutaneous-wound repair. Using the Aculight Capella R-1850 laser, the output penetration depth matches the dermal optical properties and thus provides an effective non-contact means of uniformly heating the murine dermis, we were able to increase hsp70 expression by at least a factor of 20. More importantly, when subjecting skin to a 10-minute exposure with this laser (resulting in a tissue temperature of roughly 43-44 degrees C.) 12 hours prior to inducing a full thickness scalpel wound, wound healing was markedly improved. Detailed experiments showed that this approach of relatively slow mild heating was more effective and safer in providing the preconditioning response than fast, more severe heating. Laser parameters used (and found to be optimal for the murine model skin with the existing Capella device) were wavelength=1.86 μm (penetration depth in soft tissue=400 μm), pulse duration=2 msec, repetition rate=50 Hz, and radiant exposure=7.64 mJ/cm², exposure duration 10 min, irradiance=382 mW/cm².

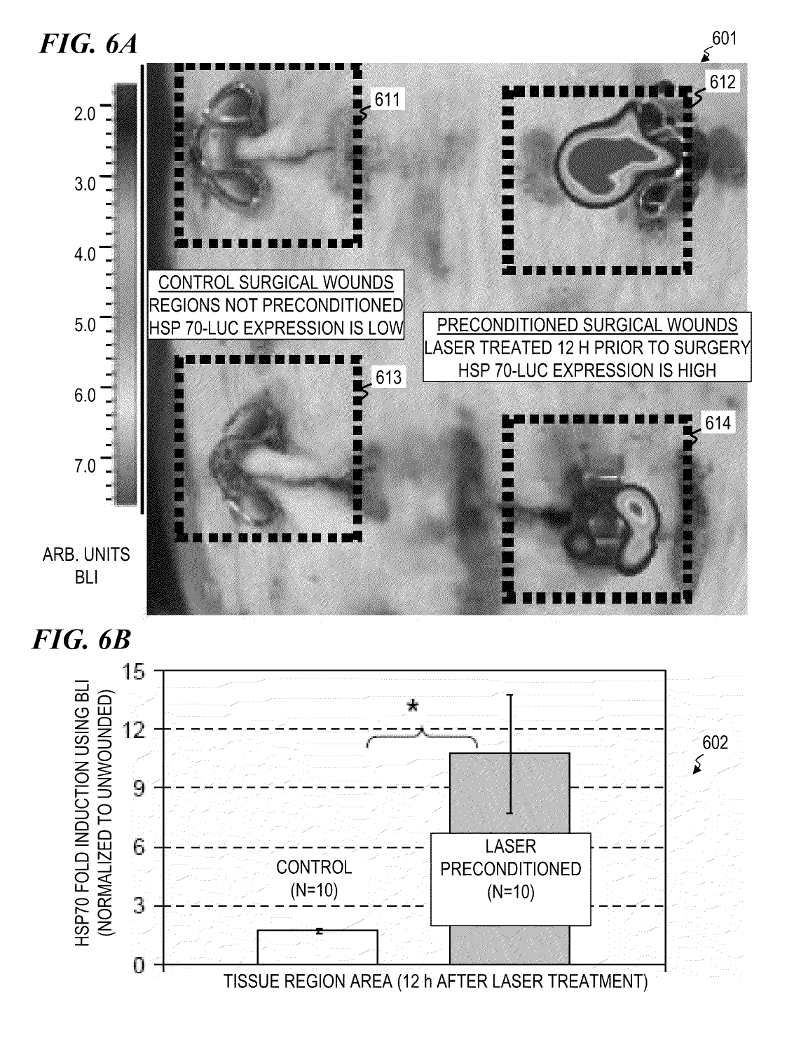

FIG. 6A is a top-view image 601 of four areas of tissue the left ones not pretreated, and the right-hand ones pretreated with laser energy. We investigated the effects of the T_{L}E_{L} laser preconditioning protocol on full thickness scalpel incisions. FIG. 6A presents a sample bioluminescent image of hsp70 promoter activity on a mouse with two preconditioned wounds (on the right) and two control scalpel incisions (on the left). The laser preconditioned wounds showed significantly higher hsp70 promoter activity. FIG. 6A shows laser manipulation of hsp70 expression before surgical wounding. (a) Bioluminescent representation of control wounds 611 and 613 (left) and laser pretreated surgical wounds 612 and 614 (right) at 12 hours post surgery.

FIG. 6B is a graph 602 of N-fold induction amounts of production of Hsp70 in set of controls versus a set of laser pretreated 12 hours after exposure. FIG. 6B shows the quantitative bioluminescent intensity for each wound 12 hours after preconditioning. Laser preconditioned areas had average of 10.75±3.03 fold-induction, while the control areas had averages of 1.72±0.15 fold-induction. A paired one-tailed student t-test indicated that the thermally preconditioned wounds had statistically significantly higher levels of hsp70 promoter activity (p<0.01). Ten regions were imaged for each condition and the mean Hsp70-fold induction was normalized to non-wounded control area of skin. The TLEL laser preconditioning protocol was used and student t-test indicated P<0.01.

FIG. 6A shows BLI of Hsp70 promoter activity on a mouse with two incisional wounds that were preconditioned (612 and 614) and two control scalpel incisions (611 and 613) with no laser pretreatment. This quantitatively demonstrates the bioluminescent intensity for each wound 12 hours following preconditioning, as shown in FIG. 6B. Clearly, the preconditioned wounds have much higher Hsp70 activity (demonstrated as a higher light emission profile that exceeds controls in BLI). Other experiments demonstrated that blood flow was also increased to the irradiated area, thus fulfilling an additional requirement for an effective preconditioning protocol.

Histologic Characterization of Incisional Healing Following Laser Preconditioning

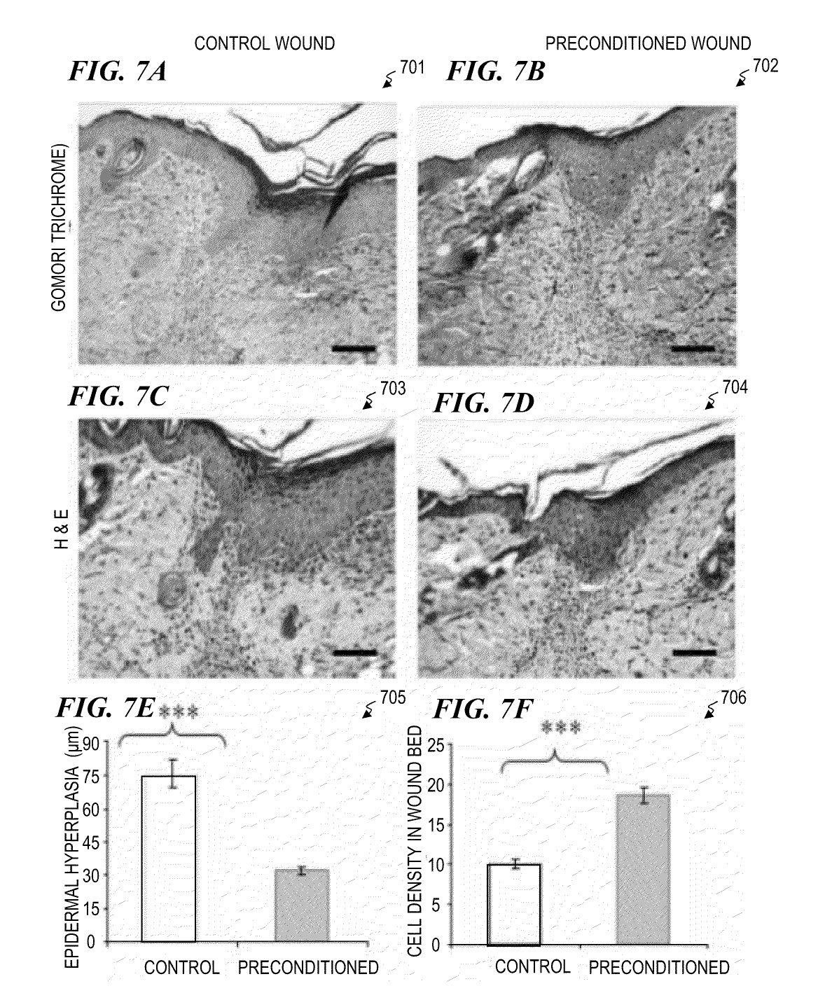

FIG. 7A is a cross-section image 701 of gomori trichrome for an untreated wound. To evaluate the affect that laser preconditioning (T_{L}E_{L}) has on early wound repair, qualitative patterns of collagen deposition were assessed after scalpel incision. In sections stained with Gomori trichrome, wounds that were not preconditioned show pale green staining in the adjacent tissue that is indicative of a wide degree of disruption to the collagen deposition patterns in the dermis immediately adjacent to the incisional site (FIG. 7A).

FIG. 7B is a cross-section image 702 of gomori trichrome for a preconditioned wound. Wounds preconditioned with the T_{L}E_{L} laser protocol show intense green staining indicative of normal non-disrupted collagen deposited patterns (FIG. 7B).

FIG. 7C is a cross-section image 703 of H&E for an untreated wound.

FIG. 7D is a cross-section image 704 of H&E for a preconditioned wound. Additional differences were evident between the non-preconditioned and preconditioned wounds within the surface epithelium (FIG. 7C and FIG. 7D).

FIG. 7E is a graph 705 of epidural hyperplasia in a control versus a laser preconditioned wound. Preconditioned wounds showed significantly less epidermal hyperplasia (31.9±1.7 μm) than control wounds (75.2±6.2 μm) (FIG. 7E) (p<0.001).

FIG. 7F is a graph 706 of cell density in the wound bed in a control versus a laser preconditioned wound. Granulation tissue in the preconditioned incisional wound beds also had a higher cell density (−19 cells/1000 μm²) compared to control wounds (−10 cells/1000 μm²) (FIG. 7F) (p<0.001).

FIG. 7A is a cross-section image 701 of gomori trichrome for an untreated wound. FIG. 7B is a cross-section image 702 of gomori trichrome for a preconditioned wound. FIG. 7C is a cross-section image 703 of H&E for an untreated wound. FIG. 7D is a cross-section image 704 of H&E for a preconditioned wound. FIG. 7E is a graph 705 of epidural hyperplasia in a control versus a laser preconditioned wound. FIG. 7F is a graph 706 of cell density in the wound bed in a control versus a laser preconditioned wound. These show histology of preconditioned and control wounds at five days. It can be seen that preconditioned wounds show reduced tissue disruption enhanced collagen deposition, and enhanced cellular density in the granulation tissue. Quantitative analysis in (e) and (f) demonstrates that preconditioned surgical wounds have significantly less epidermal hyperplasia than control samples with no preconditioning, an indicator of enhanced repair (***p<0.001) and increased cell density within each wound bed per 1000 μm² (***p<0.001).

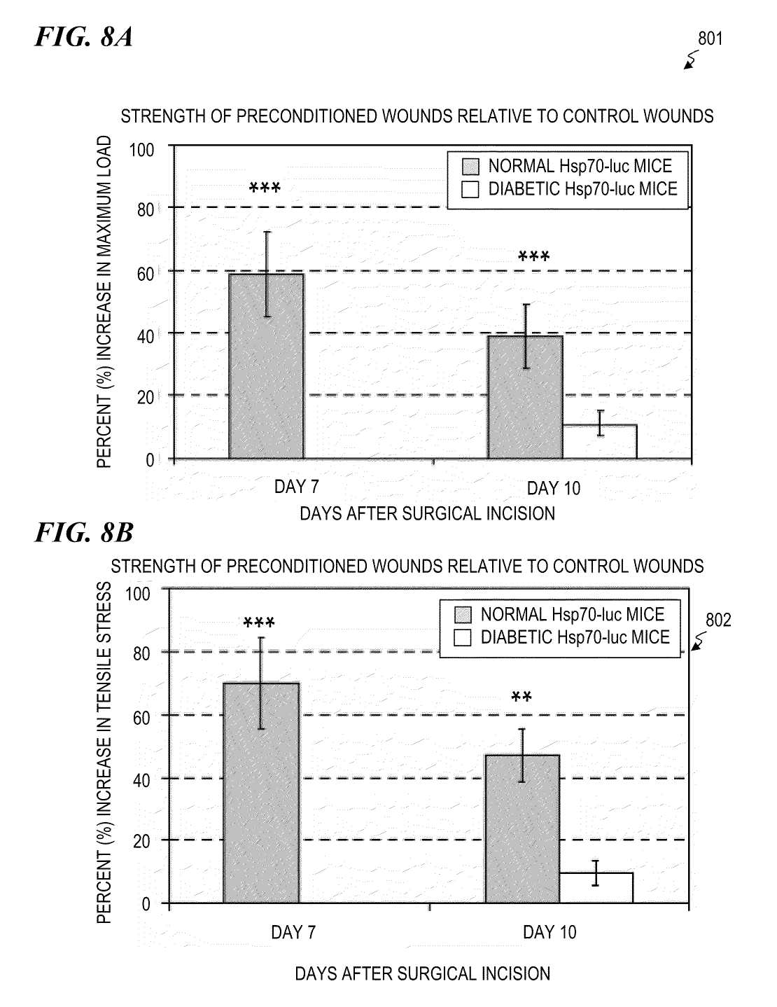

FIG. 8A and FIG. 8B show the effect of preconditioning on wound biomechanics. The maximum load and tensile strength of full-thickness wounded skin from mice (n=9) was measured at 7 and 10 days following injury. The maximum load in preconditioned incisions was 60% higher than controls 7 days following surgery and 40% higher than controls at day 10. The maximum tensile stress in preconditioned incisional wounds was 70% higher than controls 7 days post surgery and 50% higher than controls at day 10. Mean scores for the maximum load and tensile stress were statistically significant compared using a paired students t-test (p<0.01).

While these results describe a pulsed laser system for preconditioning tissues, some embodiments use a device that that outputs pulsed or continuous-wave laser energy for preconditioning wounds prior to the wound occurrence or postconditioning wounds after the wound occurs. Based on these results, as well as on recent evidence of laser postconditioning tissues to promote nearly scar-free wound healing that has appeared in the literature, postconditioning is effective for certain conditions. Mordon and colleagues showed accelerated and improved incisional wound healing in rats treated with 810-nm light following wound closure (Capon 2001).

Immunocytochemistry Demonstrated Activation of Hsp70.

Furthermore, a pilot clinical study conducted by the Mordon group using the 810-nm diode laser system very recently demonstrated the ability to accelerate and improve the healing process in surgical scars, with laser irradiation applied immediately after skin closure, in five patients (Capon et al., 2008). These results strongly support the laser-based approach to effectively “postcondition” existing wounds. While high levels of 810-nm light were used for these studies to initiate the conditioning effect (presumably through induction of Hsp70), this wavelength has a very large penetration depth in tissue (˜100 mm through water). Some embodiments of the present invention apply similar techniques using a device specifically optimized for laser conditioning wounds, by matching the laser wavelength to the thickness of the target tissue of interest (i.e., dermal thickness for skin).

In some embodiments, this approach of laser-induced thermal induction of the stress response is effective in enhancing wound healing when applied after a wound is already established (as opposed to pre treating tissue prior wounding) (Capon et al., 2008). While the inventors' preliminary work was done using a pulsed laser system (an Aculight Capella R-1850), a device with a tunable duty cycle with a range from about 10% up to and including about 100% (i.e., continuous wave) and uniform light output would be ideal, in some embodiments, for uniform tissue heating with a high degree of control over tissue temperature.

FIG. 7A, FIG. 7B, FIG. 7C, FIG. 7D are images and FIG. 7E and FIG. 7F are graphs that show collagen deposition and cellularity in surgical preconditioned wounds with T_{L}E_{L}. (a-b) Gomori trichrome staining after 5 days of healing (a) Control wounds reveal major disruption and only pale staining adjacent to the incisional track of non-preconditioned incisions. (b) Preconditioned wounds show normal intensely green patterns of collagen in the adjacent dermis. (c-d) Hemotoxlyin and Eosin staining after 5 days of healing (c) The surgical wound without preconditioning shows epidermal hyperplasia adjacent to the wound margin and a minimal cellular density in the granulation tissue within the incisional track. (d) A laser preconditioned surgical wound shows normal adjacent epidermis but a comparatively more robust granulation tissue within the incisional line (bar=100 μm). (e) Plot of epidermal hyperplasia over the incision and in immediate adjacent epidermis. Preconditioned surgical wounds have significantly less epidermal hyperplasia than controls, an indicator of enhanced repair (***p<0.001). (f) Plot of cell density within each wound bed per 1000 μm² (***p<0.001).

Laser Preconditioning Improves Wound Repair

FIG. 8A is a graph 801 of wound strength as a percent increase in maximum load in controls versus laser-preconditioned wounds. To compare the difference in wound healing between untreated and laser preconditioned scalpel incisions, the maximum load and tensile strength of full-thickness wound skin from mice (n=9) at days 7 and 10 after injury was measured. In FIG. 8A, the average percent (%) increase in maximum load is plotted versus day post surgery. Preconditioned incisions were 60% stronger than controls 7 days post surgery and 40% stronger than controls at day 10.

FIG. 8B is a graph 802 of wound strength as a percent increase in tensile stress in controls versus laser-preconditioned wounds. In FIG. 8B the average percent increase (%) in tensile stress is plotted versus day post surgery. Preconditioned incisional wounds were 70% (58±13%, Mean±SD) stronger than controls seven days post surgery, and 50% stronger than controls at day ten. Mean scores for the maximum load and tensile stress were statistically compared using a paired student's t-test (***p<0.01, **p<0.05, and n=9).

FIG. 8A and FIG. 8B show laser-preconditioning-protocol enhancements to wound repair. Tensiometer data for full-thickness wounds from transgenic mice (n=9) and diabetic mice (n=6) at days 7 and 10 after injury. (a) Average percent (%) increase in maximum load is plotted versus day post surgery (b) Average percent increase (%) in tensile stress is plotted versus day post surgery. The preconditioned incision wounds were ˜60% (58±13%, Mean±SD) stronger than controls 7 days post surgery, and ˜35% stronger than controls at day 10. Preconditioned surgical wounds on diabetic mice achieved only a 10% increase in wound strength at day 10. Diabetic wounds were not healed enough for tensiometry at day 7. The mean and standard deviation for the max load and tensile stress were statistically compared using a paired student's t-test (***p<0.01, **p<0.05, and n=9).

Herein the use of in vivo molecular imaging to optimize a laser-preconditioning protocol that enhances cutaneous-wound repair in a murine model is described. The laser protocol was optimized using temperature measurements and in vivo bioluminescence measurements of hsp70 expression as benchmarks. The efficacy of this optimization process was assessed using perfusion measurements, histology, immunohistochemical measurements and wound biomechanics as measurable endpoints.

Tissue preconditioning has been shown to induce tissue alterations that confer protection to subsequent damage. Thermal preconditioning appears to act, at least in part, by means of elevated heat-shock proteins. Thermal preconditioning has had a favorable impact on surgical intervention (Lepore et al., 2001; Snoeckx et al., 2001), recovery of thermally injured tissues (Baskaran et al., 2001; Merchant et al., 1998; Seppa et al., 2004), protection to ischemia reperfusion injury (Currie et al., 1988; Gowda et al., 1998; Rylander et al., 2005), and in cancer therapies (Rylander et al., 2006; Wang et al., 2004). Thermal preconditioning is reported to provide numerous effects on cells and tissues: (1) Increased resistance and survivability when exposed to subsequent lethal thermal stresses (Bowman et al., 1997); (2) Cross-protection to subsequent different stressors (i.e., mechanical stress in surgical intervention) (Parse!! and Lindquist, 1993); (3) Increased protective responses upon exposure to subsequent stresses, including increased cell migration and proliferation, reduced inflammation, and reduced apoptosis (Gabai et al., 1997; Garrido et al., 2001; Mosser et al., 1997; Samali & Cotter, 1996); and (4) Improved cutaneous-wound repair (Vigh et al., 1997).

Role of Hsp70 and Use of hsp70 as Biomarker for Preconditioning

In order to use in vivo molecular imaging as a modality to optimize a therapeutic regimen of tissue preconditioning a transgenic mouse model developed in the laboratory of Dr. Chris Contag at Stanford University was used. In the mouse model, the same cassette that we previously used was incorporated (Beckham et al., 2004; O'Connell-Rodwell et al., 2004; Wilmink et al., 2006). In this, the inducible promoter of one of the most potently inducible heat-shock proteins, hsp70A1, regulates the expression of two optically active reporter genes, luciferase and eGFP. This mouse model enables non-invasive and quantitative determination of the expression of hsp70 in a single mouse over time. We and others have previously shown that the bioluminescence signal is correlated to the amount of intracellular Hsp70 protein (Beckham et al., 2004; O'Connell-Rodwell et al., 2004; Wilmink et al., 2006), and therefore can be used as a biomarker for the induction of the hsp70 and indeed as a surrogate marker for the general activation of heat-shock response.

Clearly, preconditioning tissue by exposure to a mild heat shock does not only activate heat-shock protein (HSP) genes nor does it preferentially induce the 70 kDa HSP. In fact, numerous other genes are turned on and off in response and any number of these (both other HSP genes and other non-HSP genes) may also be responsible for the survival advantage observed. Most of these non-HSP genes function either in signal transduction or in cell growth pathways. For instance, the MAP kinase pathway plays a central role in signal transduction pathways and may contribute to the increased survivability of pretreated cells (Dinh et al., 2001). Since MAP kinases phosphorylate HSF-1, and sufficient levels of HSF-1 are required for maximal hsp70 transcription, their stimulated activity is coupled to hsp70 expression. The phosphatases DUSP1 and DUSP2 are also activated by thermal stress, and may contribute to the observed survival advantage (Ishibashi et al., 1994; Keyse & Emslie, 1992). It has been hypothesized that subsequent expression of DUSP phosphatases allow the MAP kinase pathway to “reset” thus rendering the cells responsive to subsequent stressors after an initial thermal stress (Ishibashi et al., 1994). Cell death can occur in a regulated way through apoptotic mechanisms or in an abrupt way by means of necrosis. It has been reported that severe injury causes immediate cell death, while cells that experience moderate damage take days to succumb to the insult (Rylander et al., 2005). The in vitro experiments in this report and specification demonstrated that ten-minute exposures at 44° C. induced cell proliferation, indicated by cell viability values being greater than 100%. Similar results were seen in the T_{L}E_{L} laser preconditioning protocol in vivo. Apoptosis stains also confirmed that the T_{L}E_{L} protocol caused only slight increases in apoptotic activity.

Although hsp70 expression was used as a biomarker for the preconditioning, ongoing investigations in our labs using normal and hsp70 null cells have indicated that elevated hsp70 expression has indeed a direct effect in preconditioning although some of the effect of preconditioning remains even in the absence of hsp70 (Beckham & Wilmink, 2007). Hsp70 blocks apoptosis by antagonizing apoptosis inducing factor (Ravagnan et al., 2001), preventing the recruitment of procaspase-9 (Beere et al., 2000), and by preventing the activation of stress kinases (Gabai et al., 1997). Even though the exact mechanism is not elucidated in this report and specification, the correlation between elevated hsp70 expression and reduced apoptosis is observed.

Local hyperthermia can increase perfusion to treated regions. This increase in perfusion that is observed after the T_{L}E_{L} laser preconditioning treatment may aid in the delivery of cells and growth factors to pretreated surgical sites (Song, 1984). Other studies indicate that the increased blood flow observed in preconditioned tissues may be due to increases in iNOS (Contaldo et al., 2007). Increased blow flow may increase the delivery of macrophages to wound bed, and their increased presence may correlate with improved wound debridement, increased stimulation of synthesis of collagen by fibroblasts (Heppleston and Styles, 1967), and the overall promotion of cutaneous-wound repair (Danon et al., 1989). The increased supply of various growth factors and cytokines, such as bFGF, VEGF, TGF-6 (Erdos et al., 1995; Flanders et al., 1993; Kanamori et al., 1999), may also function to enhance the repair of a preconditioned wound. It has been reported that the bFGF gene is upregulated during thermal exposures, and VEGF and TGF-6 levels are upregulated after thermal exposures (Erdos et al., 1995; Flanders et al., 1993; Kanamori et al., 1999). Since these genes are important in active wound repair they may also contribute to the survival advantage observed in laser preconditioned tissues.

Nevertheless, in correlating the laser-induced hsp70 expression in a live animal to a number of characteristics that are associated with enhanced wound healing, we have shown that this metric can be used to optimize preconditioning parameters even though the exact role of hsp70 in this process remains largely unknown.

Optimization of Preconditioning

Physical and pharmacological techniques are the main methods used to upregulate HSP expression and precondition tissues. Recently developed pharmacological agents, Bimoclomal and Geldanamycin, show clinical promise as HSP inducers or co-inducers but due to their non-specific actions and multiplicity of biochemical effects, are still considered inferior to traditional physical methods (Kiang et al., 2004; Vigh et al., 1997). Whole body hyperthermia, using a heated pad or water bath, is the classical physical preconditioning method. However, a frequent complication associated with this approach is excessive tissue dehydration (Pespeni et al., 2005). Work by Harder et al. circumvented these complications by using a heated blanket to locally induce HSP expression in restricted skin, and this technique improved skin flap survival in pigs (Harder et al., 2004). Heated blankets are attractive because they are simple and inexpensive, but they rely on the diffusion of heat and require lengthy preconditioning sessions which are not conducive to the time constraints of a clinical setting. Non-contact physical methods, such as focused ultrasound, radiofrequency, and microwave sources, also show promise since they can induce rapid and focused HSP induction in deep tissues (Madio et al., 1998; Walters et al., 1998). However, in applications targeting superficial skin, lasers are ideal since they also allow for rapid and focused induction of the heat-shock response without effecting deeper tissues (Souil et al., 2001).

In this study, a pulsed infrared (IR) diode laser was used to precondition tissues. The use of lasers for this purpose is advantageous for several reasons. First, lasers can heat tissue volumetrically and thus more uniformly, rather than depending on heat diffusion from contact to an external heating element. Second, in some embodiments, the operating parameters of the pulsed laser (A (area), Tp (laser pulse duration), H (radiant exposure or energy per pulse), repetition rate, exposure time) or continuous-wave laser (area, laser power, and exposure time) are tailored to achieve the desired spatial energy distribution and hence the depth to which tissue is heated. This allows for precise control over the spatial distribution of thermal induction of the heat-shock response and accurate dosimetry. For the work reported the output laser wavelength was fixed at 1.85 μm (1850 nm). At this wavelength the optical penetration depth in water (the main chromophore in soft tissue in this part of the spectrum) is roughly 600 μm (0.6 mm)(Hale, 1973). However, since the laser is tunable from 1.85-1.88 μm, which corresponds to a steep part of the water absorption curve, this tunability permits precise control over the depth of tissue heating. Since mouse skin is 200-300 μm thick, this laser effectively heated the entire dermis thus allowing for a relatively uniform but localized induction of hsp70 expression. Third, the laser light can be coupled into a fiber optic cable, facilitating delivery to internal tissues. Therefore, the findings reported here could be extended to minimally invasive procedures (e.g., as preconditioning for internal tissues) such as endoscopic surgery by endoscopically exposing the affected internal tissues. Taken together, these characteristics are particularly attractive and makes the described system amenable to various clinical situations, such as in the area of cardiac research (Currie et al., 1988; Gowda et al., 1998; Rylander et al., 2005).

We postulated that, in some embodiments, a successful laser-preconditioning protocol designed to enhance the repair of surgical wounds should achieve the following criteria: 1) elevate tissue temperature for prescribed exposure duration, 2) induce hsp70 levels in the tissue, 3) cause minimal irreversible tissue damage and cell death, 4) increase blood flow to the surgical site, and 5) increase wound healing strength.

Table 1 is a table of preconditioning protocols used to upregulate hsp70 expression. Many in vitro and in vivo studies have investigated hsp70-expression kinetics at various stress temperatures and exposure durations. After reviewing the literature, it appears that two general thermal regimens exist; one using high temperatures for a short duration and the other using lower temperatures for a longer exposure duration (Bowman et al. 1997; Dinh et al. 2001) summarized in Table 1.

TABLE 1

| | | | |
|---|---|---|---|
| Model: Type | Temperature (° C.) | Exposure time | Citation |

LOW TEMPERATURE LONG DURATION (T_{L}E_{L})

| | | | | |
|---|---|---|---|---|
| Animal: mouse | 41° C. | 15 | min | (Pespeni, Hodnett, et al. 2005) |
| Animal: rat | 42-42.5° C. | 15 | min | (Leger, Smith, et al. 2000) |
| Cell: rat cardiac | 42° C. | 30-60 | min | (Vigh, Literati, et al. 1997) |
| Cell: NHDF | 43° C. | 20-40 | min | (Wilmink, Opalenik et al. 2006) |
| Cell: BAEC | 41° C. | 22 | min | (Rylander, Diller et al. 2005) |
| Tissue: (RAFT) | 44° C. | 20 | min | (Wilmink, Opalenik et al. 2006) |
| Cell: NIH-3T3 | 44° C. | 40 | min | (Beckham, Mackanas et al. 2004) |

HIGH TEMPERATURE SHORT DURATION (T_{H}E_{S})

| | | | | |
|---|---|---|---|---|
| Cell: BAEC | 48° C. | 2 | min | (Rylander, Diller et al. 2005) |
| Animal: Rat | 50° C. | 3 | sec | (Souil, Capon et al. 2001) |
| Cell: NIH-3T3 | 50° C. | 35 | sec | (O'Connell-Rodwell, Shriver et al. 2004) |
| Cell: BAEC | 50° C. | 1 | min | (Rylander, Diller et al. 2005) |
| Cell: RPE | 55° C. | 3 | sec | (Dinh, Zhao et al. 2001) |
| Cell: NHEK | 55° C. | 4 | sec | (Bowman, Schuschereba et al. 1997) |
| Cell: NHEK | 58° C. | 1 | sec | (Dinh, Zhao et al. 2001) |

To a large extent, this trade off between temperature and the time to which tissue is exposed to a given temperature has long been known for tissue damage and can be formulated by the Arrhenius-rate process. The Arrhenius relationship predicts that tissue injury is linearly proportional to the time of exposure and exponentially dependent on temperature (Moritz, 1947). It assumes the basis for damage to be a biophysical process (i.e., protein denaturation) and recently was demonstrated to be useful in examining hsp70-expression kinetics (Beckham et al., 2004). While a more detailed discussion of the Arrhenius model is not provided herein, it is within the understanding of a person of skill in the art, and the underlying principle may be useful in optimizing proper combinations of temperature and time. Simply stated, as a rule of thumb for every 4° C. increase in temperature rise, the exposure time must be reduced by an order of magnitude in order to maintain the same level of damage.

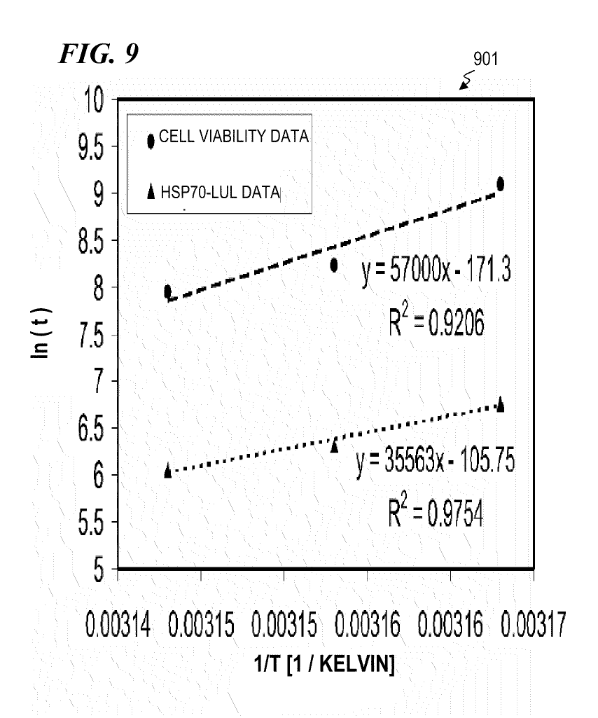

FIG. 9 is a graph 901 of an Arrhenius damage analysis. Arrhenius damage data were plotted using the equation ln(t)=Eₐ/RT−ln with ln(t)<seconds>versus 1/T<1/Kelvin>. From these plots, the slope and the y-intercept were used to calculate the activation energy Eₐ and frequency factor A.

Table 2 is a table of data used for the Arrhenius damage parameters.

TABLE 2

| Endpoint for | Frequency Factor | Activation Energy |
|---|---|---|
| Damage <Method> | (A) <s⁻¹> | (E_{A}) <J/mol> |
| Cell viability (%) | 2.4 × 10⁷⁴ | 4.7 × 10⁵ |
| Hsp70 via BLI | 8.4 × 10⁴⁵ | 3.0 × 10⁵ |

An Arrhenius analysis was conducted for our in vitro experiments and is provided in FIG. 9 and Table 2. However, preliminary studies in our laboratory suggested that there are two absolute temperatures associated with the thresholds of the induction of the heat-shock response and the induction of irreversible tissue damage. This suggested that the dosimetry for preconditioning is not as simple as a straight forward use of the Arrhenius rule of thumb. For mild temperatures (43-45° C.) a potent heat-shock response was induced but at least for the exposure times used, irreversible thermal damage (tissue whitening, blistering) is not seen even for long exposures. For higher temperatures (near 50° C.), the heat-shock response is induced but there appears to be a very fine line between inducing irreversible damage and inducing the heat-shock response and this is extremely sensitive to the exposure time. Based on this data we developed and tested in more detail, two separate laser preconditioning protocols from each thermal regimen; a low-temperature long-duration protocol (T_{L}E_{L}) and a high-temperature short-duration protocol (T_{H}E_{S}). The tissue temperatures generated by each protocol were measured in real-time using an IR camera, and proved to be critical in the optimization of each protocol. In a clinical setting, temperature measurements may not be possible, and instead temperatures could be predicted before protocol inception using thermal modeling (Rylander et al., 2006).

Maximal hsp70 expression was assessed with the transgenic hsp70A1-luc mouse. This novel mouse model provides a high throughput platform that allows us to test the effect that different laser parameters have on hsp70 expression. At both radiant exposure settings, the exposure time was varied to find the maximal hsp70 expression without inducing irreversible tissue damage or cell death. Irreversible thermal tissue damage is observed macroscopically as tissue whitening. This occurs when extracellular matrix proteins are coagulated causing visible light to be scattered and subsequently reflected, and the phenomenon is reported to occur after exposure to 65° C. for 35 seconds (Vogel & Venugopalan, 2003). In this study, a positive control lesion was generated using laser settings that whitened the mouse skin. This positive control served as a useful indicator for irreversible thermal damage and was used to compare the ensuing tissue effects in the preconditioned regions of tissues.

For the T_{L}E_{L} laser preconditioning protocol, hsp70 levels increased linearly with increasing laser exposure duration. Surprisingly, linear increases were not shown using the T_{H}E_{S} laser protocol. The data show that peak hsp70 induction of 11.65 fold is achieved with the 150-second exposure while shorter 120-second exposures only achieved four-fold induction. The large disparity in hsp70 levels between exposure durations suggested that the T_{H}E_{S} laser protocol was more difficult to tailor for specific hsp70 levels. Moreover, in the T_{H}E_{S} protocol, the temperature-time history becomes inherently difficult to control and predict since over the short exposure time the temperature-time history is dominated by the highly dynamic temperature rise and temperature decrease phases of the heating process. In summary, for the T_{L}E_{L} laser preconditioning protocol any exposure between 10-20 minutes induces sufficient hsp70 levels (about ten fold); while, for the T_{H}E_{S} protocol exposures greater than 120 seconds were required to fulfill the hsp70 requirement.

All of the exposure times tested using the T_{L}E_{L} laser preconditioning protocol exhibited sufficient hsp70 levels while inducing negligible cellular damage. In contrast, the T_{H}E_{S} protocol only had sufficient hsp70 levels using a 150-second exposure, but at this exposure duration undesired tissue damage and epidermal hyperplasia was induced. Therefore, the T_{L}E_{L} laser preconditioning protocol was determined to be superior and was selected for use in the surgical wound repair experiments. The T_{L}E_{L} laser preconditioning protocol improved the strength of wound repair in normal wounds. Preconditioned wound beds showed higher cell densities than control wounds, and this increase in cell density may confer a more concerted and robust repair process. The exact role that Hsp70 plays in preconditioning is still not entirely clear, and preconditioning may in fact be dependent or at least co-dependent on increased blood flow, increased presence of growth factors, and the reduced apoptotic activity.

In summary, in some embodiments, a method is described wherein a laser preconditioning protocol was optimized using in vivo molecular imaging and thermal infrared imaging measurements as benchmarks. Two laser protocols were investigated and described: a low-temperature long-duration protocol (T_{L}E_{L}) and a high-temperature short-duration protocol (T_{H}E_{S}). Both protocols were capable of achieving sufficient hsp70 expression levels, but the T_{H}E_{S} laser protocol required a 150-second exposure duration. This exposure duration induced significantly more epidermal hyperplasia than suitable exposures using the T_{L}E_{L} protocol, and therefore T_{L}E_{L} protocol was found to be superior for laser preconditioning, in some embodiments. The T_{L}E_{L} laser protocol induced negligible histological damage, demonstrated a positive impact on cellular proliferation while causing minimal apoptosis. The T_{L}E_{L} laser preconditioning protocol was useful in stimulating wound repair by enhancing cell migration into the wound bed, and resulted in increased wound tensile strength. We speculate that this method can be used to improve repair in a chronic wound, like those in diabetic patients. In normal conditions of wound repair, the processes that result in cutaneous healing follow a specific time course, but conditions created in diabetes impair the normal sequence of wound repair (Braddock et al., 1999). In normal wound repair, hsp70 is rapidly induced but in the chronic wound setting, hsp70 is decreased (McMurtry et al., 1999). This developed laser preconditioning protocol is useful in inducing hsp70 expression and improves wound repair. In addition, a variation to this approach may be used to treat already existing wounds rather than relying on preconditioning which are only useful for elective procedures. The methods and mouse model (hsp70A1-luc) used in this study have broader implications since they provide the framework that is amenable to the systematic design and optimization of therapeutic wound modulation and preconditioning protocols for a clinical setting.

Materials and Methods—Animal Model

Experiments were conducted in accordance with guidelines specified by the Institutional Animal Care and Use Committee (IACUC) at Vanderbilt University. Transgenic mice in which the heat-shock protein 70 (hsp70) promoter drives the luciferase and eGFP reporter genes were a generous donation from Dr. Chris Contag at Stanford University. A detailed description on the hsp70A1-luc-2A-eGFP L2G transgenic mouse has been detailed previously by Dr. Mark Mackanos (O'Connell-Rodwell et al., Submitted). Briefly, the transgenic mice are of a FVB background and contain an hsp70 cassette (FVB.hsp70A1-luc-2A-GFP). The cassette is as follows: the murine hsp70A1 promoter (Genbank accession number M76613) was attached to the luciferase coding sequence from the pGL3-Basic plasmid (Promega, Madison, Wis.) as described previously and fused, in frame, to the ORF of the enhanced green fluorescent protein (eGFP; Clontech, Palo Alto, Calif. U.S.A.) with 54 base pairs (bp) of the FMDV 2A sequence followed by 24 by of polylinker (O'Connell-Rodwell et al., 2004). The cDNA for (eGFP) and luciferase (luc) vector are located downstream from the hsp 70 promoter. Therefore, whenever the transcriptional factors are present which induce hsp70 mRNA transcription, these bicistronic reporters are transcribed and translated. Resultantly, the GFP and luciferase gene products emit light which can be used as a surrogate marker for hsp70 gene activity levels, as previously shown (Wilmink et al., 2006).

In Vitro Cell Culture Experiments

Dermal fibroblasts were isolated from the hsp70A1-luc-EGFP mouse and immortalized with the hTERT gene for in vitro experiments. Two day old hsp70A1-luc-eGFP mice were euthanized and their dermis was excised and digested in 0.2% collagenase (type III) in DMEM containing 10% fetal bovine serum at 37° C. overnight in culture incubator. Cells were subsequently centrifuged at 1000×g for 5 minutes, plated in DMEM (10% fetal bovine serum and pen/strep) and incubated overnight. Media was replaced after 72 hrs, and the cells were replenished with media every 2-3 days until confluent.

On day 1, immortalized mouse dermal fibroblasts (MDF) were plated in six well (9.62 cm²) tissue culture plates with 10⁵ cells per well in 2 ml of culture medium (Costar, Fisher Scientific, Sewanee, Ga. U.S.A.). On day 3 the culture plates were sealed with parafilm and then heat shocked by floating them in a water bath at 43, 44, or 45° C. for varying exposure times (0 to 120 min). Heat-shocked cells were placed back in a 37° C., 5% CO₂ incubator (Forma Scientific) for defined time periods before analysis.

Bioluminescent Imaging and Cell Viability Assays for Mouse Dermal Fibroblasts

Cells (control and heat shocked) were imaged for bioluminescent intensity as previously described (Wilmink et al., 2006). In brief, D-luciferin potassium salt (Biosynth AG, Switzerland) is diluted in ddH₂O to a concentration of 2.25 mg/ml. Before each imaging session, 150 IJL of substrate was added to each well. After delivery of the substrate, the cells were incubated for five minutes before each imaging session to ensure for maximal bioavailability of substrate. Luciferase-induced bioluminescent light emission was measured 12 h after heat using an IVIS 100 bioluminescent imaging system (Xenogen, Alameda, Calif. U.S.A.) and light emission was quantified using Living Image analysis software (v2.12, Xenogen). Light emission was measured from each well and was quantified as a photon flux in units of total number of photons emitted/second.

In order to provide information to supplement the BLI data, cell viability assays using sensitive fluorometric methods were performed. Forty-eight hours after thermal stress, 300 pl of CellTiter-Blue Cell Viability Assay was added to each well and incubated for 2 hours (G808B, protocol, TB317, Promega, Madison, Wis., U.S.A.) and analyzed using a Bio-Tek Synergy HT fluorescent plate reader. Fluorescent values for each sample were normalized to controls and expressed as a percent viability.

Laser-Preconditioning Experiments In Vivo

Two days before laser experiments, mice were anesthetized with isoflurane in an isoflurane vaporizer (V-10 Series, VetEquip Inc, Pleasanton, Calif., U.S.A.) and a rectangular area of the dorsal fur was removed with a clipper. The remaining hair remnants above the surface were removed using depilatory cream and the skin was thoroughly cleansed with water to remove residual cream. The mice were then returned to animal care for 2 days.

Tissue preconditioning was accomplished with an Aculight Renoir diode laser (Renoir product available from Aculight, Bothel, Wash., U.S.A.). Laser light was coupled into a 600-μm-diameter multimode silica fiber for transmission and delivery. The bare fiber tip was positioned 2.5 cm above the dorsal skin of the mouse.

Two Laser-Preconditioning Protocols

The Arrhenius-rate process that describes thermal damage suggests that tissue preconditioning can be achieved either by using short exposures (seconds) at high temperatures (50-60° C.) or by using longer exposures (minutes) at lower temperatures (40-50° C.) (Bowman et al., 1997; Dinh et al., 2001). In the literature, various thermal protocols have been used to maximally induce hsp70 expression, as shown in Table 1. We elected to investigate laser preconditioning protocols using two differing thermal regimens. One experimental condition used a low-temperature long-duration protocol (T_{L}E_{L}) and the second experimental condition tested a high-temperature short-duration protocol (T_{H}E_{S}). The laser parameters used for the T_{H}E_{S} protocol were λ=1.85 μm (wavelength=1850 nm), repetition rate 50 Hz, Tp=2 ms (pulse duration=0.002 seconds), beam diameter 5 mm, radiant exposure per pulse or H=9.17 mJ/cm², and total exposure duration=30-150 seconds. The optimized preconditioning T_{L}E_{L} parameters: X=1.85 μm, repetition rate 50 Hz, Tp=2 ms, beam diameter 5 mm, radiant exposures per pulse or H=7.64 mJ/cm², total exposure duration=10-40 minutes. The laser parameters used for a positive control were λ=1.85 μm, repetition rate 50 Hz, Tp=2 ms, beam diameter 5 mm, H=30 mJ/cm², total exposure duration=60 seconds (or 1.5 W/cm²). The positive control induced tissue whitening and was used as an indicator of irreversible tissue damage.

Optimizing Laser-Preconditioning Protocols

Laser-preconditioning protocols (temperature and exposure time) were optimized using thermal infrared, bioluminescent, and laser perfusion Doppler imaging methods. Tissue temperatures were measured in real time during laser preconditioning treatments using an infrared (IR) camera (A20 series, FLIR Systems, Portland, Oreg., U.S.A.). ThermaCAM researcher 2.8 SR-3 software was used to analyze the data. The camera is sensitive to temperature changes±0.1° C.

Bioluminescent Imaging of Living Mice

After mice were preconditioned with the laser, the hsp70-induced luciferase bioluminescence was measured at various time points following heat shock using an IVIS 200 BLI system (available from Xenogen, Alameda, Calif.). Fifteen minutes before each imaging session the mice were anesthetized with isoflurane and injected (27-gauge syringe) with 15 mg/ml of luciferin substrate intra-peritoneally (i.p.) using a dose of 10 pl/gram body weight. Mice were imaged at 0, 3, 6, 9, 12, 15, and 24 hours after the start of the laser treatment. Mice were placed in the imaging chamber on a 37° C. stage, and bioluminescent images were acquired using an integration time of 2 minutes. Bioluminescent data were represented with a false color scheme representing the regions of varying light emission, and quantified using LivingImage analysis software (v2.12, Xenogen). Light emissions from specified regions of interest (ROIs) were quantified as a photon flux in units of total number of photons emitted/second/R01 (p/s/ROI). To account for mouse-to-mouse variability, measured BLI values for each wound were normalized to the BLI value of the unwounded but shaved dorsum. This normalization was conducted at each time point. The normalization procedure yielded a fold induction number which is indicative of the relative magnitude of hsp70 expression in each wound compared to normal, untreated tissue.

In order to ensure each transgenic mouse exhibited comparable sensitivity to heat exposure, the laser was used on each mouse to induce a positive control lesion using radiant exposure H=30 mJ/cm² for 60 seconds, corresponding to a peak temperature of 65° C. This lesion was positioned 0.5 cm anterior to the tail on the dorsum of each mouse. Animals that showed greater than 20% deviation from the mean in response to this control tissue were excluded from the studies.

Laser Doppler Perfusion Imaging

Dynamic blood flow on the mice dorsum was mapped with a high resolution PIM-2 Laser Doppler perfusion imager (Perimed Inc., North Royalton, Ohio U.S.A.). The parameter settings during the measurement were: scanning area, 20×20 mm; high resolution scanning; distance between the scanner head and wound, 17.8 cm. The measurement of the image and perfusion value was carried out by the LDIwin2.6 software package (Perimed AB, Sweden). The extent of blood perfusion for each sample was normalized to an untreated control region. Normalized perfusion values were collected at 10 minutes, 3 days, and 10 day intervals after laser treatment.

Laser Preconditioning to Improve Wound Repair

Twelve hours after laser preconditioning, four full thickness longitudinal incisions, separated by 1.5 cm and each 1.0 cm in length, were made on the dorsum of each mouse. The four lesions consisted of 2 laser preconditioned areas and 2 untreated control areas. The anterior preconditioned wounds were compared to anterior control regions of tissue, and vice versa. This was done to reduce the bias due to the anatomical position of each wound. Wounds were closed with 7.5-mm clips, and the clips were removed on day five. Immediately after wounding with the surgical scalpel, bioluminescence images of the mice were taken. The BLI images served to verify that the laser-preconditioning-protocol-induced hsp70 expression at the surgical wound site.

Tensile Strength Measurement

To compare the difference in wound healing between scalpel incisions (control) and laser preconditioned scalpel incisions, maximum loads and tensile strengths of full-thickness wound skin from transgenic mice (n=9) were measured at days 7, 10, and 14 after injury. Tensiometry was performed on skin incisions using the Instron 5542 tensiometer (Instron, Canton, Mass., U.S.A.) within 3 hours after tissue harvest (Benn et al., 1996). All tensiometry was performed in a blinded fashion.

Histologic Parameters of Wound Healing

In order to evaluate cellular structure, damage, and collagen deposition a subset of the test animals were euthanized to provide mouse skin for histological and immunohistochemical analysis. Skin samples were collected 12, 48, 72, 120, and 240 hours after wounding. Standard histological processing of formalin-fixed, paraffin-embedded samples included H&E and Gomori's trichrome as previously described (Wilmink et al., 2007; Wilmink et al., 2006). An Olympus Vanox-T AHZ microscope equipped with a Pixera Pro 600 ES camera was used to image the slides. Olympus Plan Opo Primary Objectives (10×, and 20×) were used. The depth of damage was measured using Image Pro Plus software.

Immunohistochemistry

In order to evaluate the effects of laser preconditioning protocols have on wound repair, an immunohistochemical study was conducted on tissue sections. Paraffin sections (7 μm) of mouse back skin were analyzed by immunological methods. Macrophages expressing the F4/80 surface marker were immunostained with a rat monoclonal antisera. Cell Proliferation was evaluated using a Ki-67 antigen. Apoptotic cells were selectively highlighted within tissue samples by immunostaining for cleaved caspase-3.

Apoptotic cells with fragmented DNA were visualized with DeadEnd Colorimetric TUNEL System. Immunohistochemistry was performed using the following antibodies: F4/80 (CI:A3-1, F4/80; Serotec, Inc., Raleigh, N.C., U.S.A.), Ki67 (VP-RM04, Vector Laboratories, Burlingame, Calif.), DeadEnd Calorimetric TUNEL System (G3250, Promega Corporation, Madison, Wis., U.S.A.), and Caspase-3 (9PIG748, Promega, Madison, Wis., U.S.A.). The Dako Envision+HRP/DAB+System (DakoCytomation) was used to produce localized, visible staining. The slides were lightly counterstained with Mayer's hematoxylin, dehydrated and coverslipped. Olympus Vanox-T AHZ microscope (Olympus America, Center Valley, Pa., U.S.A.), Olympus Plan Opo Primary Objectives (10×, and 20×), and Image Pro Plus software (Media Cybernetics, Bethesda, Md., U.S.A.) were used to image the slides.

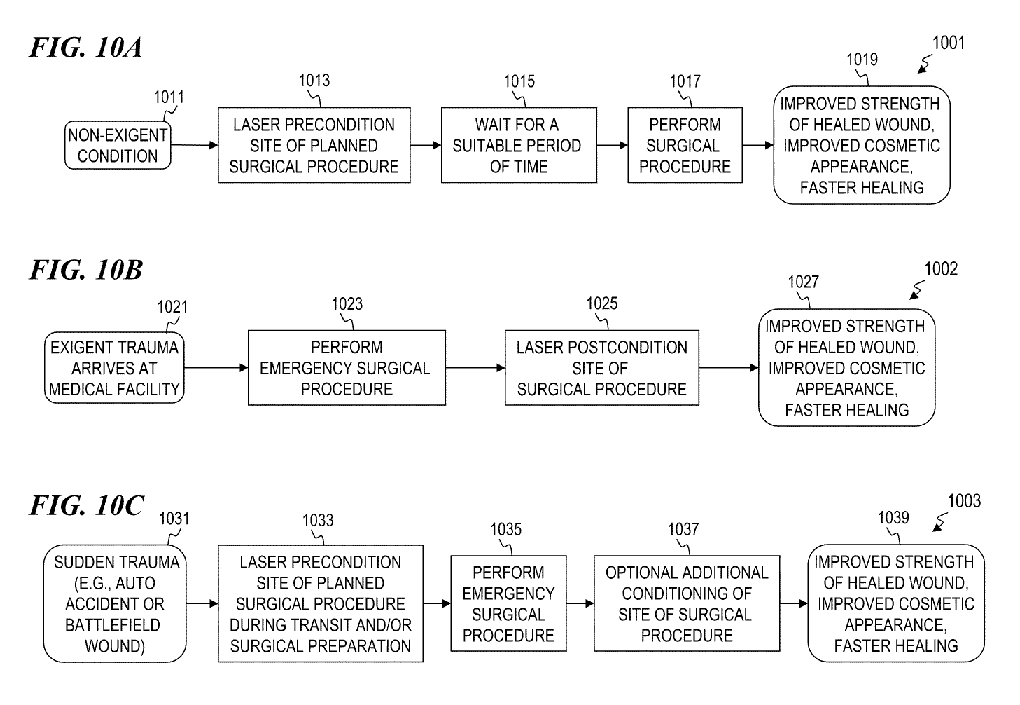

FIG. 10A is a block diagram of a high-level laser-preconditioning method 1001 according to some embodiments of the present invention. In some embodiments, method 1001 includes preconditioning the site of a planned surgery for treating a non-exigent condition 1011 (such as an elective surgery other than an exigent trauma that should be surgically treated immediately). In some embodiments, at block 1013, the site of the planned surgery (e.g., the incision site and some surrounding skin, in some embodiments) is preconditioned by exposing the tissue to laser energy according to a protocol such as, for example, the T_{L}E_{L} protocol (low fluence, long exposure) described above generating tissue temperatures between 43 and 44° C. for a predetermined exposure duration of about ten to thirty minutes (e.g., 20 minutes). In some embodiments, the combination of block 1013 and block 1015 results in elevated tissue temperature for prescribed exposure duration, increased Hsp70 protein levels induced in the tissue, minimal irreversible tissue damage and cell death, and increased blood flow to the surgical site. In some embodiments, at block 1015, the method includes waiting for a predetermined period of time of about eight to twelve hours (e.g., 12 hours) or about twenty-four hours (since, in some embodiments, the hsp70 response is biphasic with a first maximum at about eight to twelve hours and a second maximum at about twenty-four hours). At block 1017, the surgical procedure is performed. At block 1019, the improved result (e.g., improved wound-healing strength, improved cosmetic result, and the like) is observed.

FIG. 10B is a block diagram of a high-level laser-preconditioning method 1002 according to some embodiments of the present invention. In some embodiments, method 1002 includes treating an exigent condition 1021 (such as an emergency surgery for an exigent trauma that should be surgically treated immediately). At block 1023, at least the exigent portion of the surgical procedure is performed. In some embodiments, at block 1025, the site of the already-performed surgery (e.g., the incision site and some surrounding skin, in some embodiments) is postconditioned by exposing the tissue to laser energy according to a protocol such as, for example, the T_{L}E_{L} protocol (low fluence, long exposure) described above generating tissue temperatures between 43 and 44° C. for a predetermined exposure duration of about ten to thirty minutes (e.g., 20 minutes). In some embodiments, the block 1025 results in elevated tissue temperature for prescribed exposure duration, increased Hsp70 protein levels induced in the tissue, minimal irreversible tissue damage and cell death, and increased blood flow to the surgical site. At block 1027, the improved result (e.g., improved wound-healing strength, improved cosmetic result, and the like) is observed.

FIG. 10C is a block diagram of a high-level laser-preconditioning method 1003 according to some embodiments of the present invention. In some embodiments, method 1003 includes treating an exigent condition 1031 (such as an emergency surgery for an exigent trauma that should be surgically treated immediately), wherein the patient must be transported from the geographical location where the injury took place (such as an automobile accident or a battlefield casualty) to the hospital or MASH unit where the surgery will be performed. For those situations where the transportation may take the ten-or-so minutes that the HSP-conditioning protocol of the present invention calls for, the conditioning does not add any time for the other surgical functions that will take place. In some embodiments, at block 1033, the site of the wound and planned surgery (e.g., the incision site and some surrounding skin, in some embodiments) is preconditioned by exposing the tissue to laser energy according to a protocol such as, for example, the T_{L}E_{L} protocol (low fluence, long exposure) described above generating tissue temperatures between 43 and 44° C. for a predetermined exposure duration of about ten to thirty minutes (e.g., 20 minutes), as limited of course by the exigent treatment of the patient during transit to the surgical facility. At block 1035, the surgical procedure is performed. In some embodiments an optional procedure is used, wherein at block 1037, the site of the already-performed surgery (e.g., the incision site and some surrounding skin, in some embodiments) is postconditioned by exposing the tissue to laser energy according to a protocol such as, for example, the T_{L}E_{L} protocol (low fluence, long exposure) described above generating tissue temperatures between 43 and 44° C. for a predetermined exposure duration equal to that portion of the about ten to thirty minutes (e.g., 20 minutes) that could not performed during the transit described in block 1033. At block 1039, the improved result (e.g., improved wound-healing strength, improved cosmetic result, and the like) is observed.

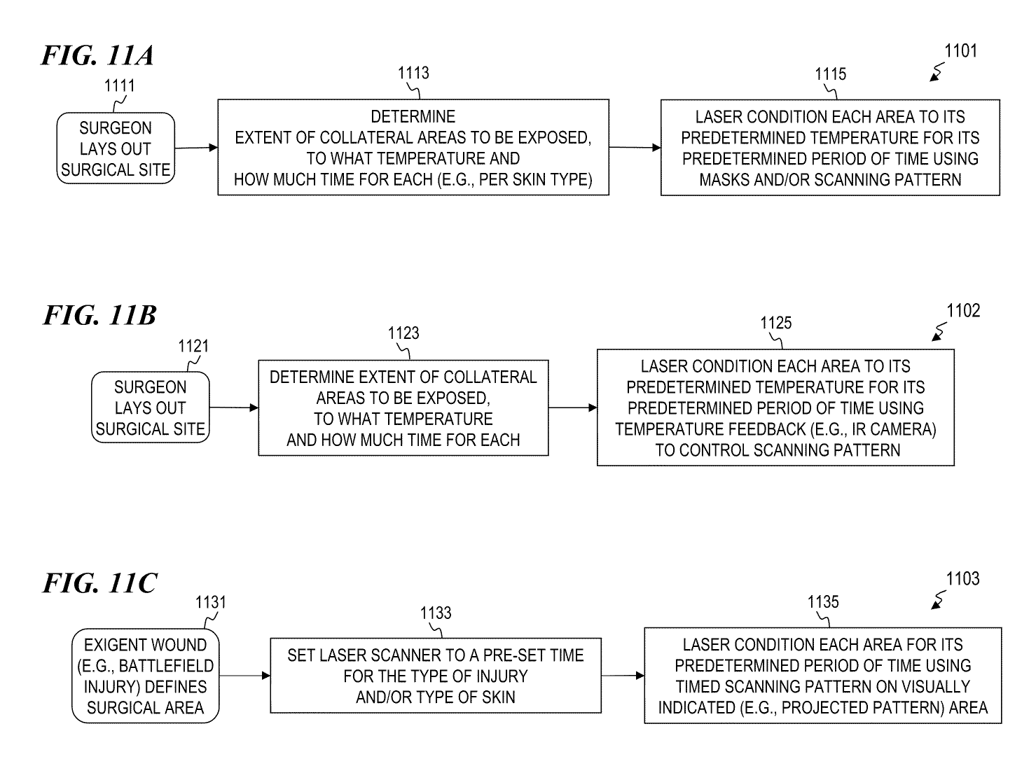

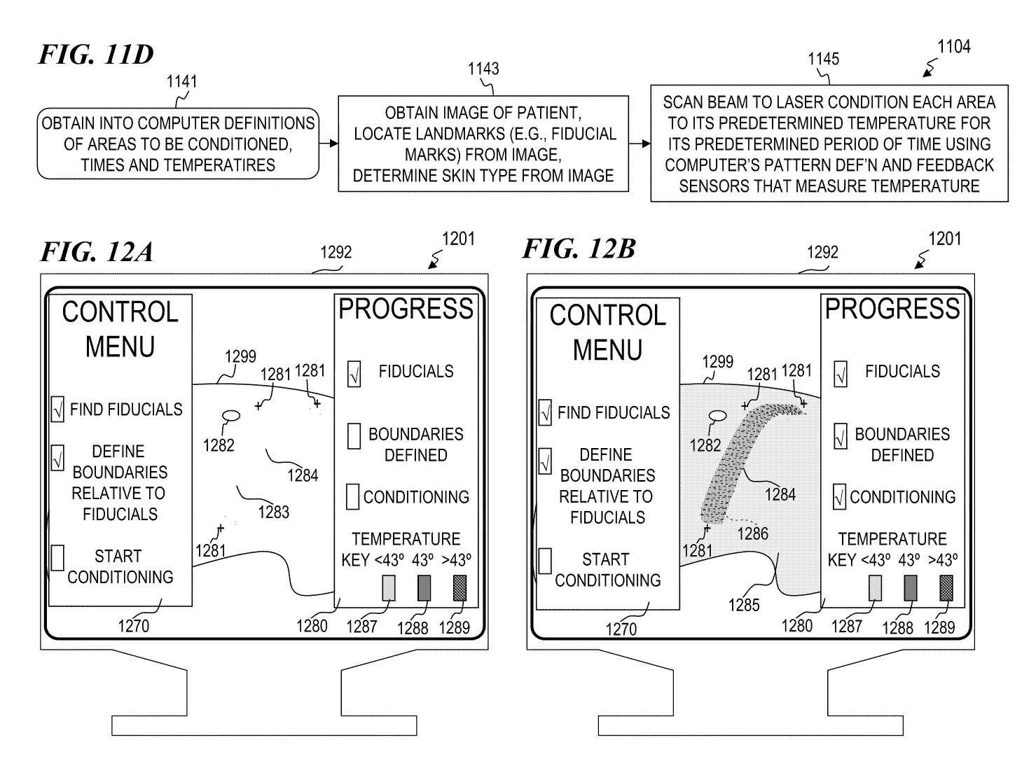

FIG. 11A is a block diagram of a more detailed laser-preconditioning method 1101 according to some embodiments of the present invention. In some embodiments, method 1101 is used for the blocks 1013, 1025, 1033 and/or 1037 that form portions of the above-described methods of FIG. 10A, 10B or 10C. At block 1111, the surgeon or other medical practitioner lays out the surgical site (e.g., (a) drawing lines or fiducial marks on the skin of the patient where the incision(s) will be made or outlining an area within which the incision(s) will be made, (b) applying a masking fabric, sticky tape, insulating material and/or other material around the planned surgical site that provides a stencil limiting the lateral extent of or defining areas where laser energy is applied for laser preconditioning or postconditioning of the planned surgical site, and/or (c) defining lines or areas on a computer-screen image of a relevant portion of the patient, where the computer-drawn lines are used as data to help the computer control where laser energy will be directed during the conditioning procedure, whether preconditioning or postconditioning is used). At block 1113, the surgeon or other medical practitioner determines the appropriate temperature and time required for laser conditioning of the planned surgical site. In some embodiments, the appropriate temperature and time for laser conditioning is determined by the type of surgery being performed (e.g., breast augmentation, cesarean section, coronary artery bypass surgery or the like) which necessarily establishes the type of skin or tissue being conditioned and the desired depth of the conditioning (e.g., an incision on the back of a patient's hand would require a shallow depth of laser conditioning, whereas an incision on a patient's stomach which has thicker tissue would require the depth of laser conditioning extend deeper into the tissue). At block 1115, the surgeon or other medical practitioner performs the laser preconditioning or postconditioning by applying laser energy to the planned surgical site for the predetermined period of time in order to achieve the desired temperature as predetermined at block 1113. In some embodiments, the surgeon or other medical practitioner inputs the predetermined temperature and period of time into a laser controller that is used to control the conditioning laser's output energy and therefore the conditioning temperature and period of time.

FIG. 11B is a block diagram of a more detailed laser-preconditioning method 1102 according to some embodiments of the present invention. In some embodiments, method 1102 is used for the parts 1013, 1025, 1033 and/or 1037 of the above-described methods of FIG. 10A, 10B or 10C. At block 1121, the surgeon or other medical practitioner lays out the surgical site (e.g., (a) drawing lines or fiducial marks on the skin of the patient where the incision(s) will be made or outlining an area within which the incision(s) will be made, (b) applying a masking fabric, sticky tape, insulating material and/or other material around the planned surgical site that provides a stencil limiting the lateral extent of or defining areas where laser energy is applied for laser preconditioning or postconditioning of the planned surgical site, and/or (c) defining lines or areas on a computer-screen image of a relevant portion of the patient, where the computer-drawn lines are used as data to help the computer control where laser energy will be directed during the conditioning procedure, whether preconditioning or postconditioning is used). At block 1123, the surgeon or other medical practitioner determines the appropriate temperature and time required for laser conditioning of the planned surgical site. In some embodiments, the appropriate temperature and time for laser conditioning is determined by the type of surgery being performed (e.g., breast augmentation, cesarean section, coronary artery bypass surgery, skin cancer removal or the like) which necessarily establishes the type of skin or tissue being conditioned and the desired depth of the conditioning (e.g., an incision on the back of a patient's hand would require a shallow depth of laser conditioning, whereas an incision on a patient's stomach which has thicker tissue would require the depth of laser conditioning extend deeper into the tissue). At block 1125, the surgeon or other medical practitioner performs the laser preconditioning or postconditioning by applying laser energy to the planned surgical site for the predetermined period of time in order to achieve the desired temperature as predetermined at block 1123. In some embodiments, the surgeon or other medical practitioner inputs the predetermined temperature and period of time into a laser controller having a temperature feedback mechanism (e.g., in some embodiments, an IR camera is used to measure the temperature of the tissue receiving the conditioning laser energy to determine when the desired therapeutic temperature has been reached and to prevent the temperature from raising past the regime wherein therapeutic conditioning is achieved to the regime wherein tissue damage is generated) that is used to control the conditioning laser's output energy and therefore the conditioning temperature and period of time.

FIG. 11C is a block diagram of a more detailed laser-preconditioning method 1103 according to some embodiments of the present invention. In some embodiments, method 1103 is used for the parts 1013, 1025, 1033 and/or 1037 of the above-described methods of FIG. 10A, 10B or 10C. At block 1131, a patient with an exigent wound (e.g., a battlefield wound, an automobile accident, a gunshot wound or the like) is presented to the surgeon or other medical practitioner and the extent of the exigent wound defines the region of tissue to be conditioned by the conditioning laser. At block 1133, the surgeon or other medical practitioner determines the appropriate temperature and time required for laser conditioning of the exigent wound. In some embodiments, the appropriate temperature and time for laser conditioning is determined by the type of wound being treated (e.g., gunshot wound, superficial cuts, shrapnel wound or the like) and the location of the wound which establishes the type of skin or tissue being conditioned and the desired depth of the conditioning (e.g., a wound on the back of a patient's hand would require a shallow depth of laser conditioning, whereas a gunshot wound in a patient's stomach which has thicker tissue would require the depth of laser conditioning extend deeper into the tissue). At block 1135, the surgeon or other medical practitioner performs the laser postconditioning by applying laser energy to the exigent wound for the predetermined period of time in order to achieve the desired temperature as predetermined at block 1133. In some embodiments, the surgeon or other medical practitioner inputs the predetermined temperature and period of time into a laser controller that is used to control the conditioning laser's output energy and therefore the conditioning temperature and period of time. In some embodiments, a timed scanning pattern is used during the application of the laser conditioning energy to achieve the desired temperature and period of time.

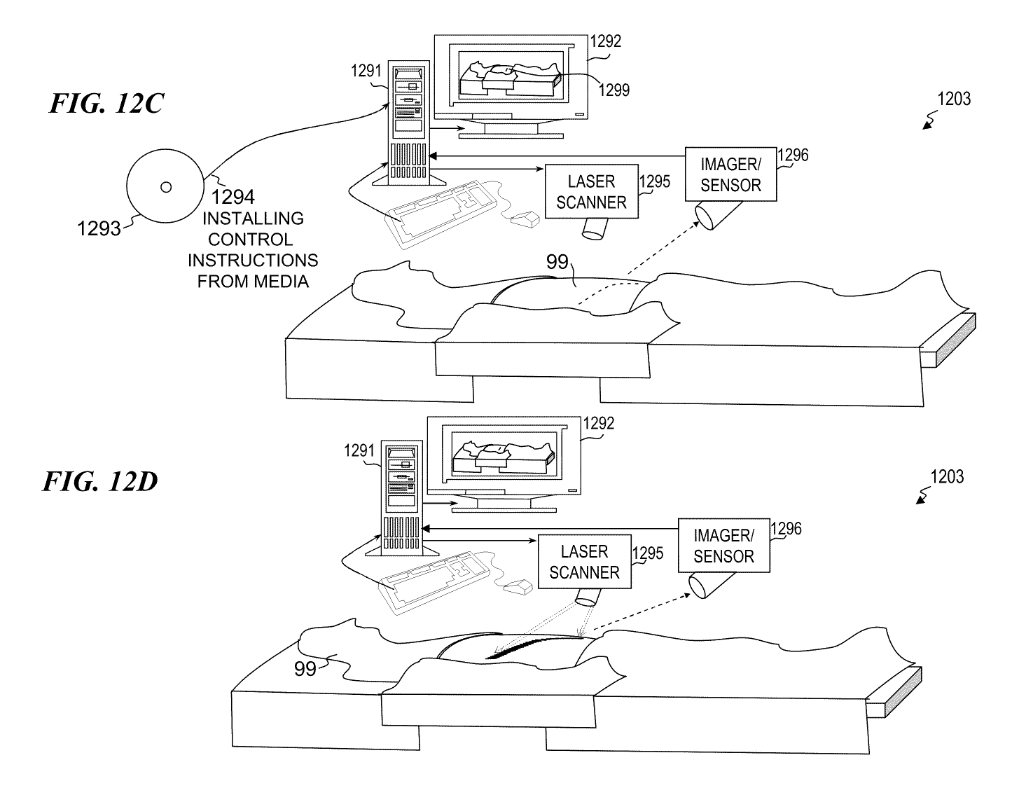

FIG. 11D is a block diagram of a more detailed laser-preconditioning method 1104. In some embodiments, method 1104 is used for the parts 1013, 1025, 1033 and/or 1037 of the above-described methods of FIG. 10A, 10B or 10C. At block 1141, a patient is presented to the surgeon or other medical practitioner who determines the extent of the region of tissue to be conditioned by the conditioning laser, and inputs the defined area into a computer (e.g., by drawing a centerline (from which a suitable lateral extent will be conditioned) or a boundary (within which the tissue will be conditioned) onto an image of the patient on a computer display (see FIG. 12A)). At block 1141, the surgeon or other medical practitioner also optionally uses the computer to help determine the appropriate temperature and time required for laser conditioning of the exigent wound. In some embodiments, the appropriate temperature and time for laser conditioning is determined by the type of wound being treated (e.g., gunshot wound, superficial cuts, shrapnel wound or the like) and the location of the wound which establishes the type of skin or tissue being conditioned and the desired depth of the conditioning (e.g., a wound on the back of a patient's hand would require a shallow depth of laser conditioning, whereas a gunshot wound in a patient's stomach which has thicker tissue would require the depth of laser conditioning extend deeper into the tissue). At block 1143 (which, in some embodiments, is performed before some or all of the functions described for block 1141), an image of the patient is obtained, and computer software (e.g., machine-vision software) is used to locate landmarks on the image (e.g., physical characteristics of the patient or fiducial marks made on the patient by the physician). Optionally, the patient's skin type (e.g., density of melanin, and like characteristics that affect laser absorption and/or scattering) is obtained from the image. At block 1145, the device, under the supervision of the surgeon or other medical practitioner, performs the laser preconditioning and/or postconditioning by applying laser energy to the designated area and volume of tissue for the predetermined period of time in order to achieve the desired temperature as predetermined at block 1141.

FIG. 12A is a block diagram of a controller computer display 1201. In some embodiments, display monitor 1292 is used to display graphical and/or textual information. In some embodiments, the display includes a control menu 1270 used to control the overall and/or detailed operation of the device. In some embodiments, this includes eliciting and receiving user input (e.g., using a graphical user interface that includes a mouse, touch-screen-input or other device) that allows the user to define the area to be treated (e.g., by marking on an image 1299 of the patient, wherein a body feature 1282 (such as a navel, crease or nipple) and/or fiducial marks 1281 on the patient are located, a defined incision location 1283 and/or the boundary 1284 are manually input and/or determined by (or with the assistance of) machine-vision software. For example, a menu having check boxes that command the device to perform certain functions (such as locating fiducial mark(s), defining boundaries relative to the fiducial marks, and starting one or a plurality of selectable conditioning protocols). In some embodiments, another area 1280 of the screen is used to indicate the current operational mode and/or the progress of the procedure (e.g., having a plurality of checkpoints such as having located the fiducial marks, having defined the boundaries relative to the fiducial marks, and having started the one or a plurality of selectable conditioning protocols) and or showing a key that indicates what temperatures have been achieved for the various areas on the image of the patient.

FIG. 12B is a block diagram of a controller computer display 1201 at another point in time, wherein here the temperature (e.g., as determined by a thermal imaging device) of the patient both inside and outside the defined area 1284 of the conditioning is shown by various colors and/or patterns on the image. In some embodiments, the scanning patter 1286 is also indicated. In some such embodiments, the scanning pattern shows where additional energy is being projected (e.g., because the tissue's temperature is below that which is desired) and areas where no additional energy is going (e.g., because the tissue's temperature is at or above that temperature which is desired).

FIG. 12C is a block diagram of a controller system 1203 according to some embodiments. In some embodiments, a computer-readable storage medium 1293 is operatively coupled to a suitable information processing device 1291 (e.g., a programmable computer), wherein the medium includes instructions and/or control-data structures to cause information processing device 1291 to execute one of the methods described herein. In some embodiments, information processing device 1291 controls a laser scanner 1295 and receives image information from imager/sensor 1296. In some embodiments, sensor 1296 includes a thermal imager configured to determine tissue temperature for a plurality of areas on patient 99, and to send signals that can be used to display an image 1299 of patient 99 that shows temperatures in substantially real time as the procedure is performed. In some embodiments, sensor 1296 also obtains image information usable by machine-vision software in information processing device 1291 to detect the ficucials and other image information usable to control the scanning function. In some embodiments, the dotted line on patient 99 (e.g., such as dotted line 1283 on FIG. 12A) represents fiducial marks placed on the patient by the doctor that define where a planned operation is to take place, which help define where the conditioning will take place.

FIG. 12D is a block diagram of a controller system 1203 at another point in time according to some embodiments of the present invention. In some embodiments, the zigzag line on patient 99 (e.g., such as zigzag scanning line 1286 on FIG. 12B) represents the scanning pattern on the patient showing where the conditioning scanning is taking place, and, in some embodiments, is shown by scanning a visible-light laser pattern in the same locations and pattern as the IR conditioning laser beam is scanned, which helps show the doctor where the conditioning is taking place. In the embodiment shown in FIGS. 12A-12D, a whole-body-capable device 1203 and its operation are shown, however, in other embodiments, smaller devices suitable for treatment of a signal extremity or small area of the body are used. In still other embodiments, an endoscopic delivery device and imaging device are used, with a corresponding control information processing device 1291 and display are used.

In some embodiments, the pre-existing wound site is laser treated to remove ragged edges. In some embodiments, this is used for so called laser-debridement applications—including but not limited to burn-wound management (chemical and/or thermal burns), ulcers and chronic-wound applications.

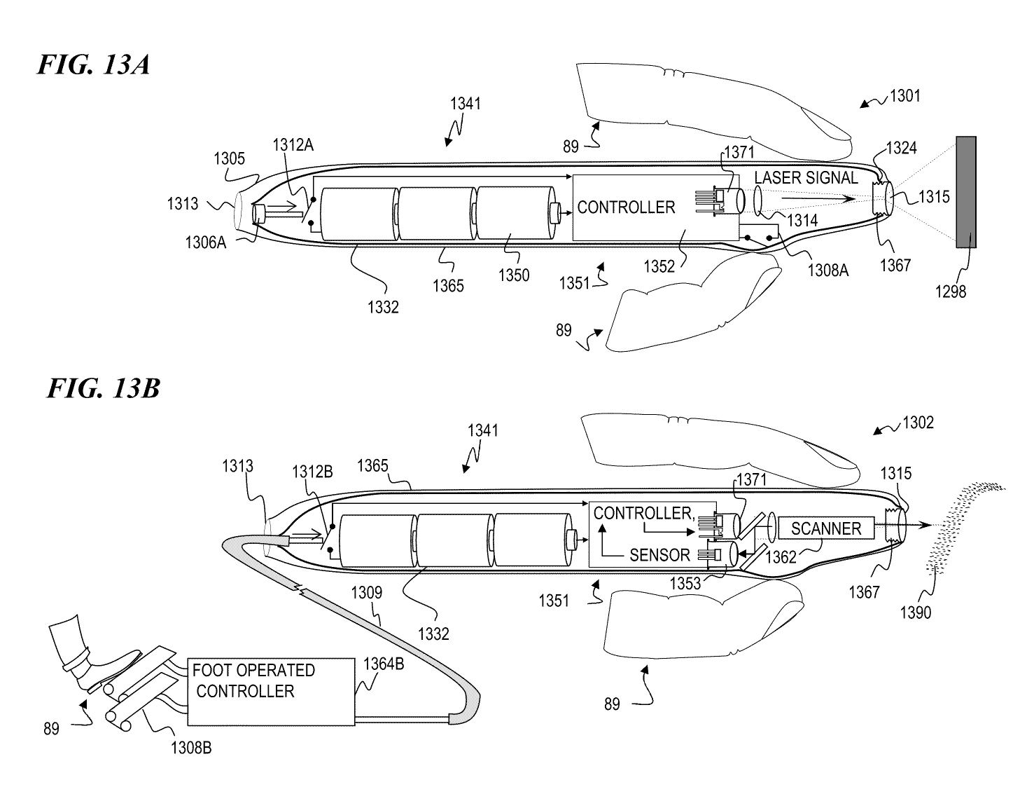

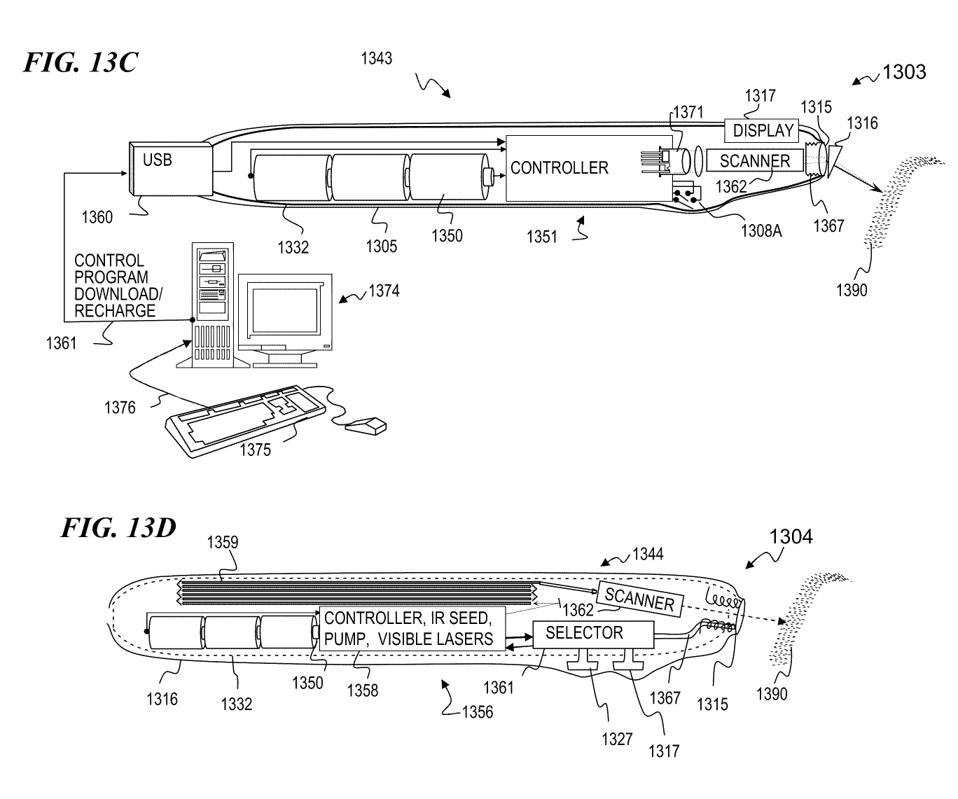

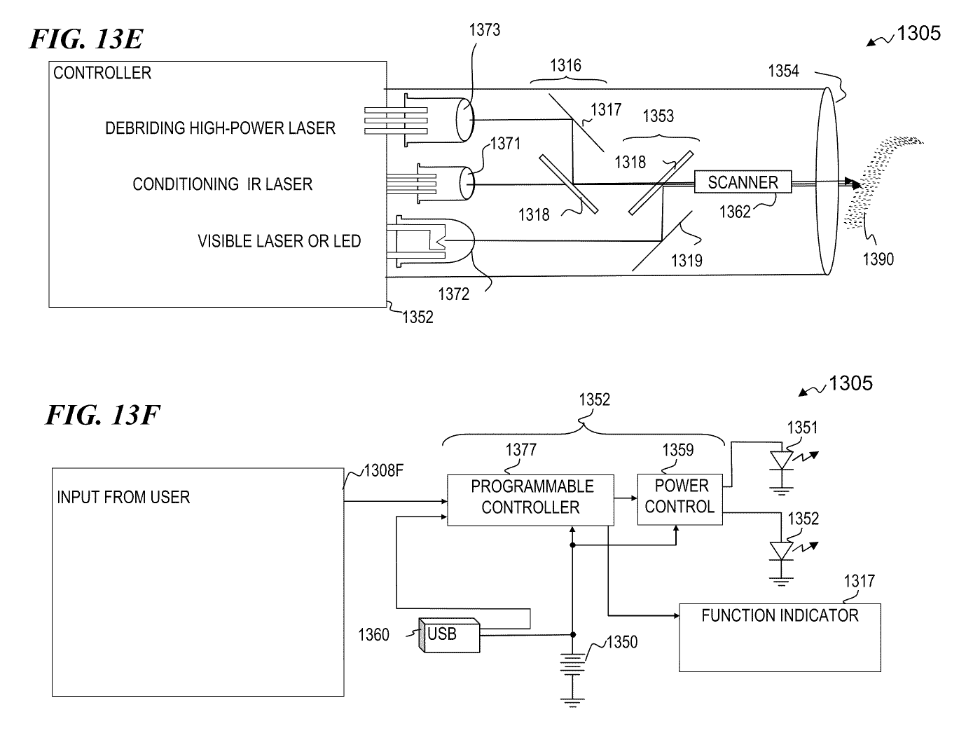

FIG. 13A is a block diagram of a battery-operated tissue-conditioning laser handpiece system 1301. In some embodiments, system 1301 uses a battery-operated tissue-conditioning handpiece 1341. In other embodiments (not shown here), a power cord delivers power for the controller and/or light emitting devices from an external power supply. In some embodiments, handpiece 1341 is similar to that described in co-pending U.S. patent application Ser. No. 11/536,639, which is incorporated herein by reference, except that the output beam is configured to heat tissue to a controlled temperature rather than to stimulate nerves. In some embodiments, tissue-conditioning handpiece 1341 outputs a pattern of tissue-heating light to condition tissue for improved healing response and visible-indicating light that shows where the tissue-heating light is being projected. In some embodiments, the tissue-heating light radiation has a power level (e.g., in some embodiments, one to five watts) and a tunable wavelength (e.g., in some embodiments, IR light having a wavelength of between 1840 and 1870 nm, while, in other embodiments, light of any suitable wavelength including visible and ultraviolet, that provide the desired penetration depth and/or heating profile to enhance HSP production without excessive protein denaturing) suitable for conditioning tissue to produce enhanced levels of Hsp70 over a suitable area and to a suitable depth while being relatively eye-safe for the operator and patient. In some embodiments, a laser-diode assembly 1371 is operatively coupled to project a pattern of light via a tip 1367 that contains one or more light-transmitting optics, optical gratings or lenses 1315. Some embodiments further include one or more lenses 1314 for beam focusing, collimating, and/or shaping. In some embodiments, control of this IR and/or visible light is via a finger trigger 1308A (which, in some embodiments, includes one or more internal separately activatable switches) being pressed or otherwise activated by user 89—typically by the finger or thumb of user 89. In some embodiments, trigger 1308A includes a flexible membrane portion of housing 1332 that covers an internal switch that is operatively coupled to a laser controller/power controller 1352 (alternatively designated as light-emitting-source controller 1352) that together with laser/light-emitting-source assembly 1371 form light source 1351. In some embodiments, power source 1350 (also called self-contained energy source 1350, which includes, e.g., batteries, in some embodiments) is interruptably connected to light source 1351 via on-off switch 1312A. In some embodiments, tip 1367, on-off switch 1312A and handpiece housing or handle 1332 are grouped together and protected via a disposable replaceable sheath 1365 to form handpiece 1341. In some embodiments, tip 1367 forms a part of handpiece housing 1332, and these are together inserted into the disposable sheath 1365 via an opening 1313, which is then folded over and sealed (e.g., via pressure-sensitive adhesive). In other embodiments, tip 1367 and its lens 1315 form a part of the disposable sheath 1365 (this allows the optics to be interchanged with other disposable sheaths 1365 having different optics by swapping sheaths, in order to easily obtain the desired optical pattern uniquely suited for nerve, brain or other tissue stimulation and avoiding possible contamination from the lens if the lens were left in place). Such a sheath 1365 is pulled over the length of handpiece housing 1332 until the light-transmitting optics or lens 1315 of the tip 1367 is flush or engaged with the opening 1324 of housing 1332. In some embodiments, optics 1315 are configured to project a light pattern 1298 that can be focussed or otherwise manually adjusted to a user-specified width and user-specified length. In some embodiments, the pieces of handpiece 1341 are all manufactured from substantially non-metallic, non-magnetic materials such as plastics, polymers, ceramics or the like. In some embodiments, the light pattern desired from the optics is empirically determined by testing various patterns on various tissues and observing the reaction obtained. In other embodiments, the sheath 1365 provides a clear window that covers tip 1367 and its lens 1315. In some embodiments, tip 1367 and its lens 1315 are configured to be an interchangeable optics mechanism (e.g., a threaded or snap-in imaging adaptor configured to be easily swapped), several different ones of which are provided as a kit with handpiece 1341.

FIG. 13B is a block diagram of a battery-operated tissue-conditioning laser handpiece system 1302. In some embodiments, emission of IR and/or visible light from light source 1351 is controlled via a foot-trigger 1308B on controller 1364B being pressed by the foot of user 89, which is operatively coupled to switch 1312B via mechanical linkage that is within cable sheath 1309 (e.g., a flexible plastic rod in a flexible plastic tube). In some embodiments, handpiece 1342 from tip 1367 to cable sheath 1309 are together inserted into disposable sheath 1365 though opening 1313, which is then closed and sealed to cable sheath 1309 (e.g., with a twist-tie or pressure-sensitive adhesive). In some embodiments, other options for sheath 1365 are as described in the other figures herein. In some embodiments, a scanner 1362 (e.g., a two-directional galvoscanner, as are well known in the art) is operably connected to controllably scan the laser output beam in a specified pattern 1390 (e.g., a raster or other suitable scan pattern having a defined and/or user-settable area or lateral extent). In some embodiments, a temperature sensor 1353 receives temperature data from each area being irradiated (as the laser beam goes out through the scanner, IR energy for temperature measurements comes the opposite way through the scanner to sensor 1353, such that the temperatures being measured automatically correspond to the area to which the laser beam is output) and the controller automatically limits the laser output power once the desired temperature is achieved, but then later reapplies laser energy if the temperature later drops below the desired level. This feedback technique helps maintain the tissue at the desired temperature. In some embodiments, system 1302 includes a timer that automatically terminates the conditioning output energy (and/or provides an audible, visual or other indication to the user to alert them to manually terminate the procedure) once a predetermined desired treatment duration has elapsed. In some embodiments, the output beam uses infrared (IR) laser radiation for the heating function (e.g., wavelengths between 1840 nm and 1950 nm). In some embodiments, this wavelength is tunable (e.g., by controlling the temperature of a laser-diode grating). In some embodiments, a visible-light laser beam is superimposed on the IR laser beam to indicate where the IR energy is being directed. In some embodiments, two different visible wavelengths are used-one to indicate where the IR beam will go when it is activated (e.g., this can be used by the user to adjust the length and width of the scan pattern) and a different wavelength is output simultaneously (or substantially simultaneously) with the IR beam to indicate that the IR beam is on or “hot.” Thus, in some embodiments, up to three or more laser beams are output through the scanner (one for setting the boundaries of the scan pattern, one to indicate the conditioning beam is on, and the conditioning beam itself) and a temperature-measuring IR reading is received the opposite direction, where the sensed signal is used to measure and maintain the temperature by controlling the heating output beam. Other aspects of system 1302 are similar to those described for FIG. 13A.

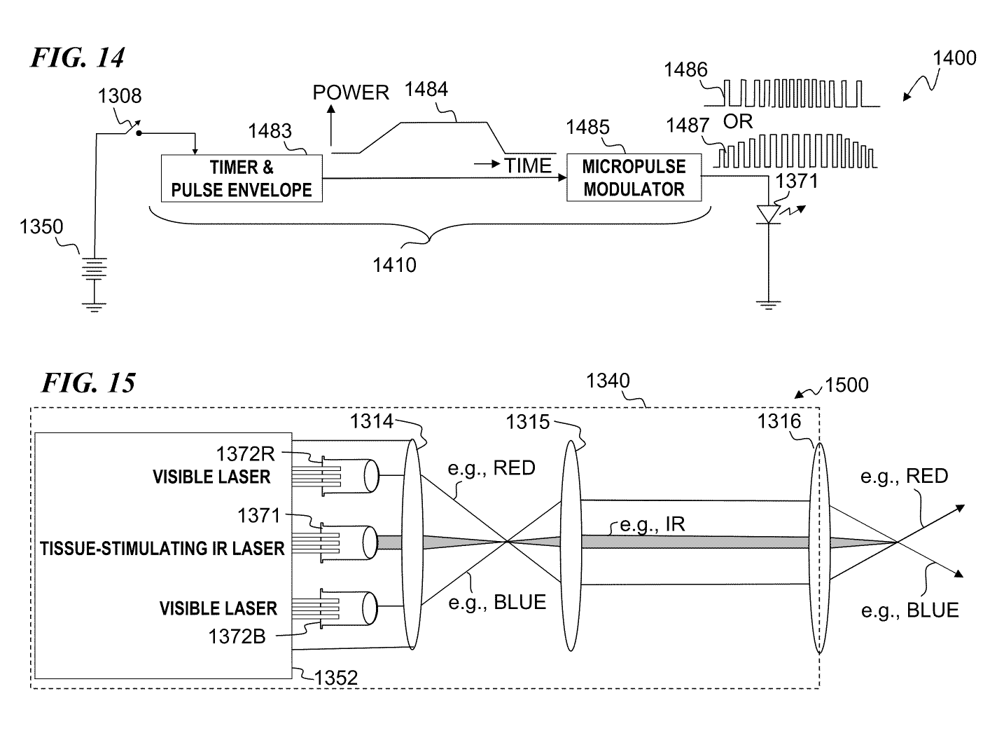

FIG. 13C is a block diagram of a battery-operated tissue-conditioning laser handpiece system 1303. In some embodiments, system 1303 includes a handpiece 1343 having a programming and/or recharging port 1360 (such as a USB port) that is removeably connectable to a computer system 1374 for recharging power system 1350 (e.g., its batteries) and/or for programming and/or transferring data to and/or from the controller of light source 1351. Such programming includes, in some embodiments, one or more predetermined characteristics of the light output such as the duration (e.g., the number of milliseconds each pulse is active), timing (e.g., the number or repetition rate of pulses in one train, or the timing of pulses relative to the scanning position of the scanner which thus allows different amounts or rates of power delivered to different areas of the scan pattern), power (e.g., the total number of watts or the watts per area), shape (e.g., rising trapezoid, flat-top, or falling trapezoid or other shape of each pulse, if desired), timing or like characteristics of the single pulse or pulse train that is initiated by user activation of trigger 1308A (which, in some embodiments, includes one or more internal separately activatable switches). In other embodiments, the programming and/or rechargeability functions described here are combined into any of the other handpieces 1341-1344 or systems of the present invention described herein. In some embodiments, a computer-readable medium stores programs and/or computer-executed instructions that are loaded into personal computer (PC) 1374 and/or into handpiece 1343 that control the method performed in handpiece 1343 and/or the user interface to handpiece 1343. In some embodiments, computer system 1374 includes a user-input device such as keyboard 1375 that communicates with the computer-processing unit of computer system 1374 through connecting link 1376, which may be a hardware-connecting link 1376 or a wireless connecting link 1376. In some embodiments, device 1351 includes two or more of the subsystems (one or more visible-light laser output beams and/or a temperature sensor that measures a temperature of the patient's skin and controls the IR laser output to maintain the desired temperature for the desired time) described for device 1351 in the description of FIG. 13B. In FIGS. 13C and 13D, reference number 1361 represents the control signal source that conveys the time-temperature profile (e.g., time-versus-temperature profile 1484 of FIG. 14) that the respective devices will follow.

FIG. 13D is a block diagram of battery-operated diode-laser-pumped rare-earth-doped fiber emitter tissue-conditioning handpiece system 1304. In some embodiments, the invention uses fibers and pump-diode lasers such as described in U.S. patent application Ser. No. 11/426,302, filed Jun. 23, 2006 and titled “APPARATUS AND METHOD FOR A HIGH-GAIN DOUBLE-CLAD AMPLIFIER,” U.S. patent application Ser. No. 11/488,910, filed Jul. 17, 2006 and titled “APPARATUS AND METHOD FOR GENERATING CONTROLLED-LINEWIDTH LASER-SEED-SIGNALS FOR HIGH-POWERED FIBER-LASER AMPLIFIER SYSTEMS,” U.S. Provisional Patent Application Ser. No. 60/748,379, filed Dec. 7, 2005 and titled “APPARATUS AND METHOD FOR AN ERBIUM-DOPED FIBER FOR HIGH-PEAK-POWER APPLICATION,” and U.S. Provisional Application Ser. No. 60/733,977, filed Nov. 3, 2005 and titled “APPARATUS AND METHOD FOR A WAVEGUIDE WITH AN INDEX PROFILE MANIFESTING A CENTRAL DIP FOR BETTER ENERGY EXTRACTION,” each of which is incorporated herein by reference. In some embodiments, a pump laser diode emits pump light at about 960-micron wavelength, the doping species of the fiber is chosen (using a table of such elements that are well known to persons of skill in the art) to obtain a wavelength suitable for nerve or other tissue stimulation. In some embodiments, controller and laser device 1358 includes electronics and light emitters. In some embodiments, one or more of the light emitters operate with visible wavelengths for pointer use, and one or more light emitters in wavelengths suitable for pumping the fiber emitters 1359 (i.e., the pump lasers emit a wavelength suitable for pumping the fiber laser segment(s) 1359). In some embodiments, fiber emitters 1359 include feedback devices such as mirrors, gratings or the like, and operate as lasers. In other embodiments, the fibers serve as superluminescent emitters, wherein spontaneous emission of the fibers is amplified in the fibers. Other aspects of system 1304 are as described in the above figure descriptions. In some embodiments, reference number 1361 represents the control signal source that conveys the time-temperature profile (e.g., time-versus-temperature profile 1484 of FIG. 14) that the respective devices will follow, wherein the profile is optionally selected by manually activated triggers 1327 and 1317.

FIG. 13E is a block diagram of a combined light source or laser assembly 1305. In some embodiments, assembly 1305 includes an IR-laser-diode emitter 1371 that emits a wavelength of light useful for tissue conditioning (heating) and a front lens 1354. Some embodiments further include a visible laser or LED 1372 and a beam-combiner optic or optics 1353 (for example, a highly reflective mirror 1319 and a beam combiner plate 1318, such as a dichroic mirror that is highly reflective of one wavelength (e.g., the visible-light wavelength of emitter 1372) and highly transmissive of another wavelength (e.g., the stimulation-light wavelength of emitter 1371). Some embodiments further include a surgical (e.g., debriding, for “cleaning up” rough edges of a pre-existing wound) beam generated by a high-power laser 1373 and combined using a beam-combiner optic or optics 1316 (for example, a highly reflective mirror 1317 and a beam combiner plate 1318, such as a dichroic mirror that is highly reflective of one wavelength (e.g., the visible-light wavelength of emitter 1373) and highly transmissive of another wavelength (e.g., the stimulation-light wavelength of emitter 1371) similar to beam combiner 1353, except for the wavelengths for which the dichroic mirror is configured). In some embodiments, combining the two or more beams into a single beam makes the downstream optics (such as scanner 1362 and output optics 1354) simpler. In other embodiments such as described below for FIG. 15, light source 1371 generates two or more beams (e.g., parallel beams, in some embodiments), wherein the handpiece is configured to deliver the light in the separate beams to the desired location using suitable optics (e.g., some embodiments include two or more visible pointer beams that form separate beams that form separated spots, lines or patterns when the device is not at the proper distance from the patient, and the optics is arranged to focus the two visible beams into a single spot, line or pattern only when the invisible (IR) stimulation beam is at the proper distance and/or focus to deliver the desired power density).

In some embodiments, one or more visible-light sources 1372 emit visible indicator light (i.e., light having one or more visible wavelengths suitable for indicating to a user where the conditioning (heating) light or surgical (e.g., debriding) light will be delivered), which is coupled by light-beam combiner and/or coupler 1353 to combine with the optical beam from conditioning-wavelength laser 1371. In some embodiments, visible-light sources 1372 include one or more visible-light LEDs, incandescent lamps, and/or laser diodes emitting light at one or more different wavelengths (e.g., 0.45-micron blue light (e.g., gallium-indium nitride devices), 0.55-micron green light (e.g., gallium-indium nitride LED or laser-diode devices), 0.63-micron red light (e.g., gallium-arsenide LED or laser-diode devices), or other wavelengths useful for pointing and/or delivering to the user function-state information, such as different colors or pulsing characteristics to indicate which function has been selected) under control of light-emitting-source controller 1352.

In some embodiments, one or more high-power laser sources 1373 emit high-power laser light (or very-short-pulse laser light), which is coupled by light-beam combiner 1316 and/or coupler 1353 into the output beam. In some embodiments, high-power laser sources 1373 include one or more high-power lasers or laser diodes or optically-pumped-fiber lasers emitting light at one or more different wavelengths (e.g., 1.55 microns, or other wavelengths useful for surgical purposes) under control of light-emitting-source controller 1352. In some embodiments, the high-power laser light effects an ablating, burning or cutting operation where heat results from the laser interaction with the tissue (i.e., absorbing photon energy from the laser light and converting it to heat). This can be used for laser debridement of wounds during or before laser conditioning with the same beam or a different beam of light. This can result in cauterizing the surrounding tissue and reducing bleeding. In other embodiments, the continuous wave laser, like the carbon-dioxide laser operating around 10.6 microns, can be used for tissue removal or wound debridement. In other embodiments, the pulse laser light (e.g., from one or more pulsed lasers that concentrate power into a very short time period, such as are described in U.S. Patent Application Publication US 2004 0243111 A1 by Mark Bendett et al. and U.S. Patent Application Publication US 2004 0243112 A1 by Mark Bendett et al., both of which are incorporated by reference) effects a very fast ablation or tiny explosion that removes tissue with substantially no heating of surrounding or underlying tissue.

FIG. 13F is a block functional diagram of battery-operated tissue-conditioning laser handpiece system 1305 such as shown in FIG. 13E described above. Box 1308F shows various input indications received from a user-manipulating trigger 1308A of FIG. 13A or foot-activated switch 1308B of FIG. 13B described above. For example, in some embodiments, an array of one or more buttons and/or a rotateable thumbwheel are activated by the user to initiate one or more functions (e.g., turning on the pointer laser, the nerve-activation laser, or the therapeutic laser, or changing their function). In some embodiments, a single click on a button will cause one function to be performed, while two clicks in short succession produce a different function. In some embodiments, the USB interface 1360 allows the program in programmable controller 1377 to be changed, and/or provides a charging mechanism for batteries 1350, which are later used to power programmable controller 1377 for functionality and power controller 1359 that is used to drive the laser and/or LED light source(s) (e.g., 1371 and 1372). In some embodiments, a display or other function-indicator device 1317 is provided (e.g., and LCD screen or one or more LEDs of one or more colors) that displays text and/or graphics to show the activation state of the laser(s), and their characteristics such as power, temperature, duration so far of the treatment or remaining time of treatment and the like.

FIG. 14 is a block circuit diagram of battery-operated tissue-conditioning laser handpiece system 1400. In some embodiments, power source 1350 is operatively connected to circuit 1410, which provides drive current to stimulation-wavelength laser 1371. In some embodiments, circuit 1410 includes a timer and pulse-envelope (e.g., a one-shot) circuit 1483 that outputs macro pulse envelope waveform 1484 of a suitable shape (e.g., gradually rising leading edge to control rate of temperature rise, steady middle to maintain temperature, and gradually falling trailing edge or other shape), repetition and duration, which signal is then optionally modulated with micro-pulse modulator 1485 to obtain a suitable train of one or more shorter-duration pulses, such as waveform 1486 (pulse-rate and/or pulse-width modulation) or 1487 (amplitude modulation), and laser diode 1371 outputs conditioning light having a corresponding amplitude light output (some embodiments switch the order of components, placing the micro-pulse circuit 1483 first and use the envelope circuit 1483 as an envelope modulator, other embodiments use other orders of components or substitute circuit functions (e.g., software or microcode control of a power transistor driven by a microcontroller)). In other embodiments, circuit 1410 is simply a direct drive or variable-intensity direct drive of an infrared laser diode 1371 from switch or trigger-based intensity controller 1308.

FIG. 15 is a block diagram of focus-indicating tissue-conditioning laser handpiece system 1500. In some embodiments, one, two, or more visible laser diodes 1372 are arranged around (e.g., in some embodiments, one on either side and parallel with) the beam of the tissue-conditioning laser diode 1371. A single lens 1315 or a series of two or more lenses (e.g., 1314, 1315, and 1316) are used to collimate and focus the tissue-conditioning beam to a point, or a suitable shape of a desired size. This optical path causes the two pointer beams (e.g., a red pointer beam from the top laser 1372R and a blue beam from the bottom laser 1372B) to cross at the optimal focus depth to obtain the desired focus or treatment distance of the non-visible IR tissue-conditioning beam. If the lens 1316 at the tip of the handpiece 1340 is too close, the blue-beam's spot will be below the red-beam's spot, or if the lens 1316 at the tip of the handpiece 1340 is too far from the nerve, the blue-beam's spot will be above the red-beam's spot. When at the correct focus, the red spot and blue spot will coincide—be on top of one other. In some embodiments, the projection light of the two colors provides lines or other patterns rather than spots.

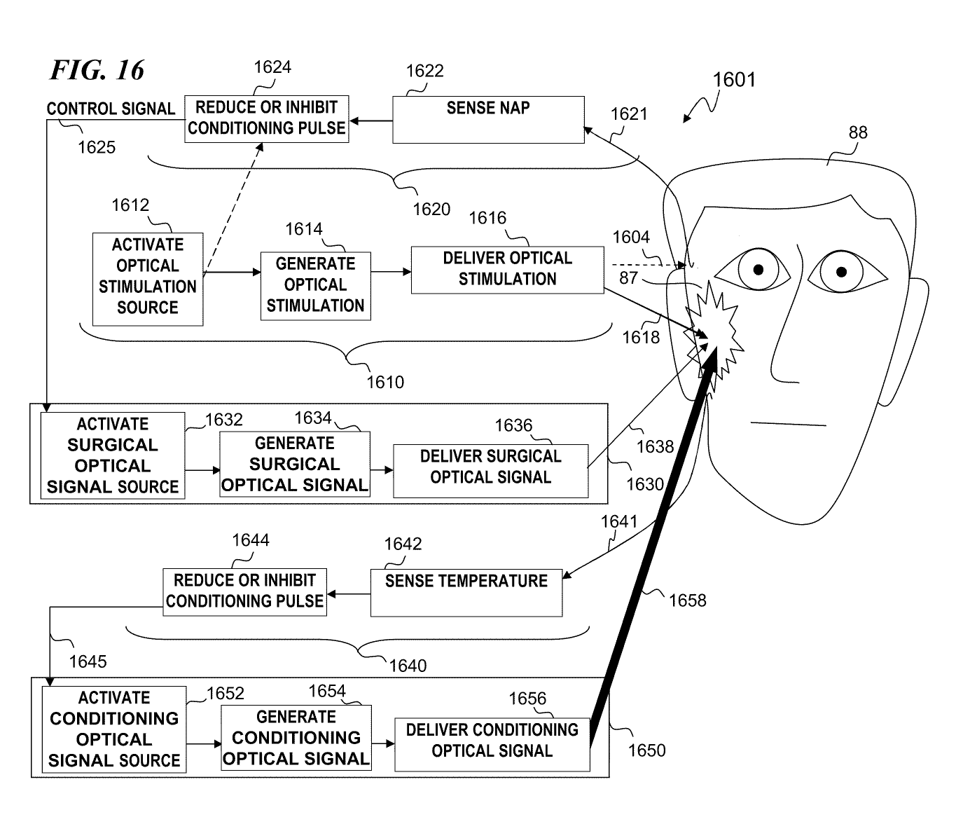

FIG. 16 is a block diagram of surgery-inhibiting wound debriding tissue-conditioning system 1601. In some embodiments of the present invention the tissue-conditioning instrument further includes a tissue-ablating laser beam that is used on an existing wound (such as a battlefield injury) to debride and/or otherwise clean up rough edges and/or remove tissue that is dead or that will die from the wound trauma. In some embodiments, while the debriding operation is being performed, a tissue-conditioning beam heats the surrounding tissue to induce wound healing processes (such as inducing Hsp70 and/or other heat-shock proteins). In some embodiments, system 1601 includes some or all of the functions described for system 400A in FIG. 4A of U.S. patent application Ser. No. 11/536,642 except that, in addition, system 1601 of the present invention also provides the selectively activatable function that conditions a tissue of patient 88 using an optical signal 1658 focussed and/or scanned across a suitable tissue area and to a suitable tissue depth (to a predetermined temperature and conditioning time), while or as an auxiliary function of a wound-debridement preparation to a later wound-treatment main surgery or other surgical-operation. For those situations where debridement may take the ten-or-so minutes that the conditioning protocol calls for, the conditioning does not add any time for the debridement and other surgical functions that will take place. That is, in some embodiments, the laser debridement to clean up the edges of a wound that will later be sutured and otherwise treated will, at the same time, project a less-focussed and/or scanned beam of tissue conditioning light in a pattern to heat the surrounding tissue to generate heat-shock proteins for better wound healing. In some embodiments, the wound-debridement function is temporarily inhibited whenever a nerve or intact tissues (which are intended to be kept intact) is detected within the debridement beam. For example, some embodiments project a nerve-stimulating optical signal to the location that debridement will occur, so that only if no nerve stimulation is detected will the debridement beam be allowed to ablate the rough edges of the wound, while if nerve stimulation is detected, then the debridement beam be inhibited.

In some embodiments, additional functions are added to the devices of the present invention. As described in U.S. patent application Ser. No. 11/536,642 (incorporated herein by reference), in some embodiments, if a sensory nerve 87 (and/or a motor nerve) is stimulated by the optical stimulation signal 1618 (e.g., a laser signal pulse having an IR wavelength of about 1.8 microns, in some embodiments) sufficiently to trigger an action potential (e.g., a compound nerve-action potential, or CNAP), that nerve stimulation is sensed (e.g., by the nerve's electrical signal sensed by a needle-sized hook probe or other suitable probe along the stimulated nerve a short distance away (e.g., towards the brain if the nerve is a sensory nerve, and/or towards the muscle if the nerve is a motor nerve), or by a mechanical sensor such as a small piezo sensor or strain gauge that outputs an electrical signal if the muscle twitches due to the nerve being stimulated) and if sense signal 1621 indicates the nerve was stimulated, then surgical-debridement or therapeutic-operation laser signal 1638 is suppressed (inhibited or sufficiently reduced) to avoid damaging the stimulated nerve 87. In some embodiments, the nerve-stimulation signal 1618 is used for stimulating a nerve, and the detected nerve signal 1621 or its results are sensed and used to inhibit the debridement or surgical laser beam 1638.

Referring again to FIG. 16, in some embodiments, system 1601 includes a stimulation unit 1610 that outputs an optical signal 1618 that is at least partially effective at stimulating a nerve 87 of patient 88. In some embodiments, stimulation unit 1610 includes block 1612 (e.g., a trigger such as 1308A of FIG. 13A described above) that activates an optical stimulation source, block 1614 (e.g., an IR laser diode such as device 1351 of FIG. 13A described above) that generates an optical stimulation signal, and unit 1616 (such as an optical fiber and/or lens system that directs and/or focuses optical signal 1618 onto a particular location with desired light-beam characteristics (such as size, power, shape, and the like) to stimulate nerve 87). In some embodiments, a suitable probe (such as a needle hook adapted to attach to an empirically determined location on nerve 87 to detect whether an action potential has been triggered) generates a relatively small signal that is amplified and/or conditioned by block 1622 (e.g., a sensitive low-current differential operational amplifier circuit) and block 1624 (e.g., an analog and/or digital logic circuit that examines the output signal from block 1622 in relationship to the stimulation trigger from block 1612 to determine whether to reduce or inhibit the surgical signal and/or by how much to reduce the cutting signal) that generates control signal 1625. In some embodiments, control signal 1625 controls one or more aspects of block 1630, which is what generates and/or controls the surgical optical pulses 1638. In some embodiments, stimulation optical signals 1618 and/or surgical optical signals 1638 also include a visible pointer signal to show the user where the stimulation and/or surgery is taking or is soon to be taking place, and optionally the stimulation signal, the visible pointer, and the cutting optical signal are all generated from a single unit (e.g., an optical unit such as unit 1341 of FIG. 13B) and/or are all combined and delivered through a single output lens and/or optical fiber. In some embodiments, block 1630 includes an activation circuit 1632 that selectively activates the laser (or inhibits its activation based on signal 1625), a laser source 1634 that generates the laser signal used for the surgical procedure, and delivery optics 1636 (e.g., lenses, diffractive gratings, or optical fibers) used to precisely deliver the surgical laser energy 1638 to the location desired. In some embodiments, block 1630 is implemented as a separate laser and controller (e.g., such as a LASIK ophthalmic surgical optical source (Laser-Assisted In Situ Keratomileusis, using an excimer laser)) whose output is controlled and/or inhibited by control signal 1625, and delivered by an optical fiber or combined into a single optical fiber with the stimulation signal 1618 for delivery and placement onto the surgical site on patient 88. In other embodiments, a single hand-held self-powered (e.g., via internal batteries or other power source 1350, as shown in FIG. 13A and the like above) stimulation and surgical laser handpiece is implemented and the cutting operation is controlled and/or inhibited by control signal 1625 generated by an integrated or by a separate sensing and inhibition unit 1620 (e.g., in some embodiments, system 1601 is implemented and contained in a single handpiece such as 1302 of FIG. 13B described above).

In various embodiments, the sensing, inhibition and/or control functions of sensing and inhibition unit 1620 are used in combination with or integrated into any of the nerve-simulation systems described herein or described in U.S. patent application Ser. No. 11/536,642 or U.S. patent application Ser. No. 11/420,729 filed on May 26, 2006 entitled “APPARATUS AND METHOD FOR OPTICAL STIMULATION OF NERVES AND OTHER ANIMAL TISSUE” (which are incorporated herein by reference).

In some embodiments, system 1601 further includes a conditioning unit 1650 and a conditioning-control-feedback unit 1640. In some embodiments, conditioning-control-feedback unit 1640 includes a temperature sensing unit (such as an IR camera) 1642 that senses a temperature 1641 of the area being conditioned, and an inhibition circuit 1644 that generates an inhibit signal 1645 that reduces or inhibits the conditioning signal when the desired temperature is achieved, but re-enables the conditioning signal 1658 if the temperature again falls below the desired temperature. In some embodiments, conditioning unit 1650 includes a block 1652 that activates the conditioning optical signal source 1654 whose light output is delivered to block 1656 e.g., a laser beam scanner or optical fiber system that delivers conditioning signal 1658 to patient 88. The operation of unit 1640 and 1650 can be performed at the same time that the surgical signal 1638 is used to clean up or prepare the wound on patient 88 for a later surgical operation. In other embodiments, surgical optical signal can be the main surgical operation such that the conditioning and the main surgery are performed simultaneously or at least partially overlapped in time.

In some embodiments, a local anesthetic and/or analgesic 1604 (e.g., such as Novocain or acupuncture; see FIG. 16) can be administered “upstream” (e.g., between the surgical site and the brain) along a sensory nerve to prevent pain and discomfort during the operation, while the nerve stimulation and sensing of the present invention is still functional to locate and preserve the nerve at the site of the operation.

In some embodiments, the surgical area is defined by a mask (e.g., surgical adhesive tape) or marked boundary that is optically sensed and outside of which the cutting and/or conditioning function is inhibited. For example, in some embodiments, the area to be treated is delineated by a marked line or shading (e.g., ink or a fluorescent dye) that indicates where cutting is permitted, and only when the visible pointer beam is projected on the allowed area is the cutting beam activated, but when the pointer is outside the allowed area, the cutting is inhibited. This additional inhibition function provides an additional safeguard as to where cutting is performed.

TABLE OF REFERENCES (EACH OF WHICH IS INCORPORATED HEREIN BY REFERENCE;
THE REFERENCES ARE REFERRED TO ABOVE AS (LASTNAME YEAR)):

Akita Mouse Datasheet, in Online <http://jaxmice.jax.org/strain/006580.html>, P. information,
Editor, Jackson Labs
Baskaran, H., et al. (2001), Poloxamer-188 improves capillary blood flow and tissue viability in a
cutaneous burn wound. J. Surg. Res. 101: 56-61.
Beckham, J. T., et al. (2004), Assessment of cellular response to thermal laser injury through
bioluminescence imaging of heat shock protein 70. Photochem. Photobiol. 79: 76-85.
Beckham, J. T., et al. (2007), 2007 ASLMS abstracts - disclosure and FDA status. Lasers in Surgery
and Medicine 39: 87-100.
Beckham, J. T., et al. (2004), Assessment of cellular response to thermal laser injury through
bioluminescence imaging of heat shock protein 70. Photochem. Photobiol., 2004. 79(1): p. 76-85.
Beere, H. M., et al. (2000), Heat-shock protein 70 inhibits apoptosis by preventing recruitment of
procaspase-9 to the Apaf-1 apoptosome. Nat. Cell Biol. 2: 469-475.
Benn, S. I., et al. (1996), Particle-mediated gene transfer with transforming growth factor-beta1 cDNAs
enhances wound repair in rat skin. J. Clin. Invest. 98: 2894-2902.
Bowman, P. D., et al. (1997), Survival of human epidermal keratinocytes after short-duration high
temperature: synthesis of HSP70 and IL-8. Am. J. Physiol. 272: C1988-1994.
Braddock, M., et al. (1999), Current therapies for wound healing: electrical stimulation, biological
therapeutics, and the potential for gene therapy. Int. J. Dermatol. 38: 808817.
Brem, H., et al. (2006), Evidence-based protocol for diabetic foot ulcers. Plast. Reconstr. Surg., 2006.
117(7 Suppl): p. 193S-209S; discussion 210S-211S.
Breyer, M. D. (2004), Animal Models of Diabetic Complications Consortium: Update Report, in Online
http://www.amdcc.org/documents/reports/BreyerAR2004.pdf.
Cao, Y., et al. (1999), TGF-beta1 mediates 70-kDa heat shock protein induction due to ultraviolet
irradiation in human skin fibroblasts. Pflugers Arch., 1999. 438(3): p. 239-44.
Capon, A. and S. Mordon (2003), Can thermal lasers promote skin wound healing? Am. J. Clin.
Dermatol. 4: 1-12.
Capon, A., et al. (2001), Laser assisted skin closure (LASC) by using an 815-nm diode-laser system
accelerates and improves wound healing. Lasers Surg. Med., 2001. 28(2): p. 168-75.
Capon, A., et al. (2008), Scar Prevention by Laser-Assisted Scar Healing (LASH): A Pilot Study Using
an 810-nm Diode-Laser System. Lasers Surg. Med. 40: 443-445
Contag, C. H. and M. H. Bachmann (2002), Advances in in vivo bioluminescence imaging of gene
expression. Annu. Rev. Biomed. Eng. 4: 235-260.
Contag, C. H., et al. (1997), Visualizing gene expression in living mammals using a bioluminescent
reporter. Photochem. Photobiol. 66: 523-531.
Contaldo, C., et al. (2007), The influence of local and systemic preconditioning on oxygenation,
metabolism and survival in critically ischaemic skin flaps in pigs. J. Plast. Reconstr. Aesthet. Surg.
Currie, R. W., et al. (1988), Heat-shock response is associated with enhanced postischemic ventricular
recovery. Ciro Res. 63: 543-549.
Danon, D., et al. (1989), Promotion of wound repair in old mice by local injection of macrophages.
Proc. Nati. Aced. Sci. USA 86: 2018-2020.
Davidson, J. M. and S. I. Benn (1996), Biochemical and molecular regulation of angiogenesis and
wound repair. In: Cellular and Molecular Pathogenesis, A. E. Sirica (ed), Raven Press, New York,
1996: p. 79-108.
Davidson, J. M. (1998), Animal models for wound repair. Arch. Dermatol. Res., 1998. 290 Suppl: p. S1-
11.
Desmettre, T., et al. (2001), Heat shock protein hyperexpression on chorioretinal layers after
transpupillary thermotherapy. Invest. Ophthalmol. Vis. Sci. 42: 2976-2980.
Diller, K. R. (2006), Stress protein expression kinetics. Annu. Rev. Biomed. Eng. 8: 403-424.
Dinh, H. K., et al. (2001), Gene expression profiling of the response to thermal injury in human cells.
Physiol Genomics 7: 3-13.
Duncan, R. F. (2005), Inhibition of Hsp90 function delays and impairs recovery from heat shock. Febs.
J., 2005. 272(20): p. 5244-56.
Eldor, R., et al. (2004), New and experimental approaches to treatment of diabetic foot ulcers: a
comprehensive review of emerging treatment strategies. Diabet. Med., 2004. 21(11): p. 1161-73.
Erdos, G, et al. (1995), Heat-induced bFGF gene expression in the absence of heat shock element
correlates with enhanced AP-1 binding activity. J. Cell Physiol. 164: 404-413.
Flanders, K. C., et al. (1993), Hyperthermia induces expression of transforming growth factor-beta s in
rat cardiac cells in vitro and in vivo. J. Clin. Invest. 92: 404410.
Florin, L., et al. (2006), Delayed wound healing and epidermal hyperproliferation in mice lacking JunB
in the skin. J. Invest. Dermatol. 126: 902-911.
Frydman, J. (2001), Folding of newly translated proteins in vivo: the role of molecular chaperones.
Annu. Rev. Biochem., 2001. 70: p. 603-47.
Frykberg, R. G. (1999), Epidemiology of the diabetic foot: ulcerations and amputations. Adv. Wound
Care, 1999. 12(3): p. 139-41.
Gabai, V. L., et al. (1997), Hsp70 prevents activation of stress kinases. A novel pathway of cellular
thermotolerance. J. Biol. Chem. 272: 18033-18037.
Garrido, C., et al. (2001), Heat shock proteins: endogenous modulators of apoptotic cell death.
Biochem. Biophys. Res. Commun. 286: 433-442.
Gowda, A., et al. (1998), Cardioprotection by local heating: improved myocardial salvage after
ischemia and reperfusion. Ann. Thorac Surg. 65: 1241-1247.
Gyurko, R., et al. (2006), Chronic hyperglycemia predisposes to exaggerated inflammatory response
and leukocyte dysfunction in Akita mice. J. Immunol., 2006. 177(10): p. 7250-6.
Hale, G. Q. and M. R. Querry (1973), Optical constants of water in 200 nm to 200 um wavelength region.
Applied Optics 12: 555-563.
Harder, Y., et al. (2004), Improved skin flap survival after local heat preconditioning in pigs. J. Surg.
Res. 119: 100-105.
Hargitai, J., et al. (2003), a heat shock protein co-inducer, acts by the prolonged activation of heat
shock factor-1. Biochem. Biophys. Res. Commun, 2003. 307(3): p. 689-695.
Harris, M. I., et al. (1998), Prevalence of diabetes, impaired fasting glucose, and impaired glucose
tolerance in U.S. adults. The Third National Health and Nutrition Examination Survey, 1988-1994.
Diabetes Care, 1998. 21(4): p. 518-24.
Heppleston, A. G. and J. A. Styles (1967), Activity of a macrophage factor in collagen formation by
silica. Nature 214: 521-522.
Hoyte, L. C., et al. (2006), Improved regional cerebral blood flow is important for the protection seen in
a mouse model of late phase ischemic preconditioning. Brain Res, 2006. 1121(1): p. 231-7.
Ishibashi, T., et al. (1994), A novel dual specificity phosphatase induced by serum stimulation and heat
shock. J. Biol. Chem. 269: 29897-29902.
Jaattela, M. and D. Wissing (1992), Emerging role of heat shock proteins in biology and medicine.
Ann. Med. 24: 249-258.
Jaattela, M., et al. (1992), Major heat shock protein hsp70 protects tumor cells from tumor necrosis
factor cytotoxicity. Embo J, 1992. 11(10): p. 3507-12.
Kabakov, A. E., et al. (2002), Stressful preconditioning and HSP70 overexpression attenuate
proteotoxicity of cellular ATP depletion. Am. J. Physiol. Cell Physiol. 283: C521-534.
Kabakov, A. E. and V. L. Gabai (1997), Heat shock proteins and cytoprotection: ATP-deprived
mammalian cells. 1997, New York: Springer. 237 p.
Kanamori, S., et al. (1999), Induction of vascular endothelial growth factor (VEGF) by hyperthermia
and/or an angiogenesis inhibitor. Int. J. Hyperthermia. 15: 267-278.
Keyse, S. M. and E. A. Emslie (1992), Oxidative stress and heat shock induce a human gene encoding a
protein-tyrosine phosphatase. Nature 359: 644-647.
Kiang, J. G., et al. (2004), Geldanamycin treatment inhibits hemorrhage-induced increases in KLF6 and
iNOS expression in unresuscitated mouse organs: role of inducible HSP70. J. Appl. Physiol. 97: 564-
569.
Kim, J. M., et al. (2004), Effect of thermal preconditioning before excimer laser photoablation. J.
Korean Med. Scit. 19: 437-446.
Kim, D., et al. (1995), Heat shock protein hsp70 accelerates the recovery of heat-shocked mammalian
cells through its modulation of heat shock transcription factor HSF1. Proc Natl Acad Sci USA, 1995.
92(6): p. 2126-30.
Laplante, A. F., et al. (1998), Expression of heat shock proteins in mouse skin during wound healing. J.
Histochem. Cytochem., 1998. 46(11): p. 1291-301.
Leger, J. P., et al. (2000), Confocal microscopic localization of constitutive and heat shock-induced
proteins HSP70 and HSP27 in the rat heart. Circulation 102: 1703-1709.
Lepore, D. A., et al. (2001), Role of priming stresses and Hsp70 in protection from ischemia-reperfusion
injury in cardiac and skeletal muscle. Cell Stress Chaperones 6: 93-96.
Li, Y., et al. (2003), Retinal preconditioning and the induction of heat-shock protein 27. Invest.
Ophthalmol. Vis. Sci. 44: 1299-1304.
Lipsky, B. A., et al. (2004), Diagnosis and treatment of diabetic foot infections. Clin Infect Dis, 2004.
39(7): p. 885-910.
Lipsky, B. A., et al. (2006), Diagnosis and treatment of diabetic foot infections. Plast Reconstr Surg,
2006. 117(7 Suppl): p. 212S-238S.
Madio, D. P., et al. (1998), On the feasibility of MRI-guided focused ultrasound for local induction of
gene expression. J Magn Reson Imaging 8: 101-104.
Marber, M. S., et al. (1995), Overexpression of the rat inducible 70-kD heat stress protein in a
transgenic mouse increases with the resistance of the heart to ischemic injury. J Clin Invest, 1995.
95(4): p. 1446-1456.
Martin, P. (1997), Wound healing--aiming for perfect skin regeneration. Science, 1997. 276(5309): p.
75-81.
Mayer, M. P. and B. Bukau (2005), Hsp70 chaperones: cellular functions and molecular mechanism.
Cell Mol Life Sci, 2005. 62(6): p. 670-84.
McMurtry, A. L., et al. (1999), Expression of HSP70 in healing wounds of diabetic and nondiabetic
mice. J Surg Res 86: 36-41.
Mehlen, P., et al. (1996), Human hsp27, Drosophila hsp27 and human alphaB-crystallin expression-
mediated increase in glutathione is essential for the protective activity of these proteins against
TNFalpha-induced cell death. Embo J, 1996. 15(11): p. 2695-706.
Merchant, F. A., et al. (1998), Poloxamer 188 enhances functional recovery of lethally heat-shocked
fibroblasts. J Surg Res 74: 131-140.
Minowada, G. and W. J. Welch (1995), Clinical implications of the stress response. J Clin Invest, 1995.
95(1): p. 3-12.
Morimoto, R. I., et al. (1996), The transcriptional regulation of heat shock genes: a plethora of heat shock
factors and regulatory conditions. Exs 77: 139-163.
Henriquea, F. C. and A. R. Moritz (1947), Studies of thermal injury in the conduction of heat to and
through skin and the temperatures attained therein. Am J Pathol 23: 531-549.
Morris, S. D., et al. (1996), Specific induction of the 70-kD heat stress proteins by the tyrosine kinase
inhibitor herbimycin-A protects rat neonatal cardiomyocytes. A new pharmacological route to stress
protein expression? J Clin Invest, 1996. 97(3): p. 706-12.
Mosser, D. D., et al. (1997), Role of the human heat shock protein hsp70 in protection against stress-
induced apoptosis. Mol. Cell Biol. 17: 5317-5327.
Ng, K. Y., et al. (2004), Effect of heat preconditioning on the uptake and permeability of R123 in brain
microvessel endothelial cells during mild heat treatment. J. Pharm. Sci., 2004. 93(4): p. 896-907.
Nollen, E. A., et al. (1999), In vivo chaperone activity of heat shock protein 70 and thermotolerance.
Mol. Cell Biol, 1999. 19(3): p. 2069-79.
O'Connell-Rodwell, C. E., et al. (2008 Submitted), In vivo analysis of Hsp70 Induction following Pulsed
Laser Irradiation in a Transgenic Reporter Mouse. Journal of Biomedical Optics.
O'Connell-Rodwell, C. E., et al. (2004), A genetic reporter of thermal stress defines physiologic zones
over a defined temperature range. Faseb. J. 18: 264-271.
Oh, H. J., et al. (1997), Hsp110 protects heat-denatured proteins and confers cellular thermoresistance.
J. Biol. Chem., 1997. 272(50): p. 31636-40.
Parsell, D. A. and S. Lindquist (1993), The function of heat-shock proteins in stress tolerance: degradation
and reactivation of damaged proteins. Annu Rev Genet 27: 437-496.
Pespeni, M., et al. (2005), In vivo stress preconditioning. Methods 35: 158-164.
Pockley, A. G. (2002), Heat shock proteins, inflammation, and cardiovascular disease. Circulation
105: 1012-1017.
Ravagnan, L., et al. (2001), Heat-shock protein 70 antagonizes apoptosis-inducing factor. Nat Cell Biol
3: 839-843.
Richard, V., et al. (1996), Delayed protection of the ischemic heart--from pathophysiology to
therapeutic applications. Fundam Clin Pharmacol, 1996. 10(5): p. 409-15.
Ritossa, F. (1962), A new puffing pattern induced by temperature shock and DNP in Drosophila.
Experientia 18: 571-573.
Rylander, M. N., et al. (2005), Correlation of HSP70 expression and cell viability following thermal
stimulation of bovine aortic endothelial cells. J Biomech Eng 127: 751757.
Rylander, M. N., et al. (2006), Optimizing heat shock protein expression induced by prostate cancer laser
therapy through predictive computational models. J Biomed Opt 11: 041113.
Samali A. and T. G. Cotter (1996), Heat shock proteins increase resistance to apoptosis. Exp Cell Res
223: 163-170.
Samali, A. and S. Orrenius (1998), Heat shock proteins: regulators of stress response and apoptosis.
Cell Stress Chaperones, 1998. 3(4): p. 228-36.
Schaffer, M., et al. (2007), Nitric oxide restores impaired healing in normoglycaemic diabetic rats. J.
Wound Care, 2007. 16(7): p. 311-6.
Seppa, L., et al. (2004), Upregulation of the Hsp104 chaperone at physiological temperature during
recovery from thermal insult. Mol. Microbiol. 52: 217-225.
Sherar, M. D., et al. (2001), Interstitial microwave thermal therapy for prostate cancer: method of
treatment and results of a phase I/II trial. J. Urol., 2001. 166(5): p. 1707-14.
Snoeckx, L. H., et al. (2001), Heat shock proteins and cardiovascular pathophysiology. Physiol Rev
81: 1461-1497.
Song, C. W. (1984), Effect of local hyperthermia on blood flow and microenvironment: a review.
Cancer Res. 44: 4721s-4730s.
Souil, E., et al. (2001), Treatment with 815-nm diode laser induces long-lasting expression of 72-kDa
heat shock protein in normal rat skin. Br. J. Dermatol. 144: 260-266.
Thomsen, S. (1991), Pathologic analysis of photothermal and photomechanical effects of laser-tissue
interactions. Photochem Photobiol, 1991. 53(6): p. 825-35.
Topping, A., et al. (2001), Successful reduction in skin damage resulting from exposure to the normal-
mode ruby laser in an animal model. British Journal of Plastic Surgery 54: 144-150.
Torok, Z., et al. (2003), Heat shock protein coinducers with no effect on protein denaturation
specifically modulate the membrane lipid phase. Proc. Natl. Acad. Set. U.S.A., 2003. 100(6): p. 3131-6.
Vigh, L., et al. (1997), Bimoclomol: a nontoxic, hydroxylamine derivative with stress protein-inducing
activity and cytoprotective effects. Nat. Med. 3: 1150-1154.
Vogel, A. and V. Venugopalan (2003), Mechanisms of pulsed laser ablation of biological tissues.
Chem. Rev. 103: 577-644.
Walters, T. J., et al. (1998), HSP70 expression in the CNS in response to exercise and heat stress in rats.
J. Appl. Physiol. 84: 1269-1277.
Wang, M. H., et al. (2004), Forced expression of heat-shock protein 70 increases the secretion of Hsp70
and provides protection against tumour growth. Br. J. Cancer 90: 926-931.
Wilmink, G. J., et al. (2007), Wavelength-dependent dynamics of heat shock protein 70 expression in
free electron laser wounds. In: Thermal Treatment of Tissue: Energy Delivery and Assessment IV
(Ryan T P ed) SPIE: San Jose, CA, USA, 644003-644012.
Wilmink, G. J., et al. (2006), Assessing laser-tissue damage with bioluminescent imaging. J. Biomed. Opt.
11: 041114.
Wilmink, G. J., et al. (TBD), Molecular Imaging-Assisted Optimization of Hsp70 Expression During
Laser Preconditioning for Wound Repair Enhancement. J. Invest. Derm., submitted.
Wirth, D. P., et al. (1996), Wound healing and complementary therapies: a review. J. Altern. Complement
Med. 2: 493-502.
Wu, S. C., et al. (2007), Foot ulcers in the diabetic patient, prevention and treatment. Vasc. Health Risk
Manag., 2007. 3(1): p. 65-76.
Wynn, R. M., et al. (1994), Molecular chaperones: heat-shock proteins, foldases, and matchmakers. J. Lab.
Clin. Med. 124: 31-36.
Yonehara, M., et al. (1996), Heat-induced chaperone activity of HSP90. J. Biol. Chem., 1996. 271(5):
p. 2641-5.
Yoshioka, M., et al. (1997), A novel locus, Mody4, distal to D7Mit189 on chromosome 7 determines
early-onset NIDDM in nonobese C57BL/6 (Akita) mutant mice. Diabetes, 1997. 46(5): p. 887-94.
Young, J. C., et al. (2004), Pathways of chaperone-mediated protein folding in the cytosol. Nat. Rev.
Mol. Cell Biol, 2004. 5(10): p. 781-91.
Young, J. C., et al. (2003), More than folding: localized functions of cytosolic chaperones. Trends
Biochem. Sci., 2003. 28(10): p. 541-7.

In some embodiments, the present invention provides an apparatus that includes a laser device configured to provide a therapeutically effective dose of laser radiation to a first area of tissue of an animal, wherein the dose of laser radiation is controlled to improve a healing response to an injury of the animal. In some embodiments, the animal is a human. In some embodiments, the laser radiation is in the infrared wavelengths. In some embodiments, the laser radiation has a wavelength between about 1800 nm and about 2000 nm. In some such embodiments, the laser radiation has a wavelength between about 1830 nm and about 1950 nm. In some such embodiments, the laser radiation has a wavelength between about 1840 nm and about 1940 nm. In other embodiments, the laser energy for conditioning has a wavelength in the visible and/or ultraviolet range, or in other IR ranges. In some embodiments, the ultimate goal is to achieve a temperature-and-time profile across the suitable depth in the tissue that achieves the desired HSP production and/or otherwise causes enhanced healing of the wound.

In some embodiments, the laser device is a diode laser. In some embodiments, the laser device is a fiber laser (e.g., an optically pumped fiber laser, or an optically pumped fiber amplifier that amplifies a signal generated by a fiber or diode laser seed source) or other suitable type of laser. In some embodiments, the tissue conditioning is preconditioning that is performed before an incision or other surgery. In other embodiments, the tissue conditioning is postconditioning that is performed after an incision or other surgery.

Some embodiments further include a controller that precisely controls a temperature-time history profile to mirror a protocol that has been shown (by empirical testing and/or animal models (such as luc mouse models (for example, a transgenic mouse model generated with a Hsp70-luc-IRES-eGFP construct)) to optimally elicit a desired conditioning effect in a controlled volume. In some embodiments, a plurality of temperature-time history profiles are stored and can be selected by a user based on a particular patient or patient type. In some embodiments, the tissue volume is user-selected depending on the target tissue type and geometry.

In some embodiments, the controller provides a linear or other type of controlled increase/decrease in tissue temperature over time (e.g., the rate of temperature change). Thus, in some embodiments, the temperature does not immediately jump to and stay at the selected high temperature for the entire time, it may ramp up to a selected temperature, and then fluctuate up and down as determined by the testing methods described herein for determining an effective and/or optimal protocol for heating to get the best effect.

In some embodiments, the apparatus further includes a user-controllable laser-beam output configured for debridement of a wound.

Some embodiments of the apparatus further include a scanner mechanism configured to scan a laser beam from the laser device relative to the laser device in a scan pattern across an area of tissue larger than the laser beam. In some embodiments, the scan pattern is a raster scan. Some such embodiments of the apparatus further include a control mechanism that receives user input and based on the user input automatically controls a width and a length of the scan pattern. In some such embodiments, the apparatus further includes a temperature sensor and a timing device operatively coupled to control the laser device such that a predetermined temperature of the first area of tissue is achieved for a predetermined period of time.

Some embodiments further include an endoscopic mechanism operatively coupled to receive laser radiation from the laser device and configured to deliver the laser radiation to a specific location internal to the animal.

Some embodiments of the apparatus further include an imaging device configured to obtain an image of at least a portion of a subject; a scanner mechanism configured to scan a beam of the laser radiation in a scan pattern across an first area of tissue to be conditioned; an imaging-processing device configured to identify a location of at least one fiducial on the subject and to control the scan pattern based at least in part on the identified location of the at least one fiducial; a temperature-sensing device configured to measure a temperature in the first area of tissue; and a controller operatively coupled to the temperature-sensing device, the scanner mechanism, and the laser device and configured to control an amount of laser radiation delivered to the first area based on the measured temperature of the first area. Some such embodiments of the apparatus further include having the scanner mechanism also configured to scan the laser beam in a scan pattern across a second area of tissue to be conditioned; the temperature-sensing device is also configured to measure a temperature in the second area of tissue; and the controller is also configured to control an amount of laser radiation delivered to the second area based on the measured temperature of the second area substantially independent of the amount of laser radiation delivered to the first area.

Some embodiments of the apparatus further include a masking apparatus having an aperture that controls a lateral extent of the dose of laser radiation.

In some embodiments, the apparatus is controlled to raise a temperature of the first area of tissue of the animal to between 41 and 46 degrees C. for between 1 second and 60 minutes. In some embodiments, the apparatus is controlled to raise a temperature of the tissue of the animal to between 43 and 44 degrees C. for between 5 minutes and 20 minutes. In some embodiments, the apparatus is controlled to limit the rate of temperature rise to be no more than a predetermined temperature change per unit time (e.g., to a rate of 5 degrees C. per minute).

In some embodiments, the present invention provides a method that includes providing a source of laser radiation and selectively applying a therapeutically effective dose of the laser radiation to a first area of tissue of an animal, wherein the dose of laser radiation is controlled to improve a healing response to an injury of the animal.

In some embodiments of the method, the selectively applying further comprises scanning a beam of the laser radiation in a scan pattern across an area of tissue larger than the laser beam.

Some embodiments of the method further include receiving user input; and automatically controlling a width and a length of the scan pattern based on the user input.

Some embodiments of the method further include sensing a temperature and controlling the selectively applying based on the measured temperature of the first area such that a predetermined temperature of the first area tissue is achieved for a predetermined period of time.

Some embodiments of the method further include endoscopically delivering the laser radiation to a specific location internal to the animal.

Some embodiments of the method further include obtaining an image of at least a portion of a subject; scanning an output beam of the laser radiation in a scan pattern across an first area of tissue to be conditioned; identifying from the image a location of at least one fiducial on the subject and automatically controlling the scan pattern based at least in part on the identified location of the at least one fiducial; measuring a temperature in the first area of tissue; and controlling an amount of laser radiation delivered to the first area based on the measured temperature of the first area.

Some embodiments of the method further include scanning the laser beam in a scan pattern across a second area of tissue to be conditioned; measuring a temperature in the second area of tissue; and controlling an amount of laser radiation delivered to the second area based on the measured temperature of the second area substantially independent of the amount of laser radiation delivered to the first area.

Some embodiments of the method further include masking a lateral extent of the dose of laser radiation.

Some embodiments of the method further include controlling a temperature of the first area of tissue of the animal to between 41 and 46 degrees C. for between 1 second and 60 minutes. Some embodiments of the method further include controlling a temperature of the first area of the tissue of the animal to between 42 and 45 degrees C. for between 1 minute and 30 minutes. Some embodiments of the method further include controlling a temperature of the first area of the tissue of the animal to between 43 and 44 degrees C. for between 5 minutes and 20 minutes. Some embodiments of the method include controlling a temperature of the first area of the tissue of the animal to a temperature of about 43 degrees C. for about 10 minutes.

In some embodiments, the temperature is controlled to a temperature between 40 and 41 degrees C. In some embodiments, the temperature is controlled to a temperature between 40 and 41 degrees C. In some embodiments, the temperature is controlled to a temperature between 41 and 42 degrees C. In some embodiments, the temperature is controlled to a temperature between 42 and 43 degrees C. In some embodiments, the temperature is controlled to a temperature between 43 and 44 degrees C. In some embodiments, the temperature is controlled to a temperature of about 43 degrees C. In some embodiments, the temperature is controlled to a temperature between 44 and 45 degrees C. In some embodiments, the temperature is controlled to a temperature between 45 and 46 degrees C. In some of each of these embodiments, the temperature is controlled to the stated temperature range for a time duration between 1 second and 120 minutes. In some embodiments, the duration is between 1 second and 1 minute. In some embodiments, the duration is between 1 minute and 5 minutes. In some embodiments, the duration is between 5 minutes and 10 minutes. In some embodiments, the duration is between 10 minutes and 15 minutes. In some embodiments, the duration is between 15 minutes and 20 minutes. In some embodiments, the duration is between 20 minutes and 25 minutes. In some embodiments, the duration is between 25 minutes and 30 minutes. In some embodiments, the duration is between 30 minutes and 35 minutes. In some embodiments, the duration is between 35 minutes and 40 minutes. In some embodiments, the duration is between 40 minutes and 45 minutes. In some embodiments, the duration is between 45 minutes and 50 minutes. In some embodiments, the duration is between 50 minutes and 55 minutes. In some embodiments, the duration is between 55 minutes and 60 minutes. In some embodiments, the duration is between 60 minutes and 65 minutes. In some embodiments, the duration is between 65 minutes and 70 minutes. In some embodiments, the duration is between 70 minutes and 75 minutes. In some embodiments, the duration is between 75 minutes and 80 minutes. In some embodiments, the duration is between 80 minutes and 90 minutes. In some embodiments, the duration is between 90 minutes and 100 minutes. In some embodiments, the duration is between 100 minutes and 120 minutes.

In some embodiments, the present invention provides an apparatus that includes a source of laser radiation; and means for selectively applying a therapeutically effective dose of the laser radiation to a first area of tissue of an animal, wherein the dose of laser radiation is controlled in a manner that increases at least one heat-shock protein to improve a healing response to an injury of the animal.

In some embodiments of this apparatus, the means for selectively applying further comprises means for scanning a beam of the laser radiation in a scan pattern across an area of tissue larger than the laser beam.

Some embodiments of this apparatus further include means for receiving user input; and means for automatically controlling a width and a length of the scan pattern based on the user input.

Some embodiments of this apparatus further include means for sensing a temperature and means for controlling the selectively applying based on the measured temperature of the first area such that a predetermined temperature of the first area tissue is achieved for a predetermined period of time.

Some embodiments of this apparatus further include means for endoscopically delivering the laser radiation to a specific location internal to the animal.

Some embodiments of this apparatus further include means for obtaining an image of at least a portion of a subject; means for scanning an output beam of the laser radiation in a scan pattern across an first area of tissue to be conditioned; means for identifying from the image a location of at least one fiducial on the subject and means for automatically controlling the scan pattern based at least in part on the identified location of the at least one fiducial; means for measuring a temperature in the first area of tissue; and means for controlling an amount of laser radiation delivered to the first area based on the measured temperature of the first area.

Some embodiments of this apparatus further include means for scanning the laser beam in a scan pattern across a second area of tissue to be conditioned; means for measuring a temperature in the second area of tissue; and means for controlling an amount of laser radiation delivered to the second area based on the measured temperature of the second area substantially independent of the amount of laser radiation delivered to the first area.

Some embodiments of this apparatus further include means for masking a lateral extent of the dose of laser radiation.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the invention should be, therefore, determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

What is claimed is:

1. An apparatus comprising:
a laser device configured to provide a therapeutically effective dose of laser radiation to a first area of tissue of an animal, wherein the dose of laser radiation is controlled to improve a healing response to an injury of the animal.

2. The apparatus of claim 1, further comprising a scanner mechanism configured to scan a laser beam from the laser device relative to the laser device in a scan pattern across an area of tissue larger than the laser beam.

3. The apparatus of claim 2, further comprising:
a control mechanism that receives user input and based on the user input automatically controls a width and a length of the scan pattern.

4. The apparatus of claim 2, further comprising:
a temperature sensor and a timing device operatively coupled to control the laser device such that a predetermined temperature of the first area of tissue is achieved for a predetermined period of time.

5. The apparatus of claim 1, further comprising an endoscopic mechanism operatively coupled to receive laser radiation from the laser device and configured to deliver the laser radiation to a specific location internal to the animal.

6. The apparatus of claim 1, further comprising:
an imaging device configured to obtain an image of at least a portion of a subject;
a scanner mechanism configured to scan a beam of the laser radiation in a scan pattern across an first area of tissue to be conditioned;
an imaging-processing device configured to identify a location of at least one fiducial on the subject and to control the scan pattern based at least in part on the identified location of the at least one fiducial;
a temperature-sensing device configured to measure a temperature in the first area of tissue; and
a controller operatively coupled to the temperature-sensing device, the scanner mechanism, and the laser device and configured to control an amount of laser radiation delivered to the first area based on the measured temperature of the first area.

7. The apparatus of claim 6, wherein:
the scanner mechanism is also configured to scan the laser beam in a scan pattern across a second area of tissue to be conditioned;
the temperature-sensing device is also configured to measure a temperature in the second area of tissue; and
the controller is also configured to control an amount of laser radiation delivered to the second area based on the measured temperature of the second area substantially independent of the amount of laser radiation delivered to the first area.

8. The apparatus of claim 1, further comprising a masking apparatus having an aperture that controls a lateral extent of the dose of laser radiation.

9. The apparatus of claim 1, wherein the apparatus is controlled to raise a temperature of the first area of tissue of the animal to between 41 and 46 degrees C. for between 1 second and 60 minutes.

10. The apparatus of claim 1, wherein the apparatus is controlled to raise a temperature of the tissue of the animal to between 43 and 44 degrees C. for between 5 minutes and 20 minutes.

11. A method comprising:
selectively applying a therapeutically effective dose of laser radiation to a first area of tissue of an animal, wherein the dose of laser radiation is controlled to improve a healing response to an injury of the animal.

12. The method of claim 11, wherein the selectively applying further comprises scanning a beam of the laser radiation in a scan pattern across an area of tissue larger than the laser beam.

13. The method of claim 12, further comprising:
receiving user input; and
automatically controlling a width and a length of the scan pattern based on the user input.

14. The method of claim 12, further comprising sensing a temperature and controlling the selectively applying based on the measured temperature of the first area such that a predetermined temperature of the first area tissue is achieved for a predetermined period of time.

15. The method of claim 13, further comprising endoscopically delivering the laser radiation to a specific location internal to the animal.

16. The method of claim 11, further comprising:
obtaining an image of at least a portion of a subject;
scanning an output beam of the laser radiation in a scan pattern across an first area of tissue to be conditioned;
identifying from the image a location of at least one fiducial on the subject and automatically controlling the scan pattern based at least in part on the identified location of the at least one fiducial;
measuring a temperature in the first area of tissue; and
controlling an amount of laser radiation delivered to the first area based on the measured temperature of the first area.

17. The method of claim 16, further comprising:
scanning the laser beam in a scan pattern across a second area of tissue to be conditioned;
measuring a temperature in the second area of tissue; and
controlling an amount of laser radiation delivered to the second area based on the measured temperature of the second area substantially independent of the amount of laser radiation delivered to the first area.

18. The method of claim 11, further comprising masking a lateral extent of the dose of laser radiation.

19. The method of claim 11, further comprising controlling a temperature of the first area of tissue of the animal to between 41 and 46 degrees C. for between 1 minute and 60 minutes.

20. The method of claim 11, further comprising controlling a temperature of the first area of the tissue of the animal to between 43 and 44 degrees C. for between 5 minutes and 20 minutes.

21. An apparatus comprising:
a source of laser radiation; and
means for selectively applying a therapeutically effective dose of the laser radiation to a first area of tissue of an animal, wherein the dose of laser radiation is controlled in a manner that increases at least one heat-shock protein to improve a healing response to an injury of the animal.

22. The apparatus of claim 21, wherein the means for selectively applying further comprises means for scanning a beam of the laser radiation in a scan pattern across an area of tissue larger than the laser beam.

23. The apparatus of claim 22, further comprising:
means for receiving user input; and
means for automatically controlling a width and a length of the scan pattern based on the user input.

24. The apparatus of claim 22, further comprising means for sensing a temperature and means for controlling the selectively applying based on the measured temperature of the first area such that a predetermined temperature of the first area tissue is achieved for a predetermined period of time.

25. The apparatus of claim 23, further comprising means for endoscopically delivering the laser radiation to a specific location internal to the animal.

26. The apparatus of claim 21, further comprising:
means for obtaining an image of at least a portion of a subject;
means for scanning an output beam of the laser radiation in a scan pattern across an first area of tissue to be conditioned;
means for identifying from the image a location of at least one fiducial on the subject and means for automatically controlling the scan pattern based at least in part on the identified location of the at least one fiducial;
means for measuring a temperature in the first area of tissue; and
means for controlling an amount of laser radiation delivered to the first area based on the measured temperature of the first area.

27. The apparatus of claim 26, further comprising:
means for scanning the laser beam in a scan pattern across a second area of tissue to be conditioned;
means for measuring a temperature in the second area of tissue; and
means for controlling an amount of laser radiation delivered to the second area based on the measured temperature of the second area substantially independent of the amount of laser radiation delivered to the first area.

28. The apparatus of claim 21, further comprising means for masking a lateral extent of the dose of laser radiation.

29. The apparatus of claim 21, further comprising means for controlling a temperature of the first area of tissue of the animal to between 41 and 46 degrees C. for between 1 minute and 60 minutes.

30. The apparatus of claim 21, further comprising means for controlling a temperature of the first area of the tissue of the animal to between 43 and 44 degrees C. for between 5 minutes and 20 minutes.
